(19) United States
(12) Patent Application Publication
(10) Pub. No.: US 2011/0191894 A1
(43) Pub. Date: Aug. 4, 2011

(54) METHODS AND COMPOSITIONS RELATING TO CONROLLED INDUCTION OF PLANT SENESCENCE

(75) Inventors: Sarah M. Assmann, State College, PA (US); Cha Young Kim, Gyeong-nam (KR); Jerome Bove, Braine (FR); John Kuk Na, State College, PA (US)

(73) Assignee: The Penn State Research Foundation, University Park, PA (US)

(21) Appl. No.: 12/850,273

(22) Filed: Aug. 4, 2010

Related U.S. Application Data
(60) Provisional application No. 61/231,171, filed on Aug. 4, 2009.

(51) Int. Cl. C12N 15/82; C12N 15/63; A01H 5/00; C12N 5/10

(52) U.S. Cl. 800/278; 435/320.1; 800/298; 800/317.2; 800/314; 800/322; 800/310; 800/305; 800/306; 800/307; 800/309; 800/312; 800/313; 800/315; 800/317.1; 800/317.3; 800/317.4; 800/318; 800/320.1; 800/320; 800/317; 800/320.3; 435/419

(57) ABSTRACT

Methods and compositions for promoting senescence in plants are provided. Methods and compositions for promoting senescence in plants by increased expression of an exogenous or endogenous abscisic-acid-activated protein kinase-interacting protein, AKIP. In specific embodiments, transgenic plants are provided expressing increased abscisic-acid-activated protein kinase-interacting protein, AKIP, during the developmental stage of senescence, thereby promoting enhanced plant senescence.

### REFERENCE TO RELATED APPLICATION

This application claims priority to U.S. Provisional Patent Application Ser. No. 61/231,171, filed Aug. 4, 2009, the entire content of which is incorporated herein by reference.

### GOVERNMENT SPONSORSHIP

This invention was made with government support under Contract No. MCB-03-45251 awarded by the National Science Foundation and Contract No. MOST/KOSEF, R15-2003-012-01001-0 awarded by the Environmental Biotechnology National Core Research Center. The government has certain rights in the invention.

### FIELD OF THE INVENTION

The present invention relates generally to methods and compositions for promoting senescence in plants by increased expression of an exogenous or endogenous abscisic-acid-activated protein kinase-interacting protein, AKIP. In specific embodiments, the present invention relates to transgenic plants expressing a recombinant nucleic acid encoding an AKIP, promoting plant senescence.

### BACKGROUND OF THE INVENTION

Senescence is a developmental stage of plants that is characterized by massive programmed cell death in the affected plant or plant part. For example, leaf senescence is characteristic of many annual plants. During the growth phase of plant development, leaves accumulate nutrients and leaf senescence involves loss of chlorophyll, degradation of proteins, nucleic acids, and lipids and redistribution of nutrients to sink tissues such as developing seeds.

Modulation of senescence has important implications in agriculture. Induced plant senescence is useful in various applications, for instance, to dry and remove soybean, cotton, or potato foliage before harvest of the useful part of the plant, e.g. fruits of soybean and cotton plants, or tubers of potato plants. Currently, chemicals are administered to plants to induce senescence. One example of such a chemical is ethephon, a plant growth regulator which is metabolized by plants to produce the senescence-inducing hormone ethylene. Chemical defoliants may have detrimental environmental and human health effects such that decreased use is desirable.

### BRIEF DESCRIPTION OF THE DRAWINGS

FIG. 1 is a comparison of StAKIP1/2 vs. AKIP1 and AKIP2 using CLUSTAL W (1.83) multiple sequence alignment in which conserved RMM motifs are underlined;

FIG. 2 is a comparison of StAKIP3 vs. AKIP3 using CLUSTAL W (1.83) multiple sequence alignment in which conserved RMM motifs are underlined;

FIG. 3A is a reproduction of a photograph showing the lack of significant effect of transformation with a control expression cassette, pSAG12:GUS, on transgenic Arabidopsis plants;

FIG. 3B is a reproduction of a photograph showing accelerated senescence, as indicated by numerous dead leaves (which appear white in the black and white reproduction of the photograph) indicating that the plants are in the last stage of senescence, in transgenic Arabidopsis plants transformed with a senescence-inducible expression cassette, pSAG12:FLAG-ATAKIP2; and

FIG. 4 shows the results of RT-PCR performed using nucleic acids isolated from three independent transgenic potato lines, demonstrating expression of Flag-AKIP2 due to transformation with pSAG12:Flag-AKIP2, compared to non-transformed wild-type, WT, potato plants, in which this RT-PCR product is not produced.

### SUMMARY OF THE INVENTION

Expression cassettes are provided according to the present invention which includes a nucleic acid encoding an abscisic-acid-activated protein kinase-interacting protein, AKIP, operably linked to a heterologous plant non-constitutive promoter.

Expression cassettes are provided according to the present invention which include a nucleic acid encoding an AKIP operably linked to a senescence-activated gene promoter.

Expression cassettes are provided according to the present invention which include a nucleic acid encoding an AKIP operably linked to a plant cell type-specific promoter.

Expression cassettes are provided according to the present invention which include a nucleic acid encoding an AKIP operably linked to a guard cell-specific promoter.

Expression cassettes are provided according to the present invention which include a nucleic acid encoding an AKIP isolated from a plant selected from the group consisting of: alfalfa, apple, apricot, Arabidopsis thaliana, asparagus, avocado, banana, barley, bean, blackberry, broccoli, cabbage, carrot, cauliflower, celery, cherry, chicory, clover, cotton, cucumber, eggplant, garlic, grape, hemp, lettuce, maize, mango, melon, miscanthus, nectarine, onion, papaya, pea, peach, pear, pepper, pineapple, plum, potato, pumpkin, quince, radish, rapeseed, rice, raspberry, rye, soybean, sorghum, spinach, squash, strawberry, sugarcane, sugarbeet, sunflower, sweet potato, switchgrass, tobacco, tomato, turnip, wheat, zucchini, aster, basil, bay leaf, begonia, chives, chrysanthemum, cilantro, clover, delphinium, dill, eucalyptus, lavender, lemon grass, mint, oregano, parsley, rosemary, savory, sunflower, tarragon, thyme, and zinnia.

Expression cassettes are provided according to the present invention which include a nucleic acid encoding a potato AKIP protein. Expression cassettes are provided according to the present invention which include a nucleic acid encoding a potato AKIP protein having at least 95% identity to SEQ ID No. 2. Expression cassettes are provided according to the present invention which include a nucleic acid encoding a potato AKIP selected from: a protein including the amino acid sequence of SEQ ID No. 2; a protein including the amino acid sequence of SEQ ID No. 4; a protein encoded by the complement of a nucleic acid that hybridizes under highly stringent conditions with the nucleotide sequence of SEQ ID No. 1; a protein encoded by the complement of a nucleic acid that hybridizes under highly stringent conditions with the nucleotide sequence of SEQ ID No. 3; wherein the highly stringent conditions are: hybridization in a solution containing 6×SSC, 5× Denhardt's solution, 30% formamide, and 100 micrograms/ml denatured salmon sperm DNA at 37° C. overnight followed by washing in a solution of 0.1×SSC and 0.1% SDS at 60° C. for 15 minutes; a protein comprising an amino acid sequence that is at least 95% identical to SEQ ID No. 2; and a protein comprising an amino acid sequence that is at least 95% identical to SEQ ID No. 4.

Expression cassettes are provided according to the present invention which include a nucleic acid encoding an AKIP protein including the amino acid sequence of SEQ ID No. 6; a protein including the amino acid sequence of SEQ ID No. 8; a protein including the amino acid sequence of SEQ ID No. 10; a protein encoded by the complement of a nucleic acid that hybridizes under highly stringent conditions with the nucleotide sequence of SEQ ID No. 5; a protein encoded by the complement of a nucleic acid that hybridizes under highly stringent conditions with the nucleotide sequence of SEQ ID No. 7; a protein encoded by the complement of a nucleic acid that hybridizes under highly stringent conditions with the nucleotide sequence of SEQ ID No. 9; wherein the highly stringent conditions are: hybridization in a solution containing 6×SSC, 5× Denhardt's solution, 30% formamide, and 100 micrograms/ml denatured salmon sperm DNA at 37° C. overnight followed by washing in a solution of 0.1×SSC and 0.1% SDS at 60° C. for 15 minutes; a protein including an amino acid sequence that is at least 95% identical to SEQ ID No. 6; a protein including an amino acid sequence that is at least 95% identical to SEQ ID No. 8; and a protein including an amino acid sequence that is at least 95% identical to SEQ ID No. 10.

Expression cassettes are provided according to the present invention which include a nucleic acid encoding an AKIP protein selected from: an AKIP protein including the amino acid sequence of SEQ ID No. 24 and the amino acid sequence of SEQ ID No. 29; an AKIP protein including the amino acid sequence of SEQ ID No. 25 and the amino acid sequence of SEQ ID No. 30; an AKIP protein including an amino acid sequence that is at least 95% identical to the amino acid sequence of SEQ ID No. 24 and including an amino acid sequence that is at least 95% identical to the amino acid sequence of SEQ ID No. 29; and an AKIP protein including an amino acid sequence that is at least 95% identical to the amino acid sequence of SEQ ID No. 25 and including an amino acid sequence that is at least 95% identical to the amino acid sequence of SEQ ID No. 30.

Expression cassettes are provided according to the present invention which include a nucleic acid encoding an AKIP protein selected from: an AKIP protein comprising the amino acid sequence of SEQ ID No. 21 and the amino acid sequence of SEQ ID No. 26; an AKIP protein comprising the amino acid sequence of SEQ ID No. 22 and the amino acid sequence of SEQ ID No. 27; an AKIP protein comprising the amino acid sequence of SEQ ID No. 23 and the amino acid sequence of SEQ ID No. 28; an AKIP protein comprising an amino acid sequence that is at least 95% identical to the amino acid sequence of SEQ ID No. 21 and comprising an amino acid sequence that is at least 95% identical to the amino acid sequence of SEQ ID No. 26; an AKIP protein comprising an amino acid sequence that is at least 95% identical to the amino acid sequence of SEQ ID No. 22 and comprising an amino acid sequence that is at least 95% identical to the amino acid sequence of SEQ ID No. 27; and an AKIP protein comprising an amino acid sequence that is at least 95% identical to the amino acid sequence of SEQ ID No. 23 and comprising an amino acid sequence that is at least 95% identical to the amino acid sequence of SEQ ID No. 28.

Expression cassettes are provided according to the present invention which include a senescence-activated gene promoter which is a 5′ non-coding region of a gene selected from the group consisting of: SPG31; SAG2 (At5g60360); SAG12 (At5g45890), SAG13 (At2g29350); SAG14 (At5g20230); SAG15 (At5g51070); SAG101 (At5g14930); SIRK (At2g19190); WRKY6 (At1g62300); WRKY53 (At4g23810); and WRKY70 (At3g56400).

Expression cassettes are provided according to the present invention which include a senescence-activated gene promoter selected from the group consisting of: SEQ ID No. 11, SEQ ID No. 12 and SEQ ID No. 19.

Expression cassettes are provided according to the present invention which include a cell-type specific promoter including the nucleic acid sequence SEQ ID No. 13.

Expression vectors including an expression cassette encoding an AKIP operably linked to a heterologous plant non-constitutive promoter are provided according to the present invention.

Transgenic plants transformed with an expression vector including an expression cassette encoding an AKIP operably linked to a heterologous plant non-constitutive promoter are provided according to embodiments of the present invention. The transgenic plants are characterized by enhanced senescence.

According to one embodiment, a transgenic potato plant transformed with an expression vector including an expression cassette encoding an AKIP operably linked to a heterologous plant non-constitutive promoter is provided according to the present invention. According to one embodiment, a transgenic cotton plant transformed with an expression vector including an expression cassette encoding an AKIP operably linked to a heterologous plant non-constitutive promoter is provided according to the present invention. According to one embodiment, a transgenic tobacco plant transformed with an expression vector including an expression cassette encoding an AKIP operably linked to a heterologous plant non-constitutive promoter is provided according to the present invention.

According to one embodiment, a transgenic potato plant transformed with an expression vector including an expression cassette encoding an AKIP operably linked to a heterologous plant non-constitutive promoter is selected from the group consisting of: alfalfa, apple, apricot, asparagus, avocado, banana, barley, bean, blackberry, broccoli, cabbage, carrot, cauliflower, celery, cherry, chicory, clover, cucumber, eggplant, garlic, grape, hemp, lettuce, maize, mango, melon, miscanthus, nectarine, onion, papaya, pea, peach, pear, pepper, pineapple, plum, pumpkin, quince, radish, rapeseed, rice, raspberry, rye, soybean, sorghum, spinach, squash, strawberry, sugarcane, sugarbeet, sunflower, sweet potato, switchgrass, tobacco, tomato, turnip, wheat, zucchini, aster, basil, bay leaf, begonia, chives, chrysanthemum, cilantro, clover, delphinium, dill, eucalyptus, lavender, lemon grass, mint, oregano, parsley, rosemary, savory, sunflower, tarragon, thyme, and zinnia.

Host cells transformed with an expression vector including an expression cassette encoding an AKIP operably linked to a heterologous plant non-constitutive promoter are provided according to the present invention.

Plant parts and progeny derived from transgenic plants transformed with an expression vector including an expression cassette encoding an AKIP operably linked to a heterologous plant non-constitutive promoter are provided according to the present invention.

Methods of making a transgenic plant characterized by enhanced senescence, including introduction of an expression cassette encoding an AKIP operably linked to a heterologous plant non-constitutive promoter into a cell of a plant or a portion of the plant and generating a whole plant from the cell or the portion of the plant.

Transgenic plants including a plant expression vector including an expression cassette nucleic acid encoding an AKIP, wherein the nucleic acid encoding the AKIP is operably linked to a heterologous plant senescence-specific and/or cell type-specific promoter are provided by the present invention, wherein the transgenic plant is characterized by enhanced senescence. Transgenic plants including a plant expression vector including an expression cassette nucleic acid encoding an AKIP, wherein the nucleic acid encoding the AKIP is operably linked to a heterologous plant senescence-specific and/or guard-cell-specific promoter are provided by the present invention, wherein the transgenic plant is characterized by enhanced senescence

Methods of harvesting a plant or a useful portion of a plant are provided according to the present invention which include non-constitutively increasing expression of an AKIP in the plant. According to preferred methods and compositions of the present invention, non-constitutively increasing expression of an AKIP in the plant includes specifically increasing of an AKIP in the plant during the developmental stage of senescence, wherein expression of the AKIP is not increased in the plant prior to the developmental stage of senescence.

According to embodiments of the present invention, increasing expression of an AKIP in the plant includes expression of an AKIP by an expression cassette in the plant, the expression cassette comprising a nucleic acid encoding the AKIP, the nucleic acid operably linked to a heterologous plant developmental stage-specific and/or cell type-specific promoter, wherein expression of the AKIP promotes senescence and wherein the plant is characterized by enhances senescence.

According to embodiments of the present invention, increasing expression of an AKIP in the plant includes expression of an AKIP by an expression cassette in the plant, the expression cassette comprising a nucleic acid encoding the AKIP, the nucleic acid operably linked to a heterologous plant senescence-specific and/or cell type-specific promoter, wherein expression of the AKIP promotes senescence and wherein the plant is characterized by enhances senescence.

Optionally, methods of harvesting a plant or a useful portion of a plant are provided according to the present invention which include non-constitutively increasing endogenous expression of an AKIP in the plant.

Methods of harvesting a plant or a useful portion of a plant are provided according to the present invention wherein the plant is a potato plant. Methods of harvesting a plant or a useful portion of a plant are provided according to the present invention wherein the plant is a cotton plant. Methods of harvesting a plant or a useful portion of a plant are provided according to the present invention wherein the plant is a tobacco plant.

Methods of harvesting a plant or a useful portion of a plant are provided according to the present invention wherein the plant is is selected from the group consisting of alfalfa, apple, apricot, asparagus, avocado, banana, barley, bean, blackberry, broccoli, cabbage, carrot, cauliflower, celery, cherry, chicory, clover, cucumber, eggplant, garlic, grape, hemp, lettuce, maize, mango, melon, miscanthus, nectarine, onion, papaya, pea, peach, pear, pepper, pineapple, plum, pumpkin, quince, radish, rapeseed, rice, raspberry, rye, soybean, sorghum, spinach, squash, strawberry, sugarcane, sugarbeet, sunflower, sweet potato, switchgrass, tomato, turnip, wheat, zucchini, aster, basil, bay leaf, begonia, chives, chrysanthemum, cilantro, clover, delphinium, dill, eucalyptus, lavender, lemon grass, mint, oregano, parsley, rosemary, savory, sunflower, tarragon, thyme, and zinnia.

Methods of harvesting a plant or a useful portion of a plant are provided according to the present invention wherein the plant is wherein expression of the AKIP promotes senescence manifested by drying of the plant or portion of the plant.

Methods of harvesting a plant or a useful portion of a plant are provided according to the present invention wherein the plant is wherein expression of the AKIP promotes senescence manifested by defoliation of the plant.

In preferred embodiments, plants are provided which are characterized by increased expression of an AKIP during senescence and not prior to senescence, compared to a wild-type plant.

### DETAILED DESCRIPTION OF THE INVENTION

Methods and compositions are provided according to embodiments of the present invention for promoting senescence in a plant. Methods and compositions of the present invention have utility, for example, to promote senescence in a crop plant such that non-crop portions of the plant interfere less with harvest of the useful part of the crop plant. Methods and compositions of the present invention also have utility, for example, to promote senescence in a crop plant such that the useful portion of the plant senesces more rapidly, when such senescence is the desired outcome, such as in drying of tobacco leaves or production of dried herbs.

Methods and compositions described herein allow for reducing or eliminating application of chemical defoliants and/or dessicants to plants to promote senescence.

In embodiments of the present invention, transgenic plants are provided characterized by increased expression of an AKIP protein during senescence compared to a similar wild-type plant. The increased expression of an AKIP protein during senescence promotes senescence such that, for example, the time to onset of senescence is shorter than in a wild-type plant and/or the time from onset of senescence to death is accelerated. AKIP is increased preferentially in selected cell-types or in all cells of the genetically modified plants depending on the characteristics of the expression construct used to generate the genetically modified plants.

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Agrobacterium-Mediated Plant Transformation: the Biology behind the “Gene-Jockeying” Tool: Microbiology and Molecular Biology Reviews, 2003, 67(1):16-37; Maliga, P., Methods in Plant Molecular Biology, Cold Spring Harbor Laboratory Press, New York, 1995; Weissbach, A. and Weissbach, H. Methods for Plant Molecular Biology, Academic Press, 1988; Jackson, J. F. and Linskens, H. F., Genetic Transformation of Plants, Molecular Methods of Plant Analysis, Springer, 2003; and Dashek, W. V., Methods in Plant Biochemistry and Molecular Biology, CRC Press, 1997.

Expression cassettes are provided according to embodiments of the present invention which include a recombinant nucleic acid including a nucleic acid encoding an AKIP protein operably linked to a heterologous non-constitutive plant promoter. The nucleic acid sequence encoding an AKIP may also be operably linked to one or more additional regulatory nucleic acid sequences which facilitates expression of the AKIP in an appropriate host cell. An expression cassette of the present invention can be generated using molecular biology methods or chemical synthetic techniques using well-known methodology.

The terms “nucleic acid” and “nucleic acid sequence” refer to DNA or RNA that is linear or circular, single-stranded or double-stranded.

The term “recombinant nucleic acid” refers to a nucleic acid which is altered, rearranged or modified by genetic engineering methods employed by a human.

The term “encoding” with respect to a nucleic acid sequence refers to a nucleic acid including the genetic information for translation of the nucleic acid sequence into a specified protein.

The term “operably linked” refers to a nucleic acid in functional relationship with a second nucleic acid. A regulatory nucleic acid sequence operably linked to a nucleic acid sequence encoding an AKIP facilitates expression of the nucleic acid sequence encoding AKIP in a host cell. A regulatory nucleic acid sequence is illustratively a promoter, transfer DNA (T-DNA), an enhancer, a DNA and/or RNA polymerase binding site, a ribosomal binding site, a polyadenylation signal, a transcription start site, a transcription termination site or an internal ribosome entry site (TRES).

The term “expression” as used herein refers to transcription of a DNA sequence to produce a corresponding mRNA. The term “expression” is also used herein to refer to translation of the mRNA to produce the corresponding protein.

The term “AKIP” refers to plant proteins known as “abscisic-acid-activated protein kinase-interacting proteins,” see Li, J. et al., Nature, 418:793-797, 2002 and WO 2004/013295. AKIPs are characterized by two conserved functional domains which are RNA-recognition motifs, designated RRM1 and RRM2.

The term “AKIP” encompasses Arabidopsis thaliana (At) AKIPs known as At AKIP1, At AKIP2 and At AKIP3. Amino acid sequences of At AKIP1, At AKIP2 and At AKIP3 are shown herein as SEQ ID Nos. 6, 8 and 10, respectively. Nucleic acid sequences encoding At AKIP1, At AKIP2 and At AKIP3 proteins are shown herein as SEQ ID Nos. 5, 7 and 9, respectively.

RRM1 and RRM2, of At AKIP1 are shown herein as SEQ ID Nos. 21 and 26, respectively. RRM1 and RRM2, of At AKIP2 are shown herein as SEQ ID Nos. 22 and 27, respectively. RRM1 and RRM2, of At AKIP3 are shown herein as SEQ ID Nos. 23 and 28, respectively.

The term “AKIP” further encompasses homologues and variants of At AKIP1, At AKIP2 and At AKIP3.

The term “AKIP homologue” refers to a protein characterized by an amino acid sequence substantially similar to the amino acid sequence of At AKIP1, At AKIP2 or At AKIP3 and which has substantially similar functional properties compared to At AKIP1, At AKIP2 or At AKIP3. The term “substantially similar amino acid sequence” and grammatical equivalents refers to an amino acid sequence having at least 30% or more identity to a reference AKIP amino acid sequence. Plant homologues of At AKIP1, At AKIP2 or At AKIP3 have at least 30%, at least 40%, or at least 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, 99%, or greater, amino acid sequence identity to At AKIP1, At AKIP2 or At AKIP3.

Percent identity is determined by comparison of amino acid or nucleic acid sequences, including a reference AKIP amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X 100%). The two sequences compared are generally the same length or nearly the same length.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP. BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present invention. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al. 1997, Nucleic Acids Res. 25:3389 3402. Alternatively, PSI BLAST is used to perform an iterated search which detects distant relationships between molecules (Id.). When utilizing BLAST, Gapped BLAST, and PSI Blast programs, the default parameters of the respective programs (e.g., of XBLAST and NBLAST) are used (see, e.g., the NCBI website). Another preferred, non limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

When comparing a reference AKIP to a putative AKIP homologue, amino acid similarity may be considered in addition to identity of amino acids at corresponding positions in an amino acid sequence. “Amino acid similarity” refers to amino acid identity and conservative amino acid substitutions in a putative AKIP homologue compared to the corresponding amino acid positions in a reference AKIP. Plant homologues of At AKIP1, At AKIP2 or At AKIP3 have at least 30% or greater, amino acid sequence identity to At AKIP1, At AKIP2 or At AKIP3 and at least 60%. 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, 99%, or greater amino acid similarity.

In preferred embodiments, plant homologues of At AKIP1, At AKIP2 or At AKIP3 are characterized by two RNA-recognition motifs wherein each RNA-recognition motif independently has at least 60% or greater identity and at least 70% or greater amino acid similarity to one of the RNA-recognition motifs present in a reference At AKIP. In further preferred embodiments, plant homologues of At AKIP1, At AKIP2 or At AKIP3 are characterized by two RNA-recognition motifs wherein each RNA-recognition motif independently has 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, 99%, or greater, amino acid identity and at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or greater, amino acid similarity to one of the RNA-recognition motifs present in a reference At AMP, SEQ ID Nos. 21, 22, 23, 26, 27 and 28.

The term “AKIP” encompasses potato AKIPs known as StAKIP1/2 and StAKIP3. Potato AKIPs are examples of plant Arabidopsis AKIP homologues characterized by amino acid sequences substantially similar to Arabidopsis AKIP amino acid sequences. The amino acid sequence of potato AKIP1/2, also called StAKIP1/2 herein is shown as SEQ ID No. 2 and the corresponding nucleic acid encoding StAKIP1/2 is shown as SEQ ID No.1. The amino acid sequence of potato AKIP3, also called StAKIP3 herein is shown as SEQ ID No. 4 and the corresponding nucleic acid encoding StAKIP3 is shown as SEQ ID No.3.

As for other AKIPS, potato AKIPs have two RNA-recognition motifs, RRM1 and RRM2. RRM1 and RRM2 of St AKIP1/2 are shown herein as SEQ ID Nos. 24 and 29, respectively. RRM1 and RRM2 of St AKIP3 are shown herein as SEQ ID Nos. 25 and 30, respectively.

In preferred embodiments, plant homologues of St AKIP1/2 or St AKIP3 are characterized by two RNA-recognition motifs wherein each RNA-recognition motif has 60% or greater identity to one of the RNA-recognition motifs present in a reference St AKIP. In further preferred embodiments, plant homologues of St AKIP1/2 or St AKIP3 are characterized by two RNA-recognition motifs wherein each RNA-recognition motif independently has 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, 99%, or greater, amino acid identity and at least 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, 99%, or greater, amino acid similarity to one of the RNA-recognition motifs present in a reference St AKIP, SEQ ID Nos. 24, 25, 29 and 30.

TABLE 1
Identities of RRM regions of StAKIPs vs At AKIPs (Percentage in parenthesis denotes similarities).

| | | At AKIP1 | | At AKIP2 | | At AKIP3 | |
|---|---|---|---|---|---|---|---|
| | | RRM 1 | RRM 2 | RRM 1 | RRM 2 | RRM 1 | RRM 2 |
| StAKIP1/2 | RRM 1 | 78% (83%) | 33% (58%) | 76% (83%) | 33% (56%) | 55% (73%) | 36% (61%) |
| | RRM 2 | 41% (63%) | 78% (91%) | 36% (63%) | 75% (93%) | 40% (61%) | 53% (72%) |
| StAKIP3 | RRM 1 | 44% (67%) | 33% (57%) | 45% (64%) | 30% (59%) | 62% (83%) | 24% (55%) |
| | RRM 2 | 43% (65%) | 56% (71%) | 36% (65%) | 56% (72%) | 36% (62%) | 63% (83%) |

An AKIP homologue is encoded by a nucleic acid sequence having substantial similarity to nucleic acid sequences encoding an AKIP disclosed herein. The term “substantially similar nucleic acid sequence” and grammatical equivalents refers to a nucleic acid sequence having 70% or more identity to a reference nucleic acid sequence. A nucleic acid sequence having substantial similarity to a nucleic acid sequence encoding At AKIP1, At AKIP2, At AKIP3, St AKIP1/2 or St AKIP3 has at least 70%, at least 75%, or at least 85%, 90%, 91%, 92%, 93%, 94%, 95%, 96%, 97%, 98%, 99%, or greater, nucleic acid sequence identity to nucleic acid sequences encoding At AKIP1, At AKIP2 or At AKIP3, St AKIP1/2 or St AKIP3

The term “AKIP” encompasses variants of At AKIP1, At AKIP2, At AKIP3, StAKIP1/2, StAKIP3 and homologues thereof. The term “AKIP” further encompasses functionally active AKIP fragments.

As used herein, the term “AKIP variant” refers to either a naturally occurring or a recombinantly prepared variation of a reference nucleic acid or protein.

Mutations can be introduced using standard molecular biology techniques, such as site-directed mutagenesis and PCR-mediated mutagenesis, to produce variants.

One of skill in the art will recognize that one or more amino acid mutations can be introduced without altering the functional properties of AKIP proteins. For example, one or more amino acid substitutions, additions, or deletions can be made without altering the functional properties of AKIP proteins.

Conservative amino acid substitutions can be made in AKIP proteins to produce variants. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, glycine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, praline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

It will be appreciated by those of skill in the art that due to the degenerate nature of the genetic code, multiple nucleic acid sequences can encode a particular AKIP, and that such alternate nucleic acids may be included in an expression construct and included in transgenic plants of the present invention.

In particular embodiments, a substantially similar nucleic acid sequence is characterized as having a complementary nucleic acid sequence capable of hybridizing to a nucleic acid sequence encoding At AKIP1, At AKIP2, At AKIP3, St AKIP1/2 or St AKIP3 under high stringency hybridization conditions.

The terms “hybridizing” and “hybridization” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. High stringency hybridization conditions are those which only allow hybridization of highly complementary nucleic acids. Determination of stringent hybridization conditions is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002.

An example of highly stringent hybridization conditions are: hybridization in a solution containing 6×SSC, 5× Denhardt's solution, 30% formamide, and 100 micrograms/ml denatured salmon sperm DNA at 37° C. overnight followed by washing in a solution of 0.1×SSC and 0.1% SDS at 60° C. for 15 minutes.

Because conserved RNA recognition motifs of AKIPs are well conserved between plant species, homologues and variants can be isolated using degenerate primers designed to hybridize with nucleic acids which are substantially similar to reference nucleic acids encoding an AKIP. PCR amplification of cDNA of a target plant using degenerate primers is performed according to standard PCR procedures to isolate putative AKIP homologues and variants in the target species. A putative homologue or variant is identified as an AKIP homologue or variant, for example, by nucleic acid and amino acid sequence analysis.

AKIP homologues and variants are characterized by conserved functional properties compared to naturally occurring AKIPs such as At AKIP1, At AKIP2, At AKIP3, StAKIP1/2 and StAKIP3. Thus, for example, AKIP homologues and variants retain the ability to promote senescence when expressed in a plant host cell such as in a host cell in a plant or plant part. Functional characteristics of the putative homologue or variant can be assayed, for example, transient transformation of leaf cells of an annual plant to detect promotion of senescence. Assays for AKIP activity to promote senescence, but are not limited to, transformation of a host cell with an expression cassette encoding a putative AKIP homologue or variant, followed by measurement of cell death, chlorophyll content, measurement of ethylene production and/or analysis of expression of senescence-associated genes in the host cell, where decreased chlorophyll content, increased ethylene production and/or increased expression of senescence—associated genes compared to control host cells is indicative of conserved AKIP functional properties of an AKIP homologue or variant. Assays for measurement of cell viability, chlorophyll content, measurement of ethylene production and/or analysis of expression of senescence-associated genes in the host cell are known in the art and are described herein.

A nucleic acid sequence encoding an AKIP homologue or variant can be isolated from a monocot or dicot including, but not limited to, alfalfa, apple, apricot, asparagus, avocado, banana, barley, bean, blackberry, broccoli, cabbage, carrot, cauliflower, celery, cherry, chicory, clover, cotton, cucumber, eggplant, garlic, grape, hemp, lettuce, maize, mango, melon, miscanthus, nectarine, onion, papaya, pea, peach, pear, pepper, pineapple, plum, potato, pumpkin, quince, radish, rapeseed, rice, raspberry, rye, soybean, sorghum, spinach, squash, strawberry, sugarcane, sugarbeet, sunflower, sweet potato, switchgrass, tobacco, tomato, turnip, wheat, zucchini, as well as woody plants such as coniferous and deciduous trees.

Expression cassettes are provided according to embodiments of the present invention which include a recombinant nucleic acid including a nucleic acid encoding an AKIP protein operably linked to a heterologous non-constitutive plant promoter. The nucleic acid sequence encoding an AKIP may also be operably linked to one or more additional regulatory nucleic acid sequences which facilitates expression of the AKIP in an appropriate host cell. Such regulatory nucleic acid sequences illustratively include an enhancer, transferred DNA (T-DNA), a splicing signal, a transcription start site, a transcription termination signal, a polyadenylation signal and an internal ribosome entry site (IRES).

Exemplary promoters are constitutively active promoters, inducible promoters and cell-type specific promoters.

The term “promoter” refers to non-transcribed cis-regulatory DNA elements which confer control over transcription of an operably linked nucleic acid sequence. Promoters include cis-regulatory elements that define functional elements such as the transcription initiation site and transcription factor binding sites. Promoters are generally located in the 5′ non-coding region of a gene and can be isolated for insertion into an expression cassette using standard recombinant techniques. Promoters can be derived entirely from a single gene or can be chimeric, having portions derived from more than one gene.

As will be recognized by one of skill in the art, a constitutive promoter is an unregulated promoter that allows continual and wide-spread expression of an operably linked nucleic acid. It is an aspect of the present invention that constitutive expression of an exogenous AKIP in a plant causes nearly immediate death of the plant. Thus, a heterologous non-constitutive promoter is preferably included in expression cassettes of the present invention to confer control over the expression of the operably linked nucleic acid encoding an AKIP. The term “heterologous non-constitutive promoter” refers to a non-constitutive promoter which is not a naturally occurring promoter of the operably linked nucleic acid sequence encoding an AKIP.

Expression constructs of the present invention include an heterologous non-constitutive promoter which confers expression of the operably linked nucleic acid encoding an AKIP when desired, such as at a desired plant developmental stage, in a particular part of the plant, in response to particular environmental conditions such as a particular temperature range, and/or by drought stress. In preferred embodiments, the promoter is a heterologous plant developmental stage-specific and/or cell type-specific promoter.

In further preferred embodiments, the promoter is a heterologous plant senescence-specific promoter. A senescence-specific promoter is more active in promoting expression of an operably linked nucleic acid in senescing plant cells than in the same cells during other plant developmental stages.

The term “heterologous plant senescence-specific promoter” encompasses promoters of plant senescence activated genes (SAG). Plant senescence is characterized by cell death and preferential up-regulation of senescence activated genes. SAGs induced during senescence include SAG13 (At2g29350); SAG14 (At5g20230); SAG15 (At5g51070); SAG101 (At5g14930); SIRK (At2g19190); WRKY6 (At1g62300); WRKY53 (At4g23810); and WRKY70 (At3g56400) as described Miller et al., Plant Physiol., 120:1015-1024, 1999.

SAGs further include SAG12 (At5g45890), which encodes a cysteine protease in leaves undergoing age-related senescence that are showing signs of chlorosis, as described in Lohman et al., Physiol. Plantarum, 92:322-328, 1994; and Nob & Amasino, Plant Mol. Biol., 41:181-194, 1999. SAGs include those identified in Gepstein et al., Plant J., 36:629-642, 2003. The promoter of any of these or other SAGs can be included in an expression construct of the present invention.

As will be recognized by the skilled artisan, the 5′ non-coding region of a gene can be isolated and used in its entirety as a promoter in an expression cassette. Alternatively, a portion of the 5′ non-coding region can be isolated and inserted in an expression cassette. In general, about 1000 bp of the 5′ non-coding region of a senescence activated gene is included in an expression cassette to confer senescence activated expression of the operably linked nucleic acid encoding a heterologous AKIP. Optionally, a portion of the 5′ non-coding region of a senescence activated gene containing a minimal amount of the 5′ non-coding region needed to confer senescence activated expression of the operably linked nucleic acid encoding a heterologous AKIP. Assays described herein can be used to determine the ability of a designated portion of the 5′ non-coding region of a senescence activated gene to confer senescence activated expression of the operably linked nucleic acid encoding a heterologous AKIP.

Thus, plant senescence-specific promoters included in expression cassettes of the present invention can be the 5′ non-coding region or a portion of the 5′ coding region which confers senescence activated expression of an operably linked nucleic acid encoding a heterologous AKIP of any senescence activated plant gene, including, but not limited to, SPG31; SAG2 (At5g60360); SAG12 (At5g45890), SAG13 (At2g29350); SAG14 (At5g20230); SAG15 (At5g51070); SAG101 (At5g14930); SIRK (At2g19190); WRKY6 (At1g62300); WRKY53 (At4g23810); and WRKY70 (At3g56400).

An exemplary included SAG promoter is the promoter of SAG12 (At5g45890). SAG12 encodes a cysteine protease in leaves undergoing age-related senescence that are showing signs of chlorosis, as described in Lohman et al., Physiol. Plantarum, 92:322-328, 1994; and Nob & Amasino, Plant Mol. Biol., 41:181-194, 1999. The promoter of SAG12, designated pSAG12 herein, is shown as SEQ ID No. 11.

An exemplary included SAG promoter is the promoter of SAG2. SAG2 is a member of a senescence-associated gene family and its expression upon onset of senescence is strong, as described in Grbic, Physiologia Plantarum 119:263-269. 2003. The promoter of SAG2, designated pSAG2 herein, is shown as SEQ ID No.19.

Sweet potato SPG31 gene is homologous to SAG12 gene and shows very strong up-regulation upon onset of senescence as described in Chen et al., Plant Cell Physiol. 43:984-91, 2002. The SPG31 gene is strongly expressed in leaves, indicating that the SPG31 promoter drives expression that is essentially leaf-specific. Thus, the promoter of the SPG31 gene, shown herein as SEQ ID No. 12, drives both leaf-specific and senescence-specific gene expression.

The term “heterologous non-constitutive promoter” encompasses promoters of plant cell-type specific genes.

A cell-type specific promoter promotes expression of an operably linked nucleic acid preferentially in a subset of cells of a plant. For example, a guard cell specific promoter promotes expression of an operably linked nucleic acid preferentially in guard cells. The term “heterologous non-constitutive promoter” encompasses promoters of guard cell-specific genes_{—} An example of a guard-cell specific promoter is pGC1, shown herein as SEQ ID No. 13.

Promoter homologues and promoter variants can be included in an expression cassette of the present invention. The term “promoter homologue” refers to a heterologous non-constitutive promoter which has substantially similar functional properties to confer senescence-specific and/or cell-type-specific expression on a heterologous operably linked nucleic acid compared to those disclosed herein, such as SEQ ID No. 11, SEQ ID No. 12, SEQ ID No. 13 and SEQ ID No. 19.

One of skill in the art will recognize that one or more nucleic acid mutations can be introduced without altering the functional properties of a given promoter. Mutations can be introduced using standard molecular biology techniques, such as site-directed mutagenesis and PCR-mediated mutagenesis, to produce promoter variants. As used herein, the term “promoter variant” refers to either a naturally occurring or a recombinantly prepared variation of a reference promoter, such as SEQ ID No. 11, SEQ ID No. 12, SEQ ID No. 13 and SEQ ID No. 19.

An expression cassette of the present invention can be incorporated into a vector, such as an expression vector and/or cloning vector. The term “vector” refers to a recombinant nucleic acid vehicle for transfer of a nucleic acid.

Expression vectors for insertion of an expression cassette and expression of a recombinant nucleic acid in an appropriate plant host cell are well-known in the art. Exemplary vectors are plasmids, cosmids, viruses and bacteriophages. Particular vectors are known in the art and one of skill in the art will recognize an appropriate vector for a specific purpose. Plant expression vectors are described, for example, in Maliga, P., Methods in Plant Molecular Biology, Cold Spring Harbor Laboratory Press, New York, 1995; Weissbach, A. and Weissbach, H. Methods for Plant Molecular Biology, Academic Press, 1988; Jackson, J. F. and Linskens, H. F., Genetic Transformation of Plants, Molecular Methods of Plant Analysis, Springer, 2003; and Dashek. W. V., Methods in Plant Biochemistry and Molecular Biology, CRC Press, 1997.

An expression cassette provided according to embodiments of the present invention is included in a recombinant nucleic acid vector and used to transform a host plant cell to generate a transgenic plant.

The term “transgenic plant” refers to a plant having one or more, or all, plant cells that contain an expression cassette including a nucleic acid encoding an AKIP, the nucleic acid encoding an AKIP operably linked to a heterologous non-constitutive promoter. The expression cassette can be stably integrated into the genome or may be extrachromasomal.

A transgenic plant expresses AKIP encoded by a nucleic acid of the expression cassette which would not otherwise be expressed in the plant or expresses the encoded AKIP at a different level, in different cells, at a different developmental stage or in an otherwise different pattern than in a wild-type plant. Transgenic plants of the present invention are provided which express during senescence 1) an increased amount of an AKIP typically present in a similar wild-type plant and/or 2) an AKIP which is not present in a similar wild-type plant.

The term transgenic plant also refers to a transgenic plant part. A transgenic plant of the present invention is a direct transformant, that is, an expression cassette is introduced directly into the plant, or a transgenic plant can be progeny of a direct transformant.

Progeny of a transgenic plant described herein is encompassed by the present invention. The term “progeny” referring to a transgenic plant describes a transgenic plant of the present invention which is derived from a direct transformant, such as an F1 generation plant. The progeny contains one or more, or all, plant cells having the inherited expression cassette encoding an AKIP. Progeny can have one or more mutations or changes in copy number of the nucleic acid encoding the AKIP which occur by deliberate genetic manipulation or by natural causes. Such mutations and changes are considered to be within the scope of the present invention as long as the protein expressed from the inherited expression cassette is substantially similar to a reference AKIP, such as At AKIP1 At AKIP2, At AKIP3, St AKIP1/2 or St AKIP3.

The term “wild-type” is used to refer to a plant or cell which is not modified using a composition or method of the present invention and which therefore does not contain a recombinant expression cassette encoding AKIP.

The terms “transform” and “transformation” refers to a process of introducing an expression construct into a recipient host. The host cell can be a microorganism or a plant cell; including a plant cell in vitro, a plant cell in a plant, i.e. in planta, and/or a plant cell in a plant part. Microorganisms are host cells for e.g. propagation and amplification of the expression cassette and/or expression vector.

The term “plant part” refers to any portion of a plant, including, but not limited to, seeds, stems, embryos, pollen, leaves, tubers, protoplasts, calli, roots, stamens, ovules, meristematic regions, gametophytes, sporophytes and microspores.

Methods of transformation are well-known in the art and include, but are not limited to, Agrobacterium-mediated transformation, electroporation, particle accelerated transformation also known as “gene gun” technology, liposome-mediated transformation, microinjection, polyethylene glycol mediated transformation, heat shock mediated transformation and virus-mediated transformation. Examples of methods of transformation are described in Agrobacterium-Mediated Plant Transformation: the Biology behind the “Gene-Jockeying” Tool: Microbiology and Molecular Biology Reviews, March 2003, p. 16-37, Vol. 67, No. 1; Maliga, P., Methods in Plant Molecular Biology, Cold Spring Harbor Laboratory Press, New York, 1995; Weissbach, A. and Weissbach, H. Methods for Plant Molecular Biology, Academic Press, 1988; Jackson, J. F. and Linskens, H. F., Genetic Transformation of Plants, Molecular Methods of Plant Analysis, Springer, 2003; and Dashek, W. V., Methods in Plant Biochemistry and Molecular Biology, CRC Press, 1997. Any of these or other effective transformation methods can be used to generate transgenic plants expressing AKIP.

The development, regeneration and cultivation of plants containing an expression construct from a cell, plant part and/or plant transformed with the expression construct is well-known in the art as exemplified in Agrobacterium-Mediated Plant Transformation: the Biology behind the “Gene-Jockeying” Tool: Microbiology and Molecular Biology Reviews, March 2003, p. 16-37, Vol. 67, No. 1; Maliga, P., Methods in Plant Molecular Biology, Cold Spring Harbor Laboratory Press, New York, 1995; Weissbach, A. and Weissbach, H. Methods for Plant Molecular Biology, Academic Press, 1988; Jackson, J. F. and Linskens, H. F., Genetic Transformation of Plants, Molecular Methods of Plant Analysis, Springer, 2003; and Dashek, W. V., Methods in Plant Biochemistry and Molecular Biology, CRC Press, 1997.

In general, transformed cells, embryos or seeds are selected and cultured to produce rooted transgenic plantlets which can then be planted in soil or another suitable plant growth medium.

One of skill in the art will recognize that individual transformation events will result in different levels of expression of a transgene, as well as different patterns of expression, such as temporal or spatial patterns, in a cell or organism. Routine screening of multiple transformation events is performed to obtain lines having a desired level of expression of the transgene and/or a desired pattern of expression. Such routine screening is accomplished using well-known techniques such as Southern blot, Northern blot, Western blot and/or phenotypic analysis for a desired characteristic.

Development and regeneration of transformed plants is well-known in the art. Regenerated transgenic plants are preferably self-pollinated to produce homozygous transgenic plants. Optionally, a regenerated transgenic plant is pollinated by a plant which is not a transgenic plant of the present invention, or, pollen from a regenerated transgenic plant is used to pollinate a plant which is not a transgenic plant of the present invention. Development and regeneration of transgenic plants from a transformed plant part are also well-known in the art.

Any monocot or dicot plant is transformed to produce a transgenic plant of the present invention including, but not limited to, alfalfa, apple, apricot, asparagus, avocado, banana, barley, bean, blackberry, broccoli, cabbage, carrot, cauliflower, celery, cherry, chicory, clover, cotton, cucumber, eggplant, garlic, grape, hemp, lettuce, maize, mango, melon, miscanthus, nectarine, onion, papaya, pea, peach, pear, pepper, pineapple, plum, potato, pumpkin, quince, radish, rapeseed, rice, raspberry, rye, soybean, sorghum, spinach, squash, strawberry, sugarcane, sugarbeet, sunflower, sweet potato, switchgrass, tobacco, tomato, turnip, wheat, zucchini, as well as woody plants such as coniferous and deciduous trees.

In addition to transgenic crop plants, compositions and methods of the present invention have utility to produce dried plants and plant parts and transgenic plants traditionally used to provide decorative flowers and herbs are provided according to the present invention. Examples of such plants provided according to the present invention include, but are not limited to, transgenic aster, basil, bay leaf, begonia, chives, chrysanthemum, cilantro, clover, delphinium, dill, eucalyptus, lavender, lemon grass, mint, oregano, parsley, rosemary, savory, sunflower, tarragon, thyme, and zinnia.

According to one embodiment, a tuber producing crop plant is transformed to produce a transgenic plant of the present invention. For example, a transgenic potato plant of the present invention is transformed with an expression cassette including a nucleic acid encoding an AKIP, the nucleic acid encoding an AKIP operably linked to a heterologous non-constitutive promoter, producing a transgenic potato plant characterized by enhanced senescence such that foliage becomes yellow, dries and drops within a shorter time after onset of senescence compared to a wild-type or control potato plant. The transgenic potato plant requires little or no application of chemical defoliant prior to harvest of the tuber compared to wild-type potato plants.

In one non-limiting example, a tuber producing crop plant is transformed to produce a transgenic plant of the present invention. For example, a potato plant is transformed with an expression cassette including a nucleic acid encoding an AKIP, the nucleic acid encoding an AKIP operably linked to a heterologous non-constitutive promoter, producing a transgenic potato plant of the present invention characterized by enhanced senescence such that foliage becomes yellow, dries and drops within a shorter time after onset of senescence compared to a wild-type or control potato plant. The transgenic potato plant requires little or no application of chemical defoliant prior to harvest of the tuber compared to wild-type potato plants.

In one non-limiting example, a soybean plant is transformed to produce a transgenic plant of the present invention. The soybean plant is transformed with an expression cassette including a nucleic acid encoding an AKIP, the nucleic acid encoding an AKIP operably linked to a heterologous non-constitutive promoter, producing a transgenic soybean plant characterized by enhanced senescence such that foliage becomes yellow, dries and drops within a shorter time after onset of senescence compared to a wild-type or control soybean plant. The transgenic soybean plant requires little or no application of chemical defoliant prior to harvest of the soybeans compared to wild-type soybean plants.

In one non-limiting example, a cotton plant is transformed to produce a transgenic plant of the present invention. The cotton plant is transformed with an expression cassette including a nucleic acid encoding an AKIP, the nucleic acid encoding an AKIP operably linked to a heterologous non-constitutive promoter, producing a transgenic cotton plant characterized by enhanced senescence such that foliage becomes yellow, dries and drops within a shorter time after onset of senescence compared to a wild-type or control cotton plant. The transgenic cotton plant requires little or no application of chemical defoliant prior to harvest of the cotton compared to wild-type cotton plants.

Identification of Transgenic Plants Characterized by Enhanced Senescence

Transgenic plants of the present invention including an expression cassette encoding an AKIP operably linked to a heterologous promoter display enhanced senescence due to promotion of senescence by expression of the AKIP. Enhanced senescence in transgenic plants of the present invention is detected as, for example, shorter time to onset of senescence compared to a similar wild-type plant and/or shorter time from onset of senescence to death compared to a similar wild-type plant.

Identification of enhanced senescence in transgenic plants can be qualitative or quantitative. For example, time to onset of senescence during development of a transgenic plant is compared to a similar wild-type plant. Signs of onset of senescence include yellowing of leaves. Quantitative assessment of enhanced senescence phenotype in transgenic plants is performed, for example, by measurement of loss of cell viability, chlorophyll content, measurement of ethylene production and/or analysis of expression of senescence-associated genes, compared to a similar wild-type plant.

Leaf yellowing is caused by chlorophyll breakdown and is a characteristic symptom of senescence (Miller et al., Plant Physiol 120:1015-1024, 1999). Therefore, chlorophyll content is a good indicator of the degree of senescence. To measure chlorophyll content, chlorophyll is extracted using dimethyl formamide (DMF) from freeze-dried leaf tissues and chlorophyll-containing DMF solutions are analyzed by their absorbance at 646.8 and 663.8 nm using a spectrophotometer. Chlorophyll a+b concentration in nmol mL⁻¹ is calculated as 19.43A^{646.8}+8.05 A^{663.8} after subtraction of absorbance at 750 nm, following the formula of Porra et al. 1989. Biochem et Biophys Acta 975: 384-394.

Ethylene production is another indicator of the degree of senescence. In Arabidopsis transgenic plants, it is noted that transcript levels of ACS2 and ACS6, which encode 1-aminocyclopropane-1-carboxylic acid synthase, the key enzyme implicates in ethylene biosynthesis, were shown to be enhanced 24 h and 48 h after induction of UBA2 overexpression (Kim et al., 2008 New Phytol. 180:57-70. Transgenic plants are used for ethylene measurement following the method described by Kim et al., 2008 New Phytol. 180:57-70. Three leaves with petioles are excised and incubated for 1.5 h under light in a sealed vial (20 ml) containing 1 ml of water. A headspace sample (1 ml) is drawn from each vial and ethylene levels are analysed using a gas chromatograph (model 5840A; Hewlett Packard, Palo Alto, Calif., USA). Transgenic Arabidopsis plants overexpressing AKIPs were shown to have significantly higher ethylene production compared to control plants (Kim et al., 2008 New Phytol. 180:57-70).

The senescence program involves the preferential expression of SAGs, indicating that SAG gene expression can be used to quantify the degree of senescence. RT-PCR analyses are used to quantify SAG gene expression. In Arabidopsis, in parallel with UBA2-induced overexpression, increased expression of SAGs was observed, including SAG13, SAG14, SAG15, SAG101 as described in Kim et al., 2008 New Phytol. 180:57-70 and herein.

Increased expression of an endogenous AKIP is achieved by screening a mutant population of plants to identify a line with a mutated AKIP promoter conferring the desired expression characteristics.

Increased expression of an endogenous AKIP can also be achieved by homologous recombination to swap out the endogenous AKIP promoter for one with the desired characteristics.

Methods of promoting senescence in a plant are provided according to embodiments of the present invention which include increasing expression of an AKIP during senescence. In preferred embodiments, methods of promoting senescence in a plant include providing a transgenic plant including an expression cassette, wherein the expression cassette includes a nucleic acid sequence encoding an AKIP, the nucleic acid sequence encoding the AKIP operably linked to a heterologous non-constitutive plant promoter. In further preferred embodiments, methods of promoting senescence in a plant include providing a transgenic plant including an expression cassette, wherein the expression cassette includes a nucleic acid sequence encoding an AKIP, the nucleic acid sequence encoding the AKIP operably linked to a heterologous senescence-specific and/or guard cell-specific plant promoter. In still further preferred embodiments, methods of promoting senescence in a plant include providing a transgenic plant including an expression cassette, wherein the expression cassette includes a nucleic acid sequence encoding an AKIP, the nucleic acid sequence encoding the AKIP operably linked to a heterologous senescence-activated gene promoter and/or guard cell-specific plant promoter.

Methods of harvesting a crop are provided which promote senescence using reduced application of chemical defoliators or desiccants wherein the crop is a non-senescing portion of a transgenic plant described herein. Any monocot or dicot crop transgenic plant of the present invention is used including, but not limited to, alfalfa, apple, apricot, asparagus, avocado, banana, barley, bean, blackberry, broccoli, cabbage, carrot, cauliflower, celery, cherry, chicory, clover, cotton, cucumber, eggplant, garlic, grape, hemp, lettuce, maize, mango, melon, miscanthus, nectarine, onion, papaya, pea, peach, pear, pepper, pineapple, plum, potato, pumpkin, quince, radish, rapeseed, rice, raspberry, rye, soybean, sorghum, spinach, squash, strawberry, sugarcane, sugarbeet, sunflower, sweet potato, switchgrass, tobacco, tomato, turnip, wheat, zucchini, as well as woody plants such as coniferous and deciduous trees.

Embodiments of inventive compositions and methods are illustrated in the following examples. These examples are provided for illustrative purposes and are not considered limitations on the scope of inventive compositions and methods.

### EXAMPLES

### Example 1

Plant Material and Growth Conditions

Arabidopsis thaliana (L.) Heynh. ecotype Columbia (Col-0) is used in this example. Seeds are surface-sterilized, transferred to half-strength Murashige and Skoog (MS) medium described in Murashige & Skoog, Physiol. Plantarum, 15: 473-497, 1962), and transplanted to soil as described in Bove et al., Plant Mol. Biol., 67:71-88, 2008. Plants are grown to maturity under short-day conditions (8 h light:16 h dark cycle) at 22° C. at a light intensity of 120 μmol m⁻² s⁻¹. Five-week-old, fully expanded healthy leaves are used for all experiments, except where noted otherwise. Wild-type plants for expression analysis at different developmental stages are grown under long-day (16 h light:8 h dark cycle) conditions for 10 wk and leaves are harvested at 1-wk intervals.

Constructs of 2×35S-UBA2s, DEX-UBA2s and EST-UBA2s

Arabidopsis AKIPs are traditionally known by different nomenclature. Arabidopsis AKIP1 has been known as Arabidopsis UBA2a. Arabidopsis AKIP2 has been known as Arabidopsis UBA2b. Arabidopsis AKIP3 has been known as Arabidopsis UBA2c. Arabidopsis Genome Initiative locus identifiers for the UBA2 genes are: UBA2a (At3g56860), UBA2b (At2g41060) and UBA2c (At3g15010). Full-length cDNAs of UBA2a, UBA2b and UBA2c are cloned into the pCR-Blunt II-TOPO vector as described in Bove et al., Plant Molecular Biology 67:71-88, 2008. The coding regions of UBA2s are amplified by Pfx DNA polymerase (Invitrogen, Carlsbad, Calif., USA) from the plasmids pCR-Blunt-UBA2s with gene-specific oligonucleotides containing an Nde I site engineered (underlined) into the upstream primer and the C-terminal reverse primer as follows: UBA2a-NdeI, 5′-CATATGACAAAGAAGAGAAAGCTCGAA-3′ SEQ ID No. 31; UBA2a-C-ter, 5′-GGATACATGATACATTTAGTGACCCATG-3′ SEQ ID No. 32; UBA2b-NdeI, 5′-CATATGACAAAGAAGAGAAAGCTCGAA-3′ SEQ ID No. 33; UBA2b-C-ter, 5′-CCTGAGTGGCTAATCTAACGACCCATG-3′ SEQ ID No. 34; UBA2c-NdeI, 5′-CATATGGATATGATGAAGAAGCGTAAGC-3′ SEQ ID No. 35; UBA2c-C-ter, 5′-TTTTCAGTAGTTTGGTGGACCGTTGGG-3′ SEQ ID No. 36. The resulting UBA2 cDNAs are first cloned in-frame into an intermediate vector with a FLAG-epitope tag, and the Ω sequence from tobacco mosaic virus described in Gallie et al., Nucleic. Acids Res., 15:3257-3273, 1987. The UBA2 cDNAs with a FLAG tag coding sequence at their 5′ termini are placed behind the CaMV 2×35S promoter at the XhoI-SpeI restriction sites of a modified pBI121 binary vector. The UBA2 cDNAs are also introduced into the DEX-inducible (GVG) described in Aoyama & Chua, Plant J., 11:605-612, 1997and estradiol (EST)-inducible binary vector (XVE) described in Zuo et al., Plant J., 24:265-273, 2000, with XhoI and SpeI cloning sites. The constructs are confirmed by sequencing.

Agrobacterium tumefaciens-Mediated Transformation

The UBA2 constructs in the binary vectors are electroporated into Agrobacterium tumefaciens strain C58C1.

Stable transgenic Arabidopsis plants are generated using the floral dip method (Clough & Bent, Plant J., 16:735-743, 1998). The T1 transformants are selected in the presence of kanamycin and hygromycin for the 2×35S promoter and Dexamethasone (DEX)-inducible constructs, respectively. After selection, the seedlings are transplanted to soil for morphological observation and seed set (T1 for 2×35S promoter constructs; T2 and T3 for DEX-inducible constructs). To screen for transgenic lines expressing the DEX-inducible constructs, detached leaves of plants are sprayed with DEX (15 μM) to induce the expression of transgene, and the expression of transgene is analysed by western blot analysis with M2 anti-FLAG antibody (1:10 000 dilution; Sigma, St Louis, Mo., USA) 15 h later.

For the Agrobacterium-mediated transient expression assay, agrobacteria harboring the different constructs are grown overnight in Luria-Bertani (LB) medium containing 25 μg, ml⁻¹ of gentamycin and 50 μg ml⁻¹ of kanamycin, and 150 μM acetosyringone. Cells are collected by centrifugation (4000 g), resuspended to an optical density of 600 nm (0D600) of 1.0 in infiltration medium (10 mM 2-(N-morpholino) ethanesulfonic acid (MES), pH 5.6, 10 mM MgCl₂) with 150 μM acetosyringone, and infiltrated into leaves of 6-wk-old Nicotiana benthamiana plants. Expression of the transgenes is induced by spraying 10 μM 17-β-estradiol (EST) 40-48 h later.

UBA2-mYFP Constructs and Stable Transgenic Plants

For subcellular localization of UBA2-mYFP fusion proteins, the UBA2 cDNA inserts are fused in-frame with mYFP at their C-termini as described by Bove et al., Plant Mol. Biol., 67:71-88, 2008. A SalI-SpeI fragment from a pUBA2-mYFP clone for each of the 3 UBA2 cDNAs is introduced between XhoI-SpeI restriction sites in the DEX-inducible binary vector. The DEX inducible UBA2-mYFP constructs are transformed into Arabidopsis plants to generate stable transgenic plants as described above. Transgenic plants are screened by microscopic analysis at 15 h after DEX application in detached leaves from transgenic Arabidopsis plants. For mYFP image analysis, epidermal peels are taken from 5-wk-old Arabidopsis plants that had been treated with DEX 2 d previously and examined using a confocal LSM 510 Meta microscope (Zeiss) equipped with a ×20/0.75 planapochromat lens and a ×40/0.75 plan-apochromat water immersion lens.

Protein Extraction and Immunoblot Analysis

Total proteins are extracted from leaf tissue by grinding with a small plastic pestle in extraction buffer (100 mM N-2-hydroxyethylpiperazine-N′-2-ethanesulfonic acid (HEPES), pH 7.5, 5 mM ethylenediaminetetraacetic acid (EDTA), 5 mM EGTA, 10 mM dithiothreitol, 1 mM phenylmethylsulfonyl fluoride (PMSF), 5% glycerol). After centrifugation at 14 000 g for 40 min, supernatants, containing soluble protein, are transferred into clean tubes, quickly frozen in liquid nitrogen, and stored at −80° C. until analyses. Protein concentration is determined by the Bradford protein assay kit (Bio-Rad, Hercules, Calif., USA) with bovine serum albumin as a standard. For immunoblot analysis, 15 μg of total protein per lane are separated by electrophoresis on 10% sodium dodecyl sulfate (SDS)-polyacrylamide gels, and the proteins are transferred to nitrocellulose membranes (Schleicher and Schuell, PerkinElmer, Waltham, Mass., USA) by semidry electroblotting. After blocking for 2 h in 1× Tris-buffered saline (1× Tris buffered saline (TBS): 20 mM Tris-HCl, pH 7.5, 100 mM NaCl) containing 0.05% Tween 20 and 5% nonfat dried milk (Carnation) at room temperature, the membranes are incubated with M2 anti-FLAG antibody (1:10 000 dilution; Sigma). Following three washes with 1× TBS buffer containing 0.05% Tween 20, the membranes are incubated with horseradish peroxidase-conjugated secondary antibody (1:10 000 dilution; Pierce, Rockford, Ill., USA). The membranes are visualized using an enhanced chemiluminescence kit (Pierce) according to the manufacturer's instructions.

Plant Treatments and RNA Extraction

Five-week-old transgenic Arabidopsis plants are sprayed once with 15 μM of DEX and leaf discs are collected from the rosette leaves at the indicated time points using a cork borer (10 mm in diameter), quick frozen in liquid N₂, and stored at −80° C. until use. Total RNA is extracted from the frozen samples (eight leaf discs per assay) using the Plant RNeasy extraction kit (Qiagen, Valencia, Calif., USA) or extracted using Trizol (Invitrogen). To remove genomic DNA contamination, total RNA is treated with DNase I on column according to the manufacturer's protocol (Qiagen) or treated with RNase-free DNase I (Takara, Shiga, Japan) followed by a phenol/chloroform extraction. The concentration of RNA is quantified by spectrophotometric measurement.

Reverse Transcriptase Polymerase Chain Reaction (RT-PCR) Analysis

For RT-PCR, cDNAs synthesized from 2 μg of total leaf RNA using a first-strand cDNA synthesis kit (Invitrogen) are used as the amplification template. Polymerase chain reaction is performed in 50 μl reactions containing 2 μl of 1:20 diluted cDNA samples and 0.2 μm of gene-specific primers using Ex-Taq DNA polymerase (PanVera, Madison, Wis., USA) under the following conditions: an initial denaturation step at 96° C. for 5 min followed by 25 cycles of denaturation at 96° C. for 15 s, annealing at 58° C. for 30 s, polymerization at 72° C. for 1 min, and a final extension at 72° C. for 10 min. Control RT-PCR is performed using a primer pair specific to the ACTIN2 (At3g18780) gene under the same conditions. Twelve microliters of each RT-PCR product is analysed on a 1.0% (w:v) agarose gel to visualize the amplified cDNAs.

For expression analysis of UBA2 genes, senescence activated genes and genes induced by pathogen attack at different developmental stages, PCR is performed using 30 cycles under the same conditions. The primers used in these analyses, 5′ to 3′, are: A1-specific _F: CTATCGCTGCTGCAGCTGTTTCAG, SEQ ID No. 37, UBA2a (At3g56860); A1-UTR_R: GACTTCATTTAGCATCAGCTCCTT, SEQ ID No. 38, UBA2a (At3g56860); A2-specific_F: GGGAACCCTGTTGTGGCTCCTG, SEQ ID No. 39, UBA2b (At2g41060); A2-UTR_R: GGTACCCCATAGATTTTTGTTGG, SEQ ID No. 40, UBA2b (At2g41060); A3-specific_F: CTGGTAAATCTAGAGGCTTTGCAT, SEQ ID No. 41, UBA2c (At3g15010); A3-UTR_R: CAATCAGGTAATCACCACTAAGCA, SEQ ID No. 42, UBA2c (At3g15010); SAG12_F: GACCAATCCAAAAGCAACTTCTAT, SEQ ID No. 43, SAG12 (At5g45890); SAG12 R: TTTAGACATCAATCCCACACAAAC, SEQ ID No. 44, SAG12 (At5g45890); SAG13_F: TGTAATGGCTACAAATCTCGAGTC, SEQ ID No. 45, SAG13 (At2g29350); SAG13_R: CTAGTCTGCCGTCAAATTGGTAAT, SEQ ID No. 46, SAG13 (At2g29350); SAG14_F: AGGACTACGATGTTGGTGATGATA, SEQ ID No. 47, SAG14 (At5g20230); SAG14_R: GAGTGTGACTCAAAAGAGAGCAAC, SEQ ID No. 48, SAG14 (At5g20230); SAG15_F: ACGCATTGATCATAATGACCTCTA, SEQ ID No. 49, SAG15 (At5g51070); SAG15_R: ATACAATCCTCCAATGGTGAAACT, SEQ ID No. 50, SAG15 (At5g51070); SAG101_F: GGTCTCACCACTATGTTATGCTTG, SEQ ID No. 51, SAG101 (At5g14930); SAG101_R: TTACTCTCGCAATGACACACTTTT, SEQ ID No. 52, SAG101 (At5g14930); SIRK_F: ATTTATCTTGAGCTGGGAAGAGAG, SEQ ID No. 53, SIRK (At2g19190); SIRK_R: GGCATACATATTATTCAGCAACCA, SEQ ID No. 54, SIRK (At2g19190); WRKY6_F: AACTGAGTCCAACAAAATTCAGAAG, SEQ ID No. 55, WRKY6 (At1g62300); WRKY6_R: GTGGTTCGTACCATTGATCATAGA, SEQ ID No. 56, WRKY6 (At1g62300); WRKY53_F: GAAGTGACGTACAGAGGAACACAC, SEQ ID No. 57, WRKY53 (At4g23810); WRKY53_R: TGGAAGCTTACAACAAGAAGTCTG, SEQ ID No. 58, WRKY53 (At4g23810); WRKY70_F: CATTTTCTTGGAGGAAATATGGAC, SEQ ID No. 59, WRKY70 (At3g56400); WRKY70_R: GTTTTCCACTCTACATGGCCTAAT, SEQ ID No. 60, WRKY70 (At3g56400); SOD_F: GATGGAAGCTCCTAGAGGAAATCT, SEQ ID No. 61, SOD (At5g18100); SOD_R: CTGGAAATTACGTTCTGGTTTACA, SEQ ID No. 62, SOD (At5g18100); CAB_F: TACAAAGAGTCAGAGCTCATCCAC, SEQ ID No. 63, CAB (At3g54890); CAB_R: GTATTTGGGTTCAAAAGGTTCATC, SEQ ID No. 64, CAB (At3g54890); PR1_F: ATCGTCTTTGTAGCTCTTGTAGGTG, SEQ ID No. 65, PR-1 (At2g14610); PR1_R: TGATACATCCTGCATATGATGCTC, SEQ ID No. 66, PR-1 (At2g14610); PR2_F: CTTACTTCAGCTACATGGGAGACA, SEQ ID No. 67, PR-2 (At3g57260); PR2_R: CAAGTTCCCAATTTTTAAATACGC, SEQ ID No. 68, PR-2 (At3g57260); PR5_F: ATGGCAAATATCTCCAGTATTCACA, SEQ ID No. 69, PR-5 (At1g75030); PR5_R: ATGTCGGGGCAAGCCGCGTTGAGG, SEQ ID No. 70, PR-5 (At1g75030); EDS1_F: AGATGAATACAAGCCAAAGTGTCA, SEQ ID No. 71, EDS1 (AT3G48080); EDS1_R: AGCGTAATCCACCACTTTCTAAAC, SEQ ID No. 72, EDS1 (AT3G48080); ACS2_F: ATTTCATGGGAAAAGCTAGAGGTG, SEQ ID No. 73, ACS2 (At1g01480); ACS2_R: AACTTTATGCCTTATCCCCAACCT, SEQ ID No. 74, ACS2 (At1g01480); ACS6_F: GTAATCGAGGAGATCGAAGATTGTA, SEQ ID No. 75, ACS6 (At4g11280); ACS6_R: TACTCTGCCAACACTTCTTCTTCTT, SEQ ID No. 76, ACS6 (At4g11280); MPK3_F: GCTATAAGAGATGTTGTTCCACCA, SEQ ID No. 77, MPK3 (At3g45640); MPK3_R: GGCAAAGATACTAAGTAGCCATTCG, SEQ ID No. 78, MPK3 (At3g45640); CML38_F: CCAGAAGAGCTTCAAAAGAGTTTC, SEQ ID No. 79, Calmodulin 38 (At1g76650); CML38_R: CAATCGTATTTATTGGGTCACAAA, SEQ ID No. 80, Calmodulin 38 (At1g76650); CK1_F CGTTCTTCTCTGGGAAAGACTTAG, SEQ ID No. 81, Choline Kinasel (At1G71697); CK1_R: AGAAGGGAAGAAACACACAAACTG, SEQ ID No. 82, Choline Kinasel (At1G71697); JR1_F: ACAAGGTGACTCTGGTGTTGTTTA, SEQ ID No. 83, Jamonate-Responsive 1 (At3G16470); JR_R: GGGATATCCAGATTGGTTTCACAT, SEQ ID No. 84, Jamonate-Responsive 1 (At3G16470); WR3_F: ACTTCTCATATGCTCACTGATCCA, SEQ ID No. 85, Wound-Responsive 3 (At5g50200); WR3_R: GAAAGAAGAAGTGTGCAACAAGAC, SEQ ID No. 86, Wound-Responsive 3 (At5g50200); ACT2_F: GACCTTTAACTCTCCCGCTATGTA, SEQ ID No. 87, ACTIN2 (At3g18780); ACT2_R: GCTTTTTAAGCCTTTGATCTTGAG, SEQ ID No. 88, ACTIN2 (At3g18780). Primers are designed using primer3 software (Rozen & Skaletsky, In Krawetz et al., Bioinformatics methods and protocols: methods in molecular biology, Totowa, N.J., Humana Press, pp. 365-386, 2000).

Trypan Blue Staining and Aniline Blue Staining

Cell death is visualized in rosette leaf tissue after staining with lactophenol-Trypan blue according to the method of Adam & Somerville (1996) with a slight modification. Briefly, leaves are immersed in 10 ml of ethanol-lactophenol (2 volumes of ethanol and 1 volume of phenol-glycerol-lactic acid-water (1:1:1:1)) containing 0.05% Trypan blue. Falcon tubes containing the leaves are boiled for 10 min and kept at room temperature for 30 min. The staining solution is then removed and 30 ml of chloral hydrate destaining solution (2.5 g ml⁻¹ of water) is added to the tubes. The leaves are cleared for 2 d with shaking by replacing the destaining solution twice. After destaining, leaves are suspended in 50% glycerol. Leaves are examined under a Zeiss microscope (Axioplan) with white light. Aniline blue staining to detect callose is performed as described by Stone et al. (2000). Briefly, leaves are immersed and vacuum-infiltrated in 10 ml of ethanol-lactophenol (2:1 v:v) and then incubated at 60° C. for 30 min. Leaves are then rinsed in 50% ethanol and stained overnight with aniline blue (0.01% aniline blue powder in 150 mM K₁PO₄, pH 9.5). Before samples are mounted, they are equilibrated in 50% glycerol. Aniline blue staining is visualized using an Olympus BX-60 epifluorescence digital microscope with a DAPI filter (excitation 365 nm, emission 420 nm).

Ethylene Measurement

Five-week-old transgenic Arabidopsis plants (T3 generation) are sprayed once with 15 μM DEX at different times. Three rosette leaves with petioles are excised from each of three different plants at 0, 12, 24, 48, 72 h after DEX treatment. The three excised leaves are incubated for 1.5 h under light in a sealed vial (20 ml) containing 1 ml of water. A headspace sample (1 ml) is drawn from each vial and ethylene levels are analysed using a gas chromatograph (model 5840A; Hewlett Packard, Palo Alto, Calif., USA) equipped with a flame ionization detector and a column of activated alumina.

Forty transgenic Arabidopsis T1 generation lines are examined for each of the three UBA2 genes. More than 50% of the 2×35S-UBA2-OX transgenic lines for each of UBA2a, UBA2b, and UBA2c display severe growth defects consisting of premature cell death and chlorosis. The suffix “OX” used in this example indicates “overexpressing.” These plants eventually died before maturation and seed set, and it was thus not possible to generate constitutive overexpressing transgenic lines under control of the 2×35S promoter. Conversely, in plants that show no deleterious growth phenotype and survive to maturity, no accumulation of FLAG-UBA2s is detectable by immunoblot analysis with anti-FLAG antibody. Consistent with this observation, increased expression levels of the transgenes by RT-PCR analysis of these 2×35SUBA2-OX transgenic plants is undetectable, suggesting suppression of transgene expression.

The steroid hormone DEX inducible pTA7002 vector system described in Aoyama & Chua, Plant J., 11:605-612, 1997) is also used in this example to generate conditional UBA2-OX transgenic Arabidopsis plants. For this, transgenic T1 lines are first screened on half-strength MS media containing hygromycin (25 μg/ml). Detached leaves from hygromycin-resistant plants were treated with 15 μM DEX for 15 h and the tissues assayed by immunoblot analysis with anti-FLAG antibody to identify lines that overexpress UBA2a, UBA2b, or UBA2c. Detached leaves were treated for this screening because DEX treatment of whole plants leads to death of plants overexpressing UBA2 proteins, such that seeds cannot be recovered. The transgenic T2 lines obtained from this primary screening of T1 lines were further analyzed to evaluate whether induction of UBA2 genes by treatment of DEX leads to a cell death-like phenotype, as was the case with the 2×35S-UBA2-OX lines. Expression of UBA2 transgenes after DEX application to transgenic T2 plants was confirmed by western blot analysis with anti-FLAG antibody. Expression of each transgene in DEX-inducible UBA2-OX lines is detectable 6-8 h after treatment with DEX. UBA2-OX plants began to show a visible leaf yellowing/cell death-like phenotype 36-48 h after DEX treatment and the leaf yellowing/cell death-like phenotype was scored in these plants 1 wk after DEX treatment. Such symptoms are observed in all plants expressing each UBA2 transgene upon DEX treatment, suggesting that induction of UBA2 overexpression was the cause of the phenotype. In addition, the same symptoms are observed upon DEX-induced expression of UBA2-YFP fusion proteins indicating that this phenotype is not caused by the epitope tag or by its location within the fusion protein. UBA2c-OX lines show rapid (36 h) and strong development of the phenotype, while a somewhat weaker and slower (48 h) phenotype is observed in UBA2b-OX lines; UBA2a-OX lines in general display an intermediate phenotype. UBA2c-OX lines exhibited more rapid and higher expression levels of the UBA2 transgene compared with UBA2a-OX and UBA2b-OX lines. The appearance of the yellowing phenotype correlates with the expression of the UBA2 transgenes. Plants with a stronger expression of the transgene also exhibit a stronger yellowing phenotype: of the three UBA2 genes, UBA2c-OX transgenic plants shows the highest expression of the encoded UBA2c protein, and the strongest yellowing phenotype.

Further, an independent test of the UBA2 overexpression phenotype, using an estradiol inducible UBA2-OX system, is employed. Use of this system in an agro-infiltration transient assay in Nicotiana benthamiana leaves subsequently treated with 20 μM estradiol confirms that induction of UBA2 transgenes results in yellowing and cell death symptoms. Thus, UBA2 overexpression is responsible for the leaf yellowing/cell death-like phenotype.

Overexpression of UBA2 genes elevates transcript levels of senescence-associated genes and defense-related genes

Leaf yellowing is caused by chlorophyll breakdown and is a characteristic symptom of senescence. The senescence program involves the preferential expression of SAGs. By RT-PCR analysis, the effects of DEX-induced overexpression of UBA2 genes on accumulation of UBA2 and SAG transcripts is examined. Each UBA2-OX line produces an increase in the corresponding UBA2 transcript in rosette leaf tissues from 5-wk-old plants by 24 h after DEX treatment. The expression levels of UBA2c transcripts are slightly higher in UBA2c-OX and UBA2b-OX lines; otherwise, overexpression of any one UBA2 gene does not affect transcript levels of the other UBA2 genes. In parallel with UBA2 overexpression, increased expression of SAGs, which are induced during senescence is observed. SAGs induced during senescence include SAG13, SAG14, SAG15, SAG101, S1RK, WRKY6, WRKY53 and WRKY70, as described Miller et al., Plant Physiol., 120:1015-1024, 1999. An observed exception to this pattern is SAG12. SAG12, which encodes a cysteine protease, has been defined as a senescence-specific gene; it is only expressed in leaves undergoing age-related senescence that are showing signs of chlorosis, see Lohman et al., Physiol. Plantarum, 92:322-328, 1994; and Noh & Amasino, Plant Mol. Biol., 41:181-194, 1999. A slight expression of SAG12 was only detected 48 h after DEX treatment in UBA2b-OX and UBA2c-OX lines. By contrast, some genes, especially those associated with photosynthesis, show downregulation of transcript levels during senescence. The photosynthetic gene encoding chlorophyll-a/b-binding protein (Cab) is one such gene, and it indeed shows a decreased pattern of expression in all three types of UBA2-OX lines at 24 and 48 h, with the greatest decrease observed in UBA2c-OX lines. The transcript level of copper (Cu)/zinc (Zn) SOD is also reduced in the three types of UBA2-OX lines, consistent with the fact that antioxidant enzyme activities generally decrease during senescence. These results imply that expression of UBA2 genes induces premature leaf senescence at least in part through elevating levels of SAGs and decreasing expression of other genes, in a pattern consistent with that observed during the senescence process. As both senescence during plant aging and the hypersensitive response (HR) to avirulent pathogens are forms of programmed cell death, expression patterns of defense-related genes by RT-PCR were also examined. A variety of defense-related genes, including Enhanced Disease Resistance 1 (EDS1), 1-Aminocyclopropane-1-Carboxylic acid Synthase (ACS) genes, and Calmodulin 38 (CML38, At1g76650) are induced upon overexpression of UBA2a, UBA2b, or UBA2c transgenes.

Further, a wound induced and defense-related mitogen-activated protein kinase, MPK3, and wound-responsive genes such as Choline Kinase I (CK1), Jasmonate Responsive 1 (JR1), and Wound Responsive 3 (WR3) are upregulated by overexpression of UBA2 genes. These results suggest that expression of UBA2 genes induces the cell death-like phenotype through elevated levels of a variety of defense- and wounding-related transcripts.

Overexpression of UBA2 proteins results in increased callose deposition that accompanies the cell death phenotype

Results described herein show that constitutive overexpression of UBA2 genes from a 2×35S promoter caused premature plant death that is preceded by severe growth defects. Additionally, DEX-inducible UBA2-OX lines shows a cell death-like phenotype. Thus, to assess cell death in DEX-inducible UBA2-OX lines, 5-wk-old plants from each of two independent lines (T3 generation) are sprayed with 15 μM DEX. Rosette leaves are stained with Trypan blue 48 h after DEX treatment to reveal dead cells and typical regions of dark blue spots indicative of cell death in the rosette leaves from UBA2a-OX, UBA2b-OX, and UBA2c-OX lines are obtained in at least two independent lines for each of the UBA2 transgenes. This indicates that overexpression of UBA2 genes leads to cell death in all three types of UBA2-OX lines. Callose is deposited by numerous cells of plants overexpressing the UBA2 transgenes as observed in at least two independent lines for each UBA2 transgene, whereas such accumulation is not detected in the control lines. This indicates that overexpression of the UBA2 proteins is likely linked to plant defense responses that occur in plant cells undergoing hypersensitive cell death.

UBA2 Transcripts are Not Elevated by Age-Induced Senescence

Assays show that naturally occurring UBA2 genes are not induced in wild-type plants during the natural process of age-related senescence. Wild-type plants are grown under long-day (16 h light:8 h dark cycle) conditions for 10 wk and, starting at 4 wk, leaves are harvested at 1-wk intervals and UBA2 transcript levels are assessed by RT-PCR. No consistent induction pattern of the three UBA2 transcripts by the aging process is found. By contrast, SAG12 showed strong induction starting at 8 wk, i.e. at the same developmental stage where visible leaf yellowing first appears.

Overexpression of UBA2 proteins induces elevated levels of ethylene production

Transcript levels of ACS2 and ACS6, which encode 1-aminocyclopropane-1-carboxylic acid synthase, the key enzyme implicated in ethylene biosynthesis, are enhanced 24 h and 48 h after induction of UBA2 overexpression. In addition, the leaf yellowing symptom of senescence is known to be accelerated by ethylene. Thus, endogenous ethylene levels are measured by gas chromatography in T3 generation plants overexpressing each one of the three UBA2 transgenes. Quantitation of ethylene by gas chromatography shows that overexpression of UBA2a and UBA2b genes gradually increases endogenous levels of ethylene, with a dramatic increase observed by 72 h. For the UBA2c-OX lines, a maximum in ethylene production is observed at 12 h, followed by a gradual decrease, although levels still remains elevated compared with the empty vector control lines. This phenomenon is likely correlated with the rapid appearance of a strong visible leaf yellowing in the UBA2c-OX lines. The increased levels of ethylene caused by overexpression of UBA2 genes upon DEX treatment may be a proximate trigger of accelerated senescence and cell death in the leaves of UBA2-OX transgenic plants.

Nuclear Localization of UBA2-mYFP in Stable Transgenic Arabidopsis Plants

DEX-inducible transgenic Arabidopsis plants expressing UBA2a-, UBA2b-, and UBA2c-mYFP are generated and analysed by confocal microscopy and UBA2-mYFP fusion proteins are exclusively observed in the nuclei of plant cells, including but not limited to guard cells and epidermal cells, after DEX treatment. By contrast, control YFP alone is distributed throughout the cells. This result shows that the UBA2s are nuclear-localized proteins in Arabidopsis. Further, UBA2c-mYFP, but not UBA2a-mYFP or UBA2b-mYFP, fusion protein is localized in nuclear speckles with distinct subnuclear foci.

### Example 2

RNA Extraction

Potato leaves are frozen with liquid nitrogen and ground into powder using a pestle and mortar. The powder is used for RNA extraction using QIAGEN—RNeasy Plant Mini Kit (Qiagen). Total RNAs are reverse-transcribed using SuperScript III (Invitrogen) and used for cloning of potato AKIP cDNAs.

### Example 3

pSAG12:AtAKIP2:Arabidopsis

The pORE-R2 plasmid described in Coutu et al., 2007, Transgenic Res 16: 771-781 is used to generate a vector containing an expression cassette containing a senescence-activated promoter operably linked to an AKIP.

The senescence-activated promoter pSAG12 (SEQ ID No.11) and Arabidopsis AKIP2 (SEQ ID No. 7) are inserted into the pORE-R2 plasmid to generate pORE-R2 harboring the pSAG12:Flag-AKIP2 expression cassette (SEQ ID No. 17).

SEQ ID No. 11 is amplified from Arabidopsis genomic DNA using primers (Forward: 5′-ACACACCGCGGGATATCTCTTTTTATATTCAAACAATAAG-3′ SEQ ID No. 89; and Reverse: 5′-ACACACTCGAGTGTTTTAGGAAAGTTAAATGACTTTTG-3′ SEQ ID No. 90, digested with SacII/XhoI and ligated into SacII/XhoI sites of pORE-R2.

Flag-AKIP2 is excised from pBI121 containing 2×35S:Flag-AKIP2 described in Kim et al., 2008, New Phytol. 180:57-70, using XhoI/SpeI restriction enzymes and ligated into the XhoI/SpeI sites of pORE-R2 harboring the pSAG12 promoter.

The Flag epitope added at the 5′ end of AKIP2 is optional, used for convenience to detect the expressed protein.

This expression cassette is transformed into Agrobacterium strain C58. Overnight culture of Agrobacterium C58 containing pSAG12:Flag-AKIP2 or pSAG12:GUS, SEQ ID No. 18, is transformed into Arabidopsis ecotype Columbia using the method described in Martinez-Trujillo et al., Plant Molecular Biology Reporter 22: 63-70, 2004. Genomic DNA is isolated from transformants following the method described in Edwards et al., Nucl Acid Res, 19:1349, 1991, and used to screen transgenic plants by genomic PCR using primers: Forward: 5′-AATCAGTTGTGTTCATGAGGTG-3′ SEQ ID No. 91; and Reverse: 5′-AGCGGATAACAATTTCACACAGG-3′ SEQ ID No. 92 to produce PCR product SEQ ID No. 20.

As a control for pSAG12:Flag-AKIP2, pORE-R2 with the pSAG12:GUS expression cassette is also transformed into Agrobacterium C58.

GUS expression is examined by GUS staining of leaves from two-month-old pSAG12:GUS transgenic plants and Flag-AKIP2 expression is confirmed by RT-PCR using total RNA from two-month-old Flag-AKIP2 transgenic plants.

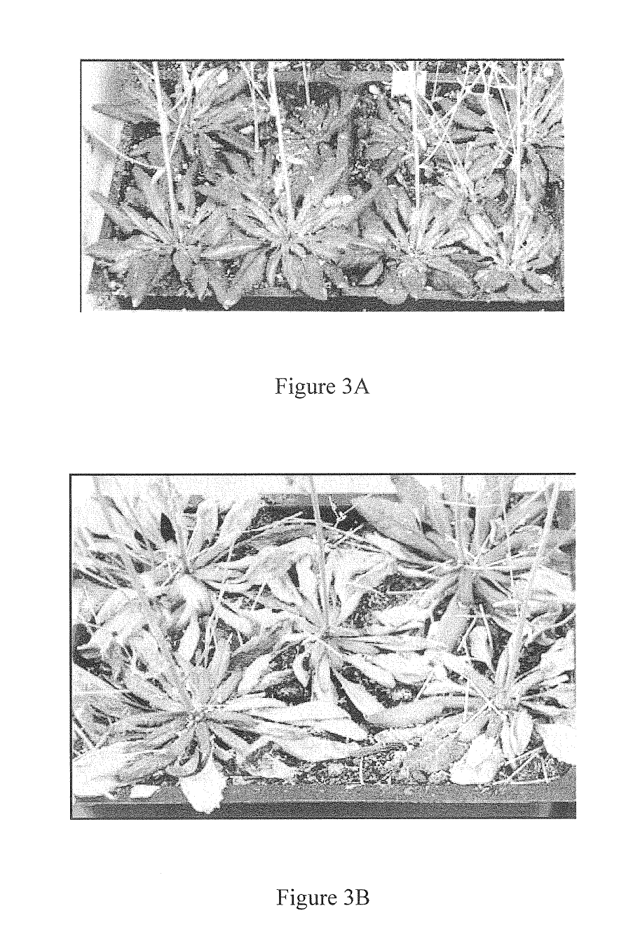

From Arabidopsis transformation using pSAG12:Flag-AKIP2 or pSAG12:GUS expression cassettes, 5 (pSAG12:Flag-AKIP2) and 5 (pSAG12:GUS) T1 lines are confirmed as transgenic by PCR analysis using genomic DNA and grown in a long day growth chamber. In 2 months, 3 pSAG12:Flag-AKIP2 lines exhibit an accelerated senescing phenotype (FIG. 3B), that is not seen in pSAG12:GUS lines (FIG. 3A), indicating that the pSAG12:Flag-AKIP2 expression cassette induces accelerated senescence in plants.

### Example 4

pSAG12:StAKIP1/2:Arabidopsis

The senescence-activated promoter pSAG12 (SEQ ID No.11) and Potato AKIP1/2 cDNA, also called StAKIP1/2, (SEQ ID No. 1) are inserted into the pORE-R2 plasmid to generate pORE-R2 harboring the pSAG12:StAKIP1/2 expression cassette.

SEQ ID No. 11 is amplified from Arabidopsis genomic DNA using primers as described above.

Potato AKIP1/2 cDNA (StAKIP1/2; SEQ ID No. 1) is amplified from potato total cDNA using gene specific primer set: Forward: 5′ CCAAATCAGAAAACCCTAATTTCA-3′ SEQ ID No. 93; and Reverse: 5′-ACTTGGAAGTAGAGCCATAGCATT-3′ SEQ ID No. 94, and cloned into the pORE-R2 plasmid in operable linkage with SEQ ID No.11. This expression cassette is transformed into Agrobacterium strain C58. Overnight culture of Agrobacterium C58 containing pSAG12: StAKIP1/2 or pSAG12:GUS is transformed into Arabidopsis ecotype Columbia. Genomic DNA is isolated from transformants and used to screen transgenic plants by genomic PCR.

### Example 5

pSAG 12:StAKIP3: Arabidopsis

The senescence-activated promoter pSAG12 (SEQ ID No.11) and Potato AKIP3 cDNA, also called StAKIP3 (SEQ ID No. 3) are inserted into the pORE-R2 plasmid to generate pORE-R2 harboring the pSAG12:StAKIP3 expression cassette.

SEQ ID No. 11 is amplified from Arabidopsis genomic DNA using primers as described above. Potato AKIP3 cDNA (StAKIP3; SEQ ID No. 3) is amplified from potato total cDNA using gene specific primer set: Forward: 5′-TCTTCTCTGAGCGAAAATGGA-3′ SEQ ID No. 95; and Reverse: 5′-TCAGGAAATCACCACAACCA-3′ SEQ ID No. 96, and cloned into the pORE-R2 plasmid in operable linkage with SEQ ID No.11. This expression cassette is transformed into Agrobacterium strain C58. Overnight culture of Agrobacterium C58 containing pSAG12:StAKIP3 or pSAG12:GUS is transformed into Arabidopsis ecotype Columbia. Genomic DNA is isolated from transformants and used to screen transgenic plants by genomic PCR.

### Example 6

pSPG31:StAKIP1/2:Arabidopsis

The sweet potato SPG31 gene is homologous to the SAG12 gene and shows very strong up-regulation upon onset of senescence as described in Chen et al., Plant Cell Physiol. 43:984-91, 2002. The SPG31 gene is strongly expressed in leaves, indicating that the SPG31 promoter drives expression that is essentially leaf-specific. Also SPG31 expression is strongly induced by ethylene (C₂H₄), a hormone highly produced during senescence.

The senescence-activated promoter pSPG31 (SEQ ID No.12) and Potato AKIP1/2 (DNA, (SEQ ID No. 1) are inserted into the pORE-R2 plasmid to generate pORE-R2 harboring the pSPG31:StAKIP1/2 expression cassette. This expression cassette is transformed into Agrobacterium strain C58. Overnight culture of Agrobacterium C58 containing pSPG31: StAKIP1/2 or pSAG12:GUS is transformed into Arabidopsis ecotype Columbia. Genomic DNA is isolated from transformants and used to screen transgenic plants by genomic PCR.

### Example 7

pSPG31:StAKIP3:Arabidopsis

The senescence-activated promoter pSPG31 (SEQ ID No.12) and Potato AKIP3 cDNA, (SEQ ID No. 3) are inserted into the _{P}ORE-R2 plasmid to generate pORE-R2 harboring the pSPG31:StAKIP3 expression cassette. This expression cassette is transformed into Agrobacterium strain C58. Overnight culture of Agrobacterium C58 containing pSPG31: StAKIP3 or pSAG12:GUS is transformed into Arabidopsis ecotype Columbia. Genomic DNA is isolated from transformants and used to screen transgenic plants by genomic PCR.

### Example 8

pSAG12:StAKIP1/2:Potato

The senescence-activated promoter pSAG12 (SEQ ID No.11) and Potato AKIP1/2 cDNA, also called StAKIP1/2, (SEQ ID No. 1) are inserted into the pORE-R2 plasmid to generate pORE-R2 harboring the pSAG12:StAKIP1/2 expression cassette.

SEQ ID No. 11 is amplified from Arabidopsis genomic DNA using primers as described above. Potato AKIP1/2 cDNA (StAKIP1/2; SEQ ID No. 1) is amplified from potato total cDNA using gene specific primer set, SEQ ID Nos. 93 and 94, and cloned into the pORE-R2 plasmid in operable linkage with SEQ ID No.11. This expression cassette is transformed into Agrobacterium strain C58. Overnight culture of Agrobacterium C58 containing pSAG12: StAKIP1/2 or pSAG12:GUS is transformed into potato cells for generation of transgenic potato plants.

Potato Plant Materials

Potato plants (cultivar Atlantic) are grown in Magenta tissue culture containers with Murashige and Skoog (MS) medium containing 2% sucrose and solidified with 0.7% agar (Sigma, St. Louis, Mo.). Each magenta box containing potato plants is placed at 20° C. in a growth chamber with 80% relative humidity under 16 hour light of 150 μmol/m²/s intensity from fluorescent lights. Potato plants are subcultured every 2 months. For subculture, shoot tips of 2-month in-vitro grown plants are cut using a sterile scalpel and transferred to new fresh MS media.

Potato Plant Transformation

Leaves from one month in-vitro grown potato plants are cut into explants 0.5-1 cm in length. Explants are cultured in preconditioning MS liquid media containing BAP 10 mg/L and NAA 10 mg/L and 2% sucrose in a rotating incubator with 50 rpm at room temperature for a day. Potato leaf explants are inoculated with overnight culture of Agrobacterium strain C58 containing pORE-R2 plasmid with target constructs, followed by 2-day co-cultivation in callus induction MS media supplemented with IAA 0.018 mg/L and BAP 2.25 mg/L). After a brief wash using sterile H₂O containing 500 mg/L cefotaxim, 2-day co-cultivated explants are transferred and cultured on callus induction MS media containing 50 mg/L kanamycin and 250 mg/L cefotaxim for 7-10 days. Then, explants are transferred and cultured on shoot induction MS media containing Zeatin 2 mg/L and GA₃ 5 mg/L with subculture every 3-4 weeks. Regenerants are transferred to MS media containing 50 mg/L kanamycin and 250 mg/L cefotaxim to induce root formation.

Detection of Transgenic Potato Plants

Genomic DNA is isolated from one-month-old regenerants using the method described in Edwards et al., Nucl Acid Res 19:1349, 1991, and used for screening transformants by genomic PCR using genomic DNA with primers: Forward: 5′-AATCAGTTGTGTTCATGAGGTG-3′ SEQ ID No. 91; and Reverse: 5′-AGCGGATAACAATTTCACACAGG-3′ SEQ ID No. 92, producing PCR product SEQ ID No. 20.

Markers can be used to identify expressed proteins. For instance, GUS expression is confirmed by GUS staining of leaves from two-month-old pSAG12:GUS transgenic plants. Flag-AKIP expression is confirmed by RT-PCR using total RNA from two-month-old Flag-AKIP transgenic plants.

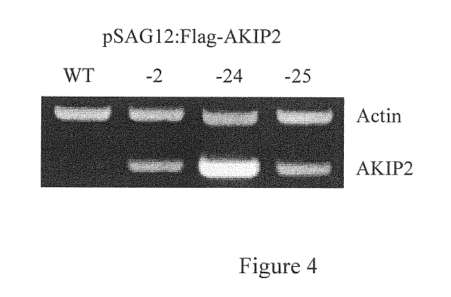

From potato transformation using pSAG12:Fla2-AKIP2 or pSAG12:GUS expression cassettes, each of 50 individual regenerants is obtained. Among them, 24 (pSAG12:Flag-AKIP2) and 15 (pSAG12:GUS) are confirmed as transgenic lines by PCR analysis using genomic DNA isolated from these transgenic plants. AKIP2 expression is detected in 14 pSAG12:Flag-AKIP2 lines including those shown in FIG. 4. pSAG12:GUS lines are subjected to GUS staining and 7 individual lines show GUS expression in 2-month old plants when onset of senescence is imminent/initiated.

### Example 9

pSAG12:StAKIP3:Potato

The senescence-activated promoter pSAG12 (SEQ ID No.11) and Potato AKIP3 cDNA, also called StAKIP3, (SEQ ID No. 3) are inserted into the pORE-R2 plasmid to generate pORE-R2 harboring the pSAG12:StAKIP3 expression cassette.

SEQ ID No. 11 is amplified from Arabidopsis genomic DNA using primers as described above.

Potato AKIP3 cDNA (StAKIP3; SEQ ID No. 3) is amplified from potato total cDNA using gene specific primer set, SEQ ID Nos. 95 and 96, and cloned into the pORE-R2 plasmid in operable linkage with SEQ ID No.11. This expression cassette is transformed into Agrobacterium strain C58. Overnight culture of Agrobacterium C58 containing pSAG12:StAKIP3 is transformed into potato cells for generation of transgenic potato plants.

### Example 10

pSPG31:StAKIP1/2:Potato

The senescence-activated promoter pSPG31 (SEQ ID No.12) and Potato AKIP1/2 cDNA, (SEQ ID No. 1) are inserted into the pORE-R2 plasmid to generate pORE-R2 harboring the pSPG31:StAKIP1/2 expression cassette. This expression cassette is transformed into Agrobacterium strain C58. Overnight culture of Agrobacterium C58 containing pSPG31: StAKIP1/2 is transformed into potato as described in Example 8. Genomic DNA is isolated from transformants and used to screen transgenic plants by genomic PCR.

### Example 11

pSPG31:StAKIP3:Potato

The senescence-activated promoter pSPG31 (SEQ ID No.12) and Potato AKIP3 cDNA, (SEQ ID No. 3) are inserted into the pORE-R2 plasmid to generate pORE-R2 harboring the pSPG31:StAKIP3 expression cassette. This expression cassette is transformed into Agrobacterium strain C58. Overnight culture of Agrobacterium C58 containing pSPG31: StAKIP3 is transformed into potato as described in Example 8. Genomic DNA is isolated from transformants and used to screen transgenic plants by genomic PCR.

### Example 12

Potato AKIP1/2 cDNA (StAKIP1/2; SEQ ID No. 1) is amplified from potato total cDNA using gene specific primer set, SEQ ID Nos. 93 and 94, and cloned into pCR-BluntII Topo plasmid (Invitrogen). BamHI/SpeI fragment containing StAKIP1/2 is then cloned into BamH1/SpeI site of pORE-R2 with 35S:GUS plasmid (SEQ ID No. 14) by replacing the GUS gene to generate pORE-R2 harboring 35S:StAKIP1/2.

### Example 13

Potato AKIP3 cDNA (StAKIP3; SEQ ID No. 3) is amplified from potato total cDNA using gene specific primer set, SEQ ID Nos. 95 and 96, and cloned into pCR-BluntII Topo plasmid (Invitrogen). SacI/SpeI fragment with StAKIP3 is then cloned into SacI/SpeI site of SEQ ID No. 14 by replacing the GUS gene to generate pORE-R2 harboring 35S:StAKIP3.

### Example 14

Expression cassettes, SEQ ID No. 15 and 16, are used for transient expression of potato AKIPs in tobacco plants. Infiltration of Agrobacterium with either expression cassettes SEQ ID No. 15 or 16 is conducted by following the method described in Kim et al., New Phytol. 180:57-70, 2008. Overnight cultures of Agrobacterium C58 with SEQ ID No. 15 or 16 are infiltrated into tobacco leaves and confirmed to induce senescing phenotype.

Two individual tobacco leaves are subjected to transient expression assay. Three Agrobacterium C58 lines are used: one including SEQ ID No. 15, a second including SEQ ID No.16, and a third including 35S:GUS as control are infiltrated (transformed) in separate spots in the same leaf for comparison of signs of senescence, particularly evidence of yellowing and cell death. In both leaves, the spots infiltrated with SEQ ID Nos. 15 and 16 exhibit a strong senescing phenotype in 6 days, and this senescing phenotype is not observed in the control infiltrated with 35S:GUS expression cassette.

### Example 15

pGC1:pSAG12:StAKIP1/2:Potato

The senescence-activated promoter pSAG12 (SEQ ID No.11), the guard-cell specific promoter pGC1 (SEQ ID No. 13) and Potato AKIP1/2 cDNA (SEQ ID No. 1) are inserted into the pORE-R2 plasmid to generate pORE-R2 harboring the pGC1:pSAG12:StAKIP1/2 expression cassette for guard cell specific senescence-activated expression of potato AKIP1/2. This expression cassette is transformed into Agrobacterium strain C58. Overnight culture of Agrobocterium C58 containing pGC1:pSAG12:StAKIP1/2 is transformed into potato cells. Genomic DNA is isolated from transformants and used to screen transgenic plants by genomic PCR.

### Example 16

pSAG2:StAKIP1/2:Potato

SAG2 is a member of a senescence-associated gene family and its expression upon onset of senescence is stronger than SAG12 as shown in Grbic, Physiologia Plantarum 119:263-269. 2003, indicating that the SAG2 promoter is another promoter can be used to induce timely senescence in potato or other plant species.

The senescence-activated promoter pSAG2 (SEQ ID No.19) and Potato AKIP1 cDNA, (SEQ ID No. 1) are inserted into the pORE-R2 plasmid to generate pORE-R2 harboring the pSAG2:StAKIP1/2 expression cassette. This expression cassette is transformed into Agrobacterium strain C58. Overnight culture of Agrobacterium C58 containing pSAG2: StAKIP1/2 is transformed into potato as described in Example 8. Genomic DNA is isolated from transformants and used to screen transgenic plants by genomic PCR.

### Example 17

pSAG2:StAKIP3:Potato

The senescence-activated promoter pSAG2 (SEQ ID No.19) and Potato AKIP3 cDNA. (SEQ ID No. 3) are inserted into the pORE-R2 plasmid to generate pORE-R2 harboring the pSAG2:StAKIP3 expression cassette. This expression cassette is transformed into Agrobacterium strain C58. Overnight culture of Agrobacterium C58 containing pSAG2: StAKIP3 is transformed into potato as described in Example 8. Genomic DNA is isolated from transformants and used to screen transgenic plants by genomic PCR.

### Example 18

pSAG2:StAKIP1/2:Arabidopsis

The senescence-activated promoter pSAG2 (SEQ ID No.19) and Potato AKIP1/2 cDNA, (SEQ ID No. 1) are inserted into the pORE-R2 plasmid to generate pORE-R2 harboring the pSAG2:StAKIP1/2 expression cassette. This expression cassette is transformed into Agrobacterium strain C58. Overnight culture of Agrobacterium C58 containing pSAG2: StAKIP1/2 is transformed into Arabidopsis as described in Example 8. Genomic DNA is isolated from transformants and used to screen transgenic plants by genomic PCR.

### Example 19

pSAG2:StAKIP3:Arabidopsis

The senescence-activated promoter pSAG2 (SEQ ID No.19) and Potato AKIP3 cDNA, (SEQ ID No. 3) are inserted into the pORE-R2 plasmid to generate pORE-R2 harboring the pSAG2:StAKIP3 expression cassette. This expression cassette is transformed into Agrobacterium strain C58. Overnight culture of Agrobacterium C58 containing pSAG2: StAKIP3 is transformed into Arabidopsis as described in Example 1. Genomic DNA is isolated from transformants and used to screen transgenic plants by genomic PCR.

### Example 20

Quantitation of Senescing Phenotype in Transgenic Plants

Quantitative assessment of enhanced senescence phenotype in transgenic plants is performed by various methods such as by measurement of loss of cell viability, chlorophyll content, measurement of ethylene production and/or analysis of expression of senescence-associated genes, compared to a similar wild-type plant.

Cell Viability Test

A sign of senescence in plant cells is cell death. Promotion of senescence in transgenic plants of the present invention can be quantified by comparison of the number of dead cells in a transgenic plant or portion thereof with the number of dead cells in a similar wild-type plant or portion thereof or other appropriate control.

The extent of cell death during the developmental stage of senescence can be examined using vital dyes, such as fluorescein diacetate (FDA), propidium iodide (P1), and trypan blue. For FDA staining, as described in detail in Poffenroth et al., Plant Physiol. 98:1460-1471, 1992, FDA (Sigma-Aldrich, Saint Louis, USA) is used as a 5 μg/ml solution in water. Epidemial tissues are stained for 20 to 30 minutes in the FDA solution before examination under florescence microscope. Following incubation of an epidermal strip derived from a plant with FDA, only live cells exhibit green fluorescence when examined using a fluorescence microscope equipped with an appropriate light source and bandpass filters.

Leaf Yellowing and Measurement of Chlorophyll Content

A sign of senescence in plant cells is leaf yellowing. Promotion of senescence in transgenic plants of the present invention can be quantified by comparison of the number of yellow leaves and/or extent of leaf yellowing in a transgenic plant or portion thereof with the same parameter in a similar wild-type plant or portion thereof or other appropriate control.

Leaf yellowing is caused by chlorophyll breakdown and is a characteristic symptom of senescence (Miller et al., Plant Physiol 120:1015-1024, 1999). Therefore, chlorophyll content is a good indicator of the degree of senescence. Promotion of senescence in transgenic plants of the present invention can be quantified by comparison of the amount of chlorophyll in a transgenic plant or portion thereof with the amount of chlorophyll in a similar wild-type plant or portion thereof or other appropriate control. To measure chlorophyll content, chlorophyll is extracted using dimethyl formamide (DMF) from freeze-dried leaf tissues and chlorophyll-containing DMF solutions are analyzed by their absorbance at 646.8 and 663.8 nm using a spectrophotometer. Chlorophyll a+b concentration in nmol mL⁻¹ is calculated as 19.43^{646.8}+8.05 A^{663.8} after subtraction of absorbance at 750 nm, following the formula of Porra et al. 1989. Biochem et Biophys Acta 975: 384-394.

Ethylene Measurement

Ethylene production is another indicator of the degree of senescence. In Arabidopsis transgenic plants, it is noted that transcript levels of ACS2 and ACS6, which encode 1-aminocyclopropane-1-carboxylic acid synthase, the key enzyme implicates in ethylene biosynthesis, were shown to be enhanced 24 h and 48 h after induction of UBA2 overexpression (Kim et al., 2008 New Phytol. 180:57-70.

Promotion of senescence in transgenic plants of the present invention can be quantified by comparison of the amount of ethylene produced in a transgenic plant or portion thereof with the amount of ethylene produced in a similar wild-type plant or portion thereof or other appropriate control. Transgenic plants are used for ethylene measurement following the method described by Kim et al., 2008 New Phytol. 180:57-70. Three leaves with petioles are excised and incubated for 1.5 h under light in a sealed vial (20 ml) containing 1 ml of water. A headspace sample (1 ml) is drawn from each vial and ethylene levels are analysed using a gas chromatograph (model 5840A; Hewlett Packard, Palo Alto, Calif., USA). Transgenic Arabidopsis plants overexpressing AKIPs were shown to have significantly higher ethylene production compared to control plants (Kim et al., 2008 New Phytol. 180:57-70).

RT-PCR Analysis of Senescence Associated Genes (SAGs)

The senescence program involves the preferential expression of SAGs, indicating that SAG expression can be used to quantify the degree of senescence. Promotion of senescence in transgenic plants of the present invention can be quantified by comparison of SAG expression in a transgenic plant or portion thereof with SAG expression in a similar wild-type plant or portion thereof or other appropriate control. RT-PCR analyses are used to quantify SAG expression. In Arabidopsis, in parallel with UBA2-induced overexpression, increased expression of SAGs was observed, including SAG13, SAG14, SAG15, SAG101 (Kim et al., 2008 New Phytol. 180:57-70).

### Example 21

Identification of potato AKIP homologs substantially similar to Arabididopsis AKIPs

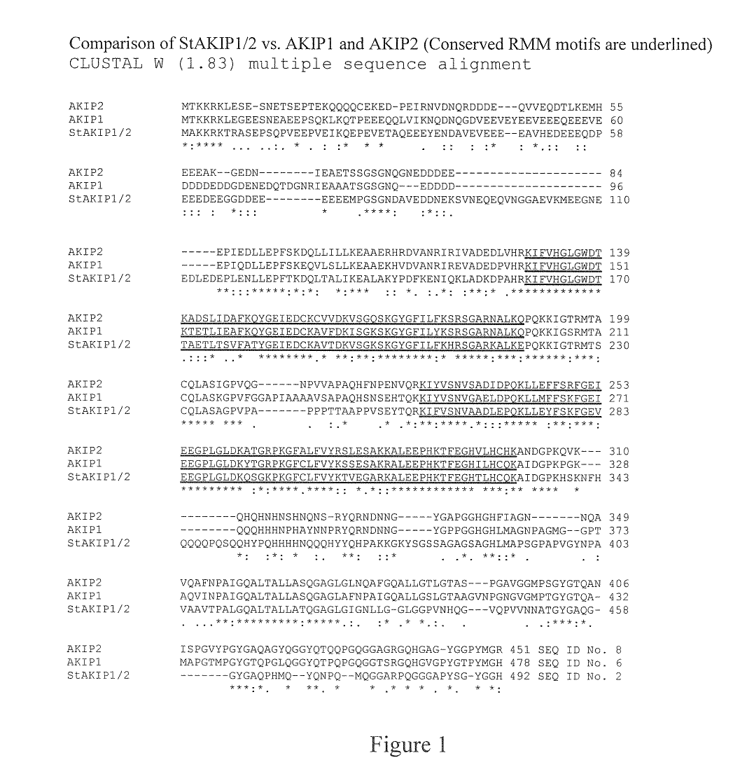

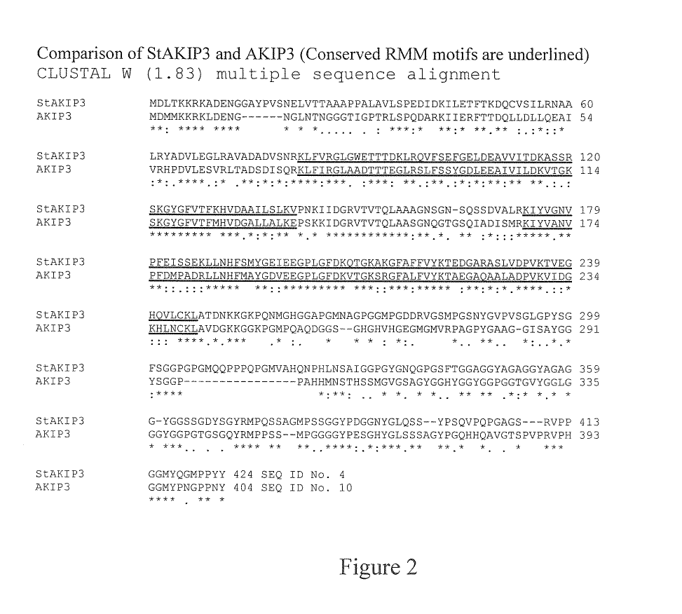

Arabidopsis AKIP protein sequences (SEQ ID No. 6, 8, and 10) are used for blast search to identify potato AKIP-like cDNAs from potato EST database (TIGR Plant Transcript Assemblies Database). Two potato AKIP-like ESTs are found from blast search and re-aligned to Arabidopsis AKIP protein sequences to examine whether RNA binding motifs (RRM) of StAKIPs, which are functional domains of RNA binding proteins, are conserved. CLUSTAL-W alignment shows functional domains of StAKIPs are well conserved and highly similar to RRMs of Arabidopsis AKIPs, indicating that newly identified StAKIPs are potato homologs of Arabidopsis AKIPs. RRM motifs of AKIPs and StAKIPs and their identities are shown in FIGS. 1 and 2 and in Table 1. To clone potato StAKIP cDNAs, EST sequences are used for primer design for StAKIP1/2 (SEQ ID No. 1) or StAKIP3 (SEQ ID No. 2) as described in Example 9 and 10. Because conserved motifs of AKIPs are well conserved not only in protein sequences but also in cDNA sequences, cDNA sequences corresponding to conserved RRM motifs in AKIPs can be used for designing degenerate primers. This method is can be used to amplify and identify potential AKIP homologs by PCR amplification method from the cDNA of any plant species. Newly identified AKIP-like genes are tested by transient expression assay to detect senescing phenotype similar to At AKIPs and StAKIPs, as a functional test of AKIP identity.

Sequences

| StAKIP1/2, 1577 bases | |
|---|---|
| SEQ ID No. 1 | |
| 1 | CCAAATCAGA AAACCCTAAT TTCAACTATT TCTCTCTCTA TACCTCAATA TTCCCATGGC |
| 61 | GAAGAAGCGA AAAACCAGAG CTTCCGAACC TTCACAACCA GTTGAAGAAC CAGTAGAGAT |
| 121 | TAAGCAAGAA CCTGAGGTTG AAACCGCACA AGAAGAAGAA TACGAGAACG ATGCCGTAGA |
| 181 | AGTAGAGGAA GAAGAAGCAG TACATGAGGA CGAAGAAGAA CAAGATCCCG AAGAGGAAGA |
| 241 | CGAAGAAGGC GGTGACGATG AGGAGGAAGA AGAAGAAATG CCCGGATCTG GAAATGACGC |
| 301 | TGTAGAGGAT GATAACGAGA AGTCGGTTAA TGAACAGGAA CAAGTTAATG GAGGCCCTGA |
| 361 | GGTGAAGATG GAGGAAGGTA ATGAAGAGGA TTTGGAAGAT GAGCCACTTG AGAATCTGTT |
| 421 | AGAGCCATTT ACGAAGGATC AACTTACAGC GCTTATTAAA GAAGCTTTAG CTAAATACCC |
| 481 | TGATTTCAAA GAAAATATTC AGAAATTGGC TGATAAAGAT CCGGCACACC GGAAAATCTT |
| 541 | TGTCCACGGT TTAGGTTGGG ATACGACAGC TGAAACGCTA ACTAGTGTTT TTGCAACTTA |
| 601 | CGGTGAAATT GAGGATTGTA AAGCTGTTAC AGATAAGGTT TCGGGGAAAT CGAAAGGTTA |
| 661 | TGGATTTATA TTGTTTAAGC ATAGGAGTGG TGCTAGGAAA GCTTTGAAAG AACCACAGAA |
| 721 | GAAGATTGGT ACAAGGATGA CTTCTTGCCA GTTGGCTTCT GCTGGGCCGG TTCCGGCTCC |
| 781 | TCCACCTACT ACAGCAGCCC CACCGGTTTC GGAGTACACA CAGAGGAAAA TTTTTGTTAG |
| 841 | CAATGTAGCT GCTGATCTTG AACCTCAAAA GTTGTTGGAG TACTTCTCAA AGTTTGGTGA |
| 901 | AGTTGAGGAG GGTCCACTTG GATTGGATAA GCAAAATGGG AAACCTAAGG GGTTTTGTTT |
| 961 | GTTTGTGTAT AAGACTGTTG AGGGTGCTAG GAAGGCATTG GAGGAGCCAC ATAAGACCTT |
| 1021 | TGAAGGGCAT ACCCTTCATT GTCAGAAGGC GATTGATGGA CCGAAGCATA GTAAGAATTT |
| 1081 | CCATCAGCAG CAACAGCCTC AGTCACAACA GCATTACCCT CAGCATCATC ATCATAATCA |
| 1141 | GCAGCAGCAT TATTATCAGC ATCCTGCAAA GAAGGGGAAA TATTCAGGTA GCAGTGCAGG |
| 1201 | AGCAGGATCA GCTGGGCATT TAATGGCACC ATCGGGGCCT GCACCTGTGG GATACAATCC |
| 1261 | TGCTGTGGCA GCTGTGACCC CAGCTCTTGG ACAGGCACTG ACAGCCCTGT TAGCTACACA |
| 1321 | AGGAGCTGGT TTGGGGATTG GGAATTTGCT TGGAGGTTTT GGTGGGCCAG TGAATCATCA |
| 1381 | GGGTGTGCAA CCAGTGGTGA ACAATGCAAC AGGATATGGT GCCCAGGGTG GATATGGAGC |
| 1441 | TCAGCCTCAT ATGCAATACC AAAACCCTCA GATGCAGGGT GGTGCAAGAC CACAAGGTGG |
| 1501 | TGGTGCCCCA TACTCTGGCT ATGGAGGTCA CTAGGAAAAC TTAAGGTATC TGTAATGCTA |
| 1561 | TGGCTCTACT TCCAAGT |
| StAKIP1/2, 492 aa | |
| SEQ ID No. 2 | |
| 1 | MAKKRKTRAS EPSQPVEEPV EIKQEPEVET AQEEEYENDA VEVEEEEAVH EDEEEQDPEE |
| 61 | EDEEGGDDEE EEEEMPGSGN DAVEDDNEKS VNEQEQVNGG PEVKMEEGNE EDLEDEPLEN |
| 121 | LLEPFTKDQL TALIKEALAK YPDFKENIQK LADKDPAHRK IFVHGLGWDT TAETLTSVFA |
| 181 | TYGEIEDCKA VTDKVSGKSK GYGFILFKHR SGARKALKEP QKKIGTRMTS CQLASAGPVP |
| 241 | APPPTTAAPP VSEYTQRKIF VSNVAADLEP QKLLEYFSKF GEVEEGPLGL DKQNGKPKGF |
| 301 | CLFVYKTVEG ARKALEEPHK TFEGHTLHCQ KAIDGPKHSK NFHQQQQPQS QQHYPQHHHH |
| 361 | NQQQHYYQHP AKKGKYSGSS AGAGSAGHLM APSGPAPVGY NPAVAAVTPA LGQALTALLA |
| 421 | TQGAGLGIGN LLGGFGGPVN HQGVQPVVNN ATGYGAQGGY GAQPHMQYQN PQMQGGARPQ |
| 481 | GGGAPYSGYG GH |
| StAKIP3, 1349 bases | |
| SEQ ID No. 3 | |
| 1 | TCTTCTCTGA GCGAAAATGG ATCTAACAAA GAAACGCAAA GCTGATGAGA ACGGCGGTGC |
| 61 | TTATCCCGTA TCCAACGAAC TCGTCACAAC TGCCGCCGCG CCGCCGGCAC TTGCTGTTCT |
| 121 | TTCCCCGGAA GACATCGACA AAATTCTTGA AACATTCACC AAAGACCAGT GCGTTTCAAT |
| 181 | TCTCCGTAAC GCCGCCCTAC GCTATGCTGA TGTTCTCGAA GGTCTACGTG CTGTCGCTGA |
| 241 | CGCCGACGTA TCTAACCGCA AGCTCTTCGT TCGTGGTCTT GGTTGGGAGA CCACCACCGA |
| 301 | CAAACTCCGA CAGGTTTTTT CTGAGTTTGG AGAACTCGAT GAGGCTGTAG TTATAACTGA |
| 361 | TAAGGCGTCT TCCAGATCTA AAGGGTATGG TTTTGTAACT TTCAAGCACG TTGATGCTGC |
| 421 | TATTCTTTCT TTAAAGGTGC CCAACAAGAT AATTGATGGC CGTGTTACCG TCACACAGCT |
| 481 | TGCTGCTGCG GGTAATTCTG GGAATTCTCA GTCTTCTGAT GTTGCGCTTA GGAAGATCTA |
| 541 | TGTTGGTAAT GTTCCATTTG AAATTTCGTC TGAGAAGCTT TTGAATCACT TTTCTATGTA |
| 601 | TGGAGAGATT GAAGAGGGGC CTTTGGGATT TGATAAACAA ACTGGAAAAG CTAAAGGTTT |
| 661 | TGCTTTTTTT GTCTACAAGA CTGAGGACGG AGCTAGGGCA TCTTTAGTTG ATCCCGTGAA |
| 721 | GACAGTAGAA GGACATCAGG TCTTGTGTAA ATTGGCAACC GATAATAAGA AGGGGAAGCC |
| 781 | CCAGAATATG GGGCATGGGG GTGCCCCTGG AATGAATGCT GGACCTGGAG GAATGCCTGG |
| 841 | TGATGATAGG GTTGGATCCA TGCCCGGTTC AAATTATGGT GTTCCTGTTA GCGGTTTGGG |
| 901 | TCCATATTCA GGGTTTTCAG GTGGGCCTGG TCCAGGAATG CAGCAGCCAC CACCTCAGCC |
| 961 | TGGCATGGTG GCACATCAGA ATCCACATCT GAATTCAGCT ATTGGCGGGC CTGGATATGG |
| 1021 | AAACCAAGGA CCAGGATCCT TTACAGGTGG TGCTGGTGGA TATGCTGGCG CTGGTGGATA |
| 1081 | TGCTGGGGCT GGTGGGTATG GTGGTAGTTC TGGGGATTAC AGTGGGTACA GGATGCCTCA |
| 1141 | AAGCTCTGCT GGAATGCCTA GTTCAGGAGG TTATCCAGAT GGTGGTAATT ATGGTTTGCA |
| 1201 | GTCTTCTTAT CCCTCACAGG TACCACAACC AGGAGCTGGG TCAAGGGTTC CACCAGGTGG |
| 1261 | GATGTACCAG GGCATGCCTC CATACTACTG AGGTGTCTTG AGAGCTTCTT GGATGCAACA |
| 1321 | TGCTTTAAAT GGTTGTGGTG ATTTCCTGA |
| StAKIP3, 424 aa | |
| SEQ ID No. 4 | |
| 1 | MDLTKKRKAD ENGGAYPVSN ELVTTAAAPP ALAVLSPEDI DKILETFTKD QCVSILRNAA |
| 61 | LRYADVLEGL RAVADADVSN RKLFVRGLGW ETTTDKLRQV FSEFGELDEA VVITDKASSR |
| 121 | SKGYGFVTFK HVDAAILSLK VPNKIIDGRV TVTQLAAAGN SGNSQSSDVA LRKIYVGNVP |
| 181 | FEISSEKLLN HFSMYGEIEE GPLGFDKQTG KAKGFAFFVY KTEDGARASL VDPVKTVEGH |
| 241 | QVLCKLATDN KKGKPQNMGH GGAPGMNAGP GGMPGDDRVG SMPGSNYGVP VSGLGPYSGF |
| 301 | SGGPGPGMQQ PPPQPGMVAH QNPHLNSAIG GPGYGNQGPG SFTGGAGGYA GAGGYAGAGG |
| 361 | YGGSSGDYSG YRMPQSSAGM PSSGGYPDGG NYGLQSSYPS QVPQPGAGSR VPPGGMYQGM |
| 421 | PPYY |
| AKAIP1, 1437 bp (AT3G56860) | |
| SEQ ID No. 5 | |
| 1 | ATGACAAAGA AGAGAAAGCT CGAAGGAGAA GAATCTAACG AAGCTGAAGA ACCGTCGCAG |
| 61 | AAGCTGAAGC AGACGCCGGA GGAGGAGCAG CAGCTTGTAA TTAAAAATCA GGATAATCAA |
| 121 | GGAGACGTTG AAGAAGTTGA ATACGAGGAA GTAGAGGAAG AGCAAGAAGA AGAAGTAGAA |
| 181 | GATGATGACG ATGAAGATGA TGGTGATGAG AATGAGGATC AGACGGATGG GAATCGTATA |
| 241 | GAGGCGGCGG CGACGTCTGG ATCTGGCAAT CAAGAAGATG ATGACGATGA ACCAATTCAG |
| 301 | GATCTGTTGG AACCATTTTC AAAGGAGCAA GTTTTGAGTC TTCTTAAGGA AGCAGCGGAG |
| 361 | AAGCATGTTG ATGTAGCCAA TCGAATTAGG GAAGTAGCTG ATGAGGACCC TGTTCATCGG |
| 421 | AAGATCTTTG TGCACGGTCT TGGTTGGGAT ACGAAAACAG AGACGCTTAT TGAAGCTTTT |
| 481 | AAGCAGTACG GAGAGATCGA AGATTGCAAG GCTGTGTTTG ATAAGATCTC AGGGAAATCT |
| 541 | AAAGGTTATG GCTTTATTCT GTACAAGTCT CGTTCAGGTG CTCGCAACGC ACTCAAGCAG |
| 601 | CCTCAGAAGA AGATTGGTAG TCGTATGACG GCATGCCAAT TGGCTTCTAA AGGACCTGTC |
| 661 | TTTGGTGGAG CTCCTATCGC TGCTGCAGCT GTTTCAGCGC CTGCTCAGCA TTCGAACTCG |
| 721 | GAGCATACTC AGAAAAAGAT TTATGTTAGT AATGTCGGAG CAGAGCTTGA TCCACAGAAG |
| 781 | TTGCTGATGT TTTTCTCAAA GTTTGGAGAA ATTGAAGAAG GTCCTTTGGG TCTTGATAAG |
| 841 | TATACTGGGA GACCTAAAGG TTTCTGTCTC TTTGTTTATA AGTCGTCTGA AAGCGCCAAG |
| 901 | AGGGCTTTGG AGGAGCCACA CAAGACTTTT GAAGGCCATA TCTTGCATTG CCAGAAAGCT |
| 961 | ATCGATGGTC CCAAACCGGG CAAGCAGCAG CAGCATCACC ATAACCCACA CGCCTATAAC |
| 1021 | AATCCTCGTT ACCAAAGGAA TGATAACAAT GGTTATGGTC CGCCTGGAGG TCATGGACAT |
| 1081 | CTCATGGCTG GTAACCCAGC TGGGATGGGT GGTCCAACGG CACAGGTGAT AAACCCGGCC |
| 1141 | ATTGGACAGG CCTTGACAGC TTTATTGGCA TCTCAGGGAG CTGGTTTGGC TTTTAATCCG |
| 1201 | GCAATTGGAC AGGCTTTGTT GGGTTCCTTG GGGACAGCTG CTGGTGTAAA CCCAGGGAAT |
| 1261 | GGAGTTGGAA TGCCAACTGG TTACGGTACT CAAGCTATGG CACCGGGGAC AATGCCTGGG |
| 1321 | TACGGTACAC AACCTGGGTT GCAAGGCGGT TATCAGACTC CGCAACCTGG TCAAGGCGGT |
| 1381 | ACAAGTAGAG GGCAACATGG TGTCGGGCCA TACGGTACTC CTTACATGGG TCACTAA |
| AKIP1, 478 aa | |
| SEQ ID No. 6 | |
| 1 | MTKKRKLEGE ESNEAEEPSQ KLKQTPEEEQ QLVIKNQDNQ GDVEEVEYEE VEEEQEEEVE |
| 61 | DDDDEDDGDE NEDQTDGNRI EAAATSGSGN QEDDDDEPIQ DLLEPFSKEQ VLSLLKEAAE |
| 121 | KHVDVANRIR EVADEDPVHR KIFVHGLGWD TKTETLIEAF KQYGEIEDCK AVFDKISGKS |
| 181 | KGYGFILYKS RSGARNALKQ PQKKIGSRMT ACQLASKGPV FGGAPIAAAA VSAPAQHSNS |
| 241 | EHTQKKIYVS NVGAELDPQK LLMFFSKFGE IEEGPLGLDK YTGRPKGFCL FVYKSSESAK |
| 301 | RALEEPHKTF EGHILHCQKA IDGPKPGKQQ QHHHNPHAYN NPRYQRNDNN GYGPPGGHGH |
| 361 | LMAGNPAGMG GPTAQVINPA IGQALTALLA SQGAGLAFNP AIGQALLGSL GTAAGVNPGN |
| 421 | GVGMPTGYGT QAMAPGTMPG YGTQPGLQGG YQTPQPGQGG TSRGQHGVGP YGTPYMGH |
| Arabidopsis AKIP2 (AtAKIP2), 1356 bases AT2G41060 | |
| SEQ ID No. 7 | |
| 1 | ATGACAAAGA AGAGAAAGCT CGAATCTGAA TCCAACGAAA CGTCAGAGCC GACGGAGAAG |
| 61 | CAGCAGCAGC AATGTGAAAA AGAGGATCCG GAAATCAGAA ATGTTGATAA TCAAAGAGAC |
| 121 | GACGACGAAC AAGTAGTAGA GCAAGACACA CTAAAGGAGA TGCACGAAGA GGAAGCTAAA |
| 181 | GGTGAAGATA ACATAGAAGC GGAGACGTCG TCCGGATCTG GGAATCAAGG AAATGAGGAT |
| 241 | GATGACGAAG AAGAACCTAT TGAGGATCTA TTGGAACCGT TTTCAAAGGA TCAACTTTTG |
| 301 | ATTCTTCTCA AGGAAGCTGC AGAGAGACAT CGCGATGTAG CTAATCGAAT CCGGATTGTG |
| 361 | GCGGATGAAG ATCTTGTTCA TCGTAAGATC TTCGTTCACG GGCTTGGATG GGATACTAAA |
| 421 | GCTGATTCAC TTATCGACGC TTTTAAACAG TACGGAGAGA TTGAAGATTG CAAATGTGTG |
| 481 | GTTGATAAGG TATCTGGGCA ATCTAAAGGT TATGGCTTTA TCCTCTTTAA GTCAAGGTCT |
| 541 | GGTGCTCGTA ACGCTCTTAA GCAGCCTCAG AAGAAGATTG GAACTCGTAT GACTGCGTGC |
| 601 | CAGCTGGCGT CTATAGGACC TGTTCAGGGG AACCCTGTTG TGGCTCCTGC TCAGCATTTC |
| 661 | AATCCTGAGA ATGTTCAGAG GAAGATTTAT GTCAGTAACG TTAGTGCAGA CATTGATCCG |
| 721 | CAGAAGTTGC TGGAGTTTTT CTCAAGGTTT GGGGAGATAG AAGAAGGTCC TTTGGGGCTT |
| 781 | GATAAAGCTA CTGGGAGACC TAAAGGTTTC GCTTTGTTTG TCTATAGATC CCTAGAAAGT |
| 841 | GCCAAGAAGG CATTGGAGGA GCCACACAAG ACTTTTGAAG GCCATGTCTT GCATTGCCAC |
| 901 | AAAGCAAATG ATGGGCCAAA ACAGGTTAAG CAACATCAAC ATAACCATAA CTCTCACAAT |
| 961 | CAAAATTCCC GTTACCAAAG GAACGACAAC AATGGTTATG GTGCCCCTGG AGGCCATGGA |
| 1021 | CATTTCATAG CTGGTAATAA CCAAGCTGTG CAGGCGTTTA ATCCGGCCAT TGGCCAGGCC |
| 1081 | CTCACAGCTT TGCTGGCATC TCAGGGTGCT GGGTTGGGTT TAAACCAAGC ATTTGGGCAG |
| 1141 | GCTTTGTTGG GGACATTAGG GACAGCTAGC CCAGGAGCTG TAGGTGGAAT GCCAAGTGGC |
| 1201 | TATGGTACTC AAGCAAATAT CTCACCTGGG GTCTATCCTG GGTACGGTGC TCAAGCCGGG |
| 1261 | TACCAGGGCG GTTATCAGAC TCAGCAACCT GGTCAGGGCG GTGCGGGAAG AGGGCAGCAT |
| 1321 | GGTGCTGGGT ATGGTGGTCC TTACATGGGT CGTTAG |
| AKIP2, 451 aa | |
| SEQ ID No. 8 | |
| 1 | MTKKRKLESE SNETSEPTEK QQQQCEKEDP EIRNVDNQRD DDEQVVEQDT LKEMHEEEAK |
| 61 | GEDNIEAETS SGSGNQGNED DDEEEPIEDL LEPFSKDQLL ILLKEAAERH RDVANRIRIV |
| 121 | ADEDLVHRKI FVHGLGWDTK ADSLIDAFKQ YGEIEDCKCV VDKVSGQSKG YGFILFKSRS |
| 181 | GARNALKQPQ KKIGTRMTAC QLASIGPVQG NPVVAPAQHF NPENVQRKIY VSNVSADIDP |
| 241 | QKLLEFFSRF GEIEEGPLGL DKATGRPKGF ALFVYRSLES AKKALEEPHK TFEGHVLHCH |
| 301 | KANDGPKQVK QHQHNHNSHN QNSRYQRNDN NGYGAPGGHG HFIAGNNQAV QAFNPAIGQA |
| 361 | LTALLASQGA GLGLNQAFGQ ALLGTLGTAS PGAVGGMPSG YGTQANISPG VYPGYGAQAG |
| 421 | YQGGYQTQQP GQGGAGRGQH GAGYGGPYMG R |
| AKIP3, 1215 bp (At3g15010) | |
| SEQ ID No. 9 | |
| 1 | ATGGATATGA TGAAGAAGCG TAAGCTCGAT GAGAACGGTA ACGGACTCAA TACCAACGGT |
| 61 | GGCGGAACCA TTGGTCCAAC TCGATTGTCG CCGCAAGACG CGAGGAAGAT TATCGAGCGA |
| 121 | TTCACCACTG ATCAGCTGCT CGACTTATTG CAAGAGGCTA TCGTACGCCA TCCCGACGTG |
| 181 | CTTGAATCTG TCCGATTAAC CGCCGACTCT GATATCTCGC AGCGGAAGCT ATTCATCCGT |
| 241 | GGTCTCGCGG CGGATACTAC TACGGAAGGT CTCCGTTCGC TTTTTTCCAG TTACGGAGAT |
| 301 | CTAGAAGAAG CTATTGTGAT CCTTGACAAG GTCACTGGGA AATCGAAAGG GTATGGTTTC |
| 361 | GTCACTTTTA TGCACGTTGA TGGAGCTTTA CTAGCTCTGA AAGAACCATC TAAGAAAATT |
| 421 | GATGGGCGTG TTACAGTAAC GCAGCTCGCG GCATCTGGAA ATCAAGGTAC GGGTTCCCAG |
| 481 | ATTGCAGATA TATCAATGAG GAAGATCTAT GTGGCGAATG TTCCTTTTGA TATGCCGGCG |
| 541 | GATAGGCTAT TGAATCACTT TATGGCTTAT GGAGATGTTG AGGAAGGTCC TTTGGGTTTT |
| 601 | GATAAGGTTA CTGGTAAATC TAGAGGCTTT GCATTGTTTG TCTACAAGAC AGCGGAAGGT |
| 661 | GCGCAGGCTG CTCTTGCTGA TCCGGTGAAG GTGATTGATG GAAAACATTT GAATTGTAAG |
| 721 | TTGGCTGTTG ATGGTAAGAA AGGAGGGAAG CCTGGAATGC CACAAGCTCA AGATGGTGGT |
| 781 | TCTGGTCATG GACACGTTCA TGGTGAGGGA ATGGGAATGG TGCGTCCTGC AGGACCTTAT |
| 841 | GGTGCAGCTG GTGGTATCAG TGCTTATGGT GGGTACTCTG GAGGACCACC AGCGCATCAC |
| 901 | ATGAATTCCA CACACTCCTC AATGGGTGTT GGTTCTGCTG GGTATGGTGG ACACTATGGT |
| 961 | GGATATGGAG GTCCAGGTGG TACTGGCGTC TACGGTGGAC TTGGCGGTGG GTATGGTGGG |
| 1021 | CCTGGTACTG GAAGTGGGCA GTATAGAATG CCACCAAGTT CGATGCCTGG TGGTGGTGGT |
| 1081 | TATCCCGAGA GTGGGCATTA CGGTCTTTCA TCATCTGCTG GGTATCCTGG ACAGCATCAT |
| 1141 | CAGGCTGTTG GAACGTCCCC TGTGCCAAGA GTTCCTCATG GCGGAATGTA CCCCAACGGT |
| 1201 | CCACCAAACT ACTGA |
| AKIP3, 404 aa | |
| SEQ ID No. 10 | |
| 1 | MDMMKKRKLD ENGNGLNTNG GGTIGPTRLS PQDARKIIER FTTDQLLDLL QEAIVRHPDV |
| 61 | LESVRLTADS DISQRKLFIR GLAADTTTEG LRSLFSSYGD LEEAIVILDK VTGKSKGYGF |
| 121 | VTFMHVDGAL LALKEPSKKI DGRVTVTQLA ASGNQGTGSQ IADISMRKIY VANVPFDMPA |
| 181 | DRLLNHFMAY GDVEEGPLGF DKVTGKSRGF ALFVYKTAEG AQAALADPVK VIDGKHLNCK |
| 241 | LAVDGKKGGK PGMPQAQDGG SGHGHVHGEG MGMVRPAGPY GAAGGISAYG GYSGGPPAHH |
| 301 | MNSTHSSMGV GSAGYGGHYG GYGGPGGTGV YGGLGGGYGG PGTGSGQYRM PPSSMPGGGG |
| 361 | YPESGHYGLS SSAGYPGQHH QAVGTSPVPR VPHGGMYPNG PPNY |
| pSAG12 promoter, 2188 bases | |
| SEQ ID No. 11 | |
| 1 | gatatctctt tttatattca aacaataagt tgagatatgt ttgagaagag gacaactatt |
| 61 | ctcgtggagc accgagtttg ttttatatta gaaacccgat tgttattttt agactgagac |
| 121 | aaaaaagtaa aatcgttgat tgttaaaatt taaaattagt ttcattacgt ttcgataaaa |
| 181 | aaatgattag tttatcatag cttaattata gcattgattt ctaaatttgt tttttgacca |
| 241 | cccttttttc tctctttggt gttttcttaa cattagaaga acccataaca atgtacgttc |
| 301 | aaattaatta aaaacaatat ttccaagttt tatatacgaa acttgttttt tttaatgaaa |
| 361 | acagttgaat agttgattat gaattagtta gatcaatact caatatatga tcaatgatgt |
| 421 | atatatatga actcagttgt tatacaagaa atgaaaatgc tatttaaata ccgatcatga |
| 481 | agtgttaaaa agtgtcagaa tatgacatga agcgttttgt cctaccgggt attcgagtta |
| 541 | taggtttgga tctctcaaga atattttggg ccatattagt tatatttggg cttaagcgtt |
| 601 | ttgcaaagag acgaggaaga aagattgggt caagttaaca aaacagagac actcgtatta |
| 661 | gttggtactt tggtagcaag tcgatttatt tgccagtaaa aacttggtac acaactgaca |
| 721 | actcgtatcg ttattagttt gtacttggta cctttggttc aagaaaaagt tgatatagtt |
| 781 | aaatcagttg tgttcatgag gtgattgtga tttaatttgt tgactagggc gattccttca |
| 841 | catcacaata acaaagtttt atagattttt tttttataac atttttgcca cgcttcgtaa |
| 901 | agtttggtat ttacaccgca tttttccctg tacaagaatt catatattat ttatttatat |
| 961 | actccagttg acaattataa gtttataacg tttttacaat tatttaaata ccatgtgaag |
| 1021 | atccaagaat atgtcttact tcttctttgt gtaagaaaac taactatatc actataataa |
| 1081 | aataattcta atcattatat ttgtaaatat gcagttattt gtcaattttg aatttagtat |
| 1141 | tttagacgtt atcacttcag ccaaatatga tttggattta agtccaaaat gcaatttcgt |
| 1201 | acgtatccct cttgtcgtct aatgattatt tcaatatttc ttatattatc cctaactaca |
| 1261 | gagctacatt tatattgtat tctaatgaca gggaaacttt catagagatt cagatagatg |
| 1321 | aaattggtgg gaaacatcat tgaacaggaa acttttagca aatcatatcg atttatctac |
| 1381 | aaaagaatac ttagcgtaat gaagttcact tgttgtgaat gactatgatt tgatcaaatt |
| 1441 | agttaatttt gtcgaatcat ttttcttttt gatttgatta agcttttaac ttgcacgaat |
| 1501 | ggttctcttg tgaataaaca gaatctttga attcaaacta tttgattagt gaaaagacaa |
| 1561 | aagaagattc cttgttttta tttgattagt gattttgatg catgaaaggt acctacgtac |
| 1621 | tacaagaaaa ataaacatgt acgtaactac gtatcagcat gtaaaagtat ttttttccaa |
| 1681 | ataatttata ctcatgatag attttttttt tttgaaatgt caattaaaaa tgctttctta |
| 1741 | aatattaatt ttaattaatt aaataaggaa atatatttat gcaaaacatc atcaacacat |
| 1801 | atccaacttc gaaaatctct atagtacaca agtagagaaa ttaaatttta ctagatacaa |
| 1861 | acttcctaat catcaaatat aaatgtttac aaaactaatt aaacccacca ctaaaattaa |
| 1921 | ctaaaaatcc gagcaaagtg agtgaacaag acttgatttc aggttgatgt aggactaaaa |
| 1981 | tggctacgta tcaaacatca acgatcattt agttatgtat gaatgaatgt agtcattact |
| 2041 | tgtaaaacaa aaatgctttg atttggatca atcacttcat gtgaacatta gcaattacat |
| 2101 | caaccttatt ttcactataa aaccccatct cagtaccctt ctgaagtaat caaattaaga |
| 2161 | gcaaaagtca tttaactttc ctaaaaca |
| SPG31 promoter, 975 bases | |
| SEQ ID No. 12 | |
| 1 | ATCACTTTAC AAACCAACAA ATGGTGCATC ATCTCAAATG TCATTGTCAA ACCTGTCAGG |
| 61 | ACAAATTATA AAGTACTTGA TCACAAATAC AATTACACAT GGATTTTAAA TAGTCGGACC |
| 121 | GCTGTCCAAA CAAATGACAA TGATGACACG CCATTTGTTC CATGTAAGTG GCTTATTTGT |
| 181 | TTCTACATAC TTTAATTGTA GTTTAGTTAT TTAGAATTTC GTTATGTTTT ATTAAAATAT |
| 241 | ATAAGTATCG AGATTATTTC TTTGATTTTT ATTAATTTAA TATTTTTTCC CTCTCATTAT |
| 301 | TATATGGTTA TTTTAATCAA TTAAGGAACA CTAATTTAAA AATACGCATT AGACATTGAT |
| 361 | TTAATCTCGC AACGCGCGTG TGATAATTAG TAGTTACTAC AAATGCAATA GCATTAGCAG |
| 421 | TTTGGCACCA TTAATTAACG GCTAATGTAC AAAATTAGAG TCCTCCCCTA CCAATTCAAA |
| 481 | GCAACGTCTA CGGAATTCGA ACTTCATTGT CGCTATCATT TGTAAGATAA ATTAGGAGAA |
| 541 | ACAATATTTT GTCGACATAG AGAATAACTG AATATGATTC ATTTATTTAA TTCAATTAAT |
| 601 | ATAACTATTT AAATAAAACT ATTTTTTTTT CAAATTTATC TCACTGTTTA TTAATATAAA |
| 661 | ATAAGTTTAG GAGGAAAATT TAGGATTACT CTAATACGGA GTATTAATTA TTGATTGGCC |
| 721 | GTTAATTAAC GGATAACGCA CAACATTAGA GCCTAGCTTT ATCAAAGCAG CGTCTACGGA |
| 781 | ATTCAAAACT TCATCGTCGC TATCATTTGT AACTTGCCGG CTTTTCCATT GAATCTACCT |
| 841 | CCGTGATAAG ATTCTGTGAC AATCCAGCCC TATAAATACC GTTGCATCTT TAATCTAATG |
| 901 | CATTCACTCA CAACACTTTA CAGTTGTAAA CATTTTACAA CCATTTTTAA TTAAGGTTTC |
| 961 | AGGTTGTCAA TTACT |
| pGC1 promoter, At1g22690, 1163 bases | |
| SEQ ID No. 13 | |
| 1 | atggttgcaa cagagaggat gaatttataa gttttcaaca ccgcttttct tattagacgg |
| 61 | acaacaatct atagtggagt aaatttttat ttttggtaaa atggttagtg aattcaaata |
| 121 | tctaaatttt gtgactcact aacattaaca aatatgcata agacataaaa aaaagaaaga |
| 181 | ataattctta tgaaacaaga aaaaaaacct atacaatcaa tctttaggaa ttgacgatgt |
| 241 | agaattgtag atgataaatt ttctcaaata tagatgggcc taatgaaggg tgccgcttat |
| 301 | tggatctgac ccattttgag gacattaata ttttcattgg ttataagcct tttaatcaaa |
| 361 | attgtcatta aattgatgtc tccctctcgg gtcattttcc tttctccctc acaattaatg |
| 421 | tagactttag caatttgcac gctgtgcttt gtctttatat ttagtaacac aaacattttg |
| 481 | acttgtcttg tagagttttt ctcttttatt tttctatcca atatgaaaac taaaagtgtt |
| 541 | ctcgtataca tatattaaaa ttaaagaaac ctatgaaaac accaatacaa atgcgatatt |
| 601 | gttttcagtt cgacgtttca tgtttgttag aaaatttcta atgacgtttg tataaaatag |
| 661 | acaattaaac gccaaacact acatctgtgt tttcgaacaa tattgcgtct gcgtttcctt |
| 721 | catctatctc tctcagtgtc acaatgtctg aactaagaga cagctgtaaa ctatcattaa |
| 781 | gacataaact accaaagtat caagctaatg taaaaattac tctcatttcc acgtaacaaa |
| 841 | ttgagttagc ttaagatatt agtgaaacta ggtttgaatt ttcttcttct tcttccatgc |
| 901 | atcctccgaa aaaagggaac caataaaaac tgtttgcata tcaaactcca acactttaca |
| 961 | gcaaatgcaa tctataatct gtgatttatc caataaaaac ctgtgattta tgtttggctc |
| 1021 | cagcgatgaa agtctatgca tgtgatctct atccaacatg agtaattgtt cagaaaataa |
| 1081 | aaagtagctg aaatgtatct atataaagaa tcatccacaa gtactatttt cacacactac |
| 1141 | ttcaaaatca ctactcaaaa aat |
| pORER2 35S: GUS, 9192 bases | |
| SEQ ID No. 14 | |
| 1 | GATCGTTCAA ACATTTGGCA ATAAAGTTTC TTAAGATTGA ATCCTGTTGC CGGTCTTGCG |
| 61 | ATGATTATCA TATAATTTCT GTTAATTAAC GTTAAGCATG TAATAATTAA CATGTAATGC |
| 121 | ATGACGTTAT TTATGAGATG GGTTTTTATG ATTAGAGTCC CGCAATTATA CATTTAATAC |
| 181 | GCGATAGAAA ACAAAATATA GCGCGCAAAC TAGGATAAAT TATCGCGCGC GGTGTCATCT |
| 241 | ATGTTACTAG ATCCCTAGGG AAGTTCCTAT TCCGAAGTTC CTATTCTCTG AAAAGTATAG |
| 301 | GAACTTCTTT GCGTATTGGG CGCTCTTGGC CTTTTTGGCC ACCGGTCGTA CGGTTAAAAC |
| 361 | CACCCCAGTA CATTAAAAAC GTCCGCAATG TGTTATTAAG TTGTCTAAGC GTCAATTTGT |
| 421 | TTACACCACA ATATATCCTG CCACCAGCCA GCCAACAGCT CCCCGACCGG CAGCTCGGCA |
| 481 | CAAAATCACC ACTCGATACA GGCAGCCCAT CAGTCCACTA GACGCTCACC GGGCTGGTTG |
| 541 | CCCTCGCCGC TGGGCTGGCG GCCGTCTATG GCCCTGCAAA CGCGCCAGAA ACGCCGTCGA |
| 601 | AGCCGTGTGC GAGACACCGC AGCCGCCGGC GTTGTGGATA CCTCGCGGAA AACTTGGCCC |
| 661 | TCACTGACAG ATGAGGGGCG GACGTTGACA CTTGAGGGGC CGACTCACCC GGCGCGGCGT |
| 721 | TGACAGATGA GGGGCAGGCT CGATTTCGGC CGGCGACGTG GAGCTGGCCA GCCTCGCAAA |
| 781 | TCGGCGAAAA CGCCTGATTT TACGCGAGTT TCCCACAGAT GATGTGGACA AGCCTGGGGA |
| 841 | TAAGTGCCCT GCGGTATTGA CACTTGAGGG GCGCGACTAC TGACAGATGA GGGGCGCGAT |
| 901 | CCTTGACACT TGAGGGGCAG AGTGCTGACA GATGAGGGGC GCACCTATTG ACATTTGAGG |
| 961 | GGCTGTCCAC AGGCAGAAAA TCCAGCATTT GCAAGGGTTT CCGCCCGTTT TTCGGCCACC |
| 1021 | GCTAACCTGT CTTTTAACCT GCTTTTAAAC CAATATTTAT AAACCTTGTT TTTAACCAGG |
| 1081 | GCTGCGCCCT GTGCGCGTGA CCGCGCACGC CGAAGGGGGG TGCCCCCCCT TCTCGAACCC |
| 1141 | TCCCGGCCCG CTCTCGCGTT GGCAGCATCA CCCATAATTG TGGTTTCAAA ATCGGCTCCG |
| 1201 | TCGATACTAT GTTATACGCC AACTTTGAAA ACAACTTTGA AAAAGCTGTT TTCTGGTATT |
| 1261 | TAAGGTTTTA GAATGCAAGG AACAGTGAAT TGGAGTTCGT CTTGTTATAA TTAGCTTCTT |
| 1321 | GGGGTATCTT TAAATACTGT AGAAAAGAGG AAGGAAATAA TAAATGGCTA AAATGAGAAT |
| 1381 | ATCACCGGAA TTGAAAAAAC TGATCGAAAA ATACCGCTGC GTAAAAGATA CGGAAGGAAT |
| 1441 | GTCTCCTGCT AAGGTATATA AGCTGGTGGG AGAAAATGAA AACCTATATT TAAAAATGAC |
| 1501 | GGACAGCCGG TATAAAGGGA CCACCTATGA TGTGGAACGG GAAAAGGACA TGATGCTATG |
| 1561 | GCTGGAAGGA AAGCTGCCTG TTCCAAAGGT CCTGCACTTT GAACGGCATG ATGGCTGGAG |
| 1621 | CAATCTGCTC ATGAGTGAGG CCGATGGCGT CCTTTGCTCG GAAGAGTATG AAGATGAACA |
| 1681 | AAGCCCTGAA AAGATTATCG AGCTGTATGC GGAGTGCATC AGGCTCTTTC ACTCCATCGA |
| 1741 | CATATCGGAT TGTCCCTATA CGAATAGCTT AGACAGCCGC TTAGCCGAAT TGGATTACTT |
| 1801 | ACTGAATAAC GATCTGGCCG ATGTGGATTG CGAAAACTGG GAAGAAGACA CTCCATTTAA |
| 1861 | AGATCCGCGC GAGCTGTATG ATTTTTTAAA GACGGAAAAG CCCGAAGAGG AACTTGTCTT |
| 1921 | TTCCCACGGC GACCTGGGAG ACAGCAACAT CTTTGTGAAA GATGGCAAAG TAAGTGGCTT |
| 1981 | TATTGATCTT GGGAGAAGCG GCAGGGCGGA CAAGTGGTAT GACATTGCCT TCTGCGTCCG |
| 2041 | GTCGATCAGG GAGGATATTG GGGAAGAACA GTATGTCGAG CTATTTTTTG ACTTACTGGG |
| 2101 | GATCAAGCCT GATTGGGAGA AAATAAAATA TTATATTTTA CTGGATGAAT TGTTTTAGTA |
| 2161 | CCTAGATGTG GCGCAACGAT GCCGGCGACA AGCAGGAGCG CACCGACTTC TTCCGCATCA |
| 2221 | AGTGTTTTGG CTCTCAGGCC GAGGCCCACG GCAAGTATTT GGGCAAGGGG TCGCTGGTAT |
| 2281 | TCGTGCAGGG CAAGATTCGG AATACCAAGT ACGAGAAGGA CGGCCAGACG GTCTACGGGA |
| 2341 | CCGACTTCAT TGCCGATAAG GTGGATTATC TGGACACCAA GGCACCAGGC GGGTCAAATC |
| 2401 | AGGAATAAGG GCACATTGCC CCGGCGTGAG TCGGGGCAAT CCCGCAAGGA GGGTGAATGA |
| 2461 | ATCGGACGTT TGACCGGAAG GCATACAGGC AAGAACTGAT CGACGCGGGG TTTTCCGCCG |
| 2521 | AGGATGCCGA AACCATCGCA AGCCGCACCG TCATGCGTGC GCCCCGCGAA ACCTTCCAGT |
| 2581 | CCGTCGGCTC GATGGTCCAG CAAGCTACGG CCAAGATCGA GCGCGACAGC GTGCAACTGG |
| 2641 | CTCCCCCTGC CCTGCCCGCG CCATCGGCCG CCGTGGAGCG TTCGCGTCGT CTCGAACAGG |
| 2701 | AGGCGGCAGG TTTGGCGAAG TCGATGACCA TCGACACGCG AGGAACTATG ACGACCAAGA |
| 2761 | AGCGAAAAAC CGCCGGCGAG GACCTGGCAA AACAGGTCAG CGAGGCCAAG CAAGCCGCGT |
| 2821 | TGCTGAAACA CACGAAGCAG CAGATCAAGG AAATGCAGCT TTCCTTGTTC GATATTGCGC |
| 2881 | CGTGGCCGGA CACGATGCGA GCGATGCCAA ACGACACGGC CCGCTCTGCC CTGTTCACCA |
| 2941 | CGCGCAACAA GAAAATCCCG CGCGAGGCGC TGCAAAACAA GGTCATTTTC CACGTCAACA |
| 3001 | AGGACGTGAA GATCACCTAC ACCCGCGTCG AGCTGCGGGC CGACGATGAC GAACTGGTGT |
| 3061 | GGCAGCAGGT GTTGGAGTAC GCGAAGCGCA CCCCTATCGG CGAGCCGATC ACCTTCACGT |
| 3121 | TCTACGAGCT TTGCCAGGAC CTGGGCTGGT CGATCAATGG CCGGTATTAC ACGAAGGCCG |
| 3181 | AGGAATGCCT GTCGCGCCTA CAGGCGACGG CGATGGGCTT CACGTCCGAC CGCGTTGGGC |
| 3241 | ACCTGGAATC GGTGTCGCTG CTGCACCGCT TCCGCGTCCT GGACCGTGGC AAGAAAACGT |
| 3301 | CCCGTTGCCA GGTCCTGATC GACGAGGAAA TCGTCGTGCT GTTTGCTGGC GACCACTACA |
| 3361 | CGAAATTCAT ATGGGAGAAG TACCGCAAGC TGTCGCCGAC GGCCCGACGG ATGTTCGACT |
| 3421 | ATTTCAGCTC GCACCGGGAG CCGTACCCGC TCAAGCTGGA AACCTTCCGC CTCATGTGCG |
| 3481 | GATCGGATTC CACCCGCGTG AAGAAGTGGC GCGAGCAGGT CGGCGAAGCC TGCGAAGAGT |
| 3541 | TGCGAGGCAG CGGCCTGGTG GAACACGCCT GGGTCAATGA TGACCTGGTG CATTGCAAAC |
| 3601 | GCTAGGGCCT TGTGGGGTCA GTTCCGGCTG GGGGTTCAGC AGCCAGCGCT TTACTGAGAT |
| 3661 | CCTCTTCCGC TTCCTCGCTC ACTGACTCGC TGCGCTCGGT CGTTCGGCTG CGGCGAGCGG |
| 3721 | TATCAGCTCA CTCAAAGGCG GTAATACGGT TATCCACAGA ATCAGGGGAT AACGCAGGAA |
| 3781 | AGAACATGTG AGCAAAAGGC CAGCAAAAGG CCAGGAACCG TAAAAAGGCC GCGTTGCTGG |
| 3841 | CGTTTTTCCA TAGGCTCCGC CCCCCTGACG AGCATCACAA AAATCGACGC TCAAGTCAGA |
| 3901 | GGTGGCGAAA CCCGACAGGA CTATAAAGAT ACCAGGCGTT TCCCCCTGGA AGCTCCCTCG |
| 3961 | TGCGCTCTCC TGTTCCGACC CTGCCGCTTA CCGGATACCT GTCCGCCTTT CTCCCTTCGG |
| 4021 | GAAGCGTGGC GCTTTCTCAT AGCTCACGCT GTAGGTATCT CAGTTCGGTG TAGGTCGTTC |
| 4081 | GCTCCAAGCT GGGCTGTGTG CACGAACCCC CCGTTCAGCC CGACCGCTGC GCCTTATCCG |
| 4141 | GTAACTATCG TCTTGAGTCC AACCCGGTAA GACACGACTT ATCGCCACTG GCAGCAGCCA |
| 4201 | CTGGTAACAG GATTAGCAGA GCGAGGTATG TAGGCGGTGC TACAGAGTTC TTGAAGTGGT |
| 4261 | GGCCTAACTA CGGCTACACT AGAAGAACAG TATTTGGTAT CTGCGCTCTG CTGAAGCCAG |
| 4321 | TTACCTTCGG AAAAAGAGTT GGTAGCTCTT GATCCGGCAA ACAAACCACC GCTGGTAGCG |
| 4381 | GTGGTTTTTT TGTTTGCAAG CAGCAGATTA CGCGCAGAAA AAAAGGATCT CAAGAAGATC |
| 4441 | CTTTGATCTT TTCTACGGGG TCTGACGCTC AGTGGAACGA AAACTCACGT TAAGGGATTT |
| 4501 | TGGTCATGAG ATTATCAAAA AGGATCTTCA CCTAGATCCT TTTGGATCTC CTGTGGTTGG |
| 4561 | CATGCACATA CAAATGGACG AACGGATAAA CCTTTTCACG CCCTTTTAAA TATCCGATTA |
| 4621 | TTCTAATAAA CGCTCTTTTC TCTTAGGTTT ACCCGCCAAT ATATCCTGTC AAACACTGAT |
| 4681 | AGTTTAAACT GAAGGCGGGA AACGACAATC TGCTAGTGGA TCTCCCAGTC ACGACGTTGT |
| 4741 | AAAACGGGCG CCCCGCGGAA AGCTTGCATG CCTGCAGGTC CCCAGATTAG CCTTTTCAAT |
| 4801 | TTCAGAAAGA ATGCTAACCC ACAGATGGTT AGAGAGGCTT ACGCAGCAGG TCTCATCAAG |
| 4861 | ACGATCTACC CGAGCAATAA TCTCCAGGAA ATCAAATACC TTCCCAAGAA GGTTAAAGAT |
| 4921 | GCAGTCAAAA GATTCAGGAC TAACTGCATC AAGAACACAG AGAAAGATAT ATTTCTCAAG |
| 4981 | ATCAGAAGTA CTATTCCAGT ATGGACGATT CAAGGCTTGC TTCACAAACC AAGGCAAGTA |
| 5041 | ATAGAGATTG GAGTCTCTAA AAAGGTAGTT CCCACTGAAT CAAAGGCCAT GGAGTCAAAG |
| 5101 | ATTCAAATAG AGGACCTAAC AGAACTCGCC GTAAAGACTG GCGAACAGTT CATACAGAGT |
| 5161 | CTCTTACGAC TCAATGACAA GAAGAAAATC TTCGTCAACA TGGTGGAGCA CGACACACTT |
| 5221 | GTCTACTCCA AAAATATCAA AGATACAGTC TCAGAAGACC AAAGGGCAAT TGAGACTTTT |
| 5281 | CAACAAAGGG TAATATCCGG AAACCTCCTC GGATTCCATT GCCCAGCTAT CTGTCACTTT |
| 5341 | ATTGTGAAGA TAGTGGAAAA GGAAGGTGGC TCCTACAAAT GCCATCATTG CGATAAAGGA |
| 5401 | AAGGCCATCG TTGAAGATGC CTCTGCCGAC AGTGGTCCCA AAGATGGACC CCCACCCACG |
| 5461 | AGGAGCATCG TGGAAAAAGA AGACGTTCCA ACCAGGCGTT CAAAGCAAGT GGATTGATGT |
| 5521 | GATATCTCCA CTGACGTAAG GGATGACGCA CAATCCCACT ATCCTTCGCA AGACCCTTCC |
| 5581 | TCTATATAAG GAAGTTCATT TCATTTGGAG AGAACACGGG GGACTCTAGA AGGCCTTGGA |
| 5641 | TCCACCCGGG AGAATTCGTC GACTTTGCGG CCGCATCGAT ACTGCAGGAG CTCATGTTAC |
| 5701 | GTCCTGTAGA AACCCCAACC CGTGAAATCA AAAAACTCGA CGGCCTGTGG GCATTCAGTC |
| 5761 | TGGATCGCGA AAACTGTGGA ATTGATCAGC GTTGGTGGGA AAGCGCGTTA CAAGAAAGCC |
| 5821 | GGGCTATTGC TGTGCCAGGC AGTTTTAACG ATCAGTTCGC CGATGCAGAT ATTCGTAATT |
| 5881 | ATGCGGGCAA CGTCTGGTAT CAGCGCGAAG TCTTTATACC GAAAGGTTGG GCAGGCCAGC |
| 5941 | GTATCGTGCT GCGTTTCGAT GCGGTCACTC ATTACGGCAA AGTGTGGGTC AATAATCAGG |
| 6001 | AAGTGATGGA GCATCAGGGC GGCTATACGC CATTTGAAGC CGATGTCACG CCGTATGTTA |
| 6061 | TTGCCAGGAA AAGTGTACGT ATCACCGTTT GTGTGAACAA CGAACTGAAC TGGCAGACTA |
| 6121 | TCCCGCCGGG AATGGTGATT ACCGACGAAA ACGGCAAGAA AAAGCAGTCT TACTTCCATG |
| 6181 | ATTTCTTTAA CTATGCCGGA ATCCATCGCA GCGTAATGCT CTACACCACG CCGAACACCT |
| 6241 | GGGTGGACGA TATTACCGTG GTGACGCATG TCGCGCAAGA CTGTAACCAC GCTTCTGTTG |
| 6301 | ACTGGCAGGT GGTGGCCAAT GGTGATGTCA GCGTTGAACT GCGTGATGCG GATCAACAGG |
| 6361 | TGGTTGCAAC TGGACAAGGC ACTAGCGGGA CTTTGCAAGT GGTGAATCCG CACCTCTGGC |
| 6421 | AACCGGGTGA AGGTTATCTC TATGAACTGT GCGTCACAGC CAAAAGCCAG ACAGAGTGTG |
| 6481 | ATATTTACCC GCTTCGCGTC GGCATCCGGT CAGTGGCAGT GAAGGGCGAA CAGTTCCTGA |
| 6541 | TTAACCACAA ACCGTTCTAC TTTACTGGCT TTGGTCGTCA TGAAGATGCG GACTTGCGTG |
| 6601 | GCAAAGGATT CGATAACGTG CTGATGGTGC ACGACCACGC ATTAATGGAC TGGATTGGGG |
| 6661 | CCAACTCCTA CCGTACCTCG CATTACCCTT ACGCTGAAGA GATGCTCGAC TGGGCAGATG |
| 6721 | AACATGGCAT CGTGGTGATT GATGAAACTG CTGCTGTCGG CTTTAACCTC TCTTTAGGCA |
| 6781 | TTGGTTTCGA AGCGGGCAAC AAGCCGAAAG AACTGTACAG CGAAGAGGCA GTCAACGGGG |
| 6841 | AAACTCAGCA AGCGCACTTA CAGGCGATTA AAGAGCTGAT AGCGCGTGAC AAAAACCACC |
| 6901 | CAAGCGTGGT GATGTGGAGT ATTGCCAACG AACCGGATAC CCGTCCGCAA GGTGCACGGG |
| 6961 | AATATTTCGC GCCACTGGCG GAAGCAACCC GTAAACTCGA CCCGACCCGT CCGATCACCT |
| 7021 | GCGTCAATGT AATGTTCTGC GACGCTCACA CCGATACCAT CAGCGATCTC TTTGATGTGC |
| 7081 | TGTGCCTGAA CCGTTATTAC GGATGGTATG TCCAAAGCGG CGATTTGGAA ACGGCAGAGA |
| 7141 | AGGTACTGGA AAAAGAACTT CTGGCCTGGC AGGAGAAACT GCATCAGCCG ATTATCATCA |
| 7201 | CCGAATACGG CGTGGATACG TTAGCCGGGC TGCACTCAAT GTACACCGAC ATGTGGAGTG |
| 7261 | AAGAGTATCA GTGTGCATGG CTGGATATGT ATCACCGCGT CTTTGATCGC GTCAGCGCCG |
| 7321 | TCGTCGGTGA ACAGGTATGG AATTTCGCCG ATTTTGCGAC CTCGCAAGGC ATATTGCGCG |
| 7381 | TTGGCGGTAA CAAGAAAGGG ATCTTCACTC GCGACCGCAA ACCGAAGTCG GCGGCTTTTC |
| 7441 | TGCTGCAAAA ACGCTGGACT GGCATGAACT TCGGTGAAAA ACCGCAGCAG GGAGGCAAAC |
| 7501 | AATGAGGTAC CTTTTACTAG TGATATCCCT GTGTGAAATT GTTATCCGCT ACGCGTGATC |
| 7561 | GTTCAAACAT TTGGCAATAA AGTTTCTTAA GATTGAATCC TGTTGCCGGT CTTGCGATGA |
| 7621 | TTATCATATA ATTTCTGTTG AATTACGTTA AGCATGTAAT AATTAACATG TAATGCATGA |
| 7681 | CGTTATTTAT GAGATGGGTT TTTATGATTA GAGTCCCGCA ATTATACATT TAATACGCGA |
| 7741 | TAGAAAACAA AATATAGCGC GCAAACTAGG ATAAATTATC GCGCGCGGTG TCATCTATGT |
| 7801 | TACTAGATCC CATGGGAAGT TCCTATTCCG AAGTTCCTAT TCTCTGAAAA GTATAGGAAC |
| 7861 | TTCAGCGATC GCAGACGTCG GGATCTTCTG CAAGCATCTC TATTTCCTGA AGGTCTAACC |
| 7921 | TCGAAGATTT AAGATTTAAT TACGTTTATA ATTACAAAAT TGATTCTAGT ATCTTTAATT |
| 7981 | TAATGCTTAT ACATTATTAA TTAATTTAGT ACTTTCAATT TGTTTTCAGA AATTATTTTA |
| 8041 | CTATTTTTTA TAAAATAAAA GGGAGAAAAT GGCTATTTAA ATACTAGCCT ATTTTATTTC |
| 8101 | AATTTTAGCT TAAAATCAGC CCCAATTAGC CCCAATTTCA AATTCAAATG GTCCAGCCCA |
| 8161 | ATTCCTAAAT AACCCACCCC TAACCCGCCC GGTTTCCCCT TTTGATCCAT GCAGTCAACG |
| 8221 | CCCAGAATTT CCCTATATAA TTTTTTAATT CCCAAACACC CCTAACTCTA TCCCATTTCT |
| 8281 | CACCAACCGC CACATAGATC TATCCTCTTA TCTCTCAAAC TCTCTCGAAC CTTCCCCTAA |
| 8341 | CCCTAGCAGC CTCTCATCAT CCTCACCTCA AAACCCACCG GGGCCGGCCA TCATTGAACA |
| 8401 | AGATGGATTG CACGCAGGTT CTCCGGCCGC TTGGGTGGAG AGGCTATTCG GCTATGACTG |
| 8461 | GGCACAACAG ACAATCGGCT GCTCTGATGC CGCCGTGTTC CGGCTGTCAG CGCAGGGGAG |
| 8521 | GCCGGTTCTT TTTGTCAAGA CCGACCTGTC CGGTGCCCTG AATGAACTTC AAGACGAGGC |
| 8581 | AGCGCGGCTA TCGTGGCTGG CCACGACGGG CGTTCCTTGC GCAGCTGTGC TCGACGTTGT |
| 8641 | CACTGAAGCG GGAAGGGACT GGCTGCTATT GGGCGAAGTG CCGGGGCAGG ATCTCCTGTC |
| 8701 | ATCTCACCTT GCTCCTGCCG AGAAAGTATC CATCATGGCT GATGCAATGC GGCGGCTGCA |
| 8761 | TACGCTTGAT CCGGCTACCT GCCCATTCGA CCACCAAGCG AAACATCGCA TCGAGCGAGC |
| 8821 | ACGTACTCGG ATGGAAGCCG GTCTTGTCGA TCAGGATGAT CTGGACGAAG AGCATCAGGG |
| 8881 | GCTCGCGCCA GCCGAACTGT TCGCCAGGCT CAAGGCGCGC ATGCCCGACG GCGAGGATCT |
| 8941 | CGTCGTGACT CATGGCGATG CCTGCTTGCC GAATATCATG GTGGAAAATG GCCGCTTTTC |
| 9001 | TGGATTCATC GACTGTGGCC GGCTGGGTGT GGCGGACCGC TATCAGGACA TAGCGTTGGC |
| 9061 | TACCCGTGAT ATTGCTGAAG AGCTTGGCGG CGAATGGGCT GACCGCTTCC TCGTGCTTTA |
| 9121 | CGGTATCGCC GCTCCCGATT CGCAGCGCAT CGCCTTCTAT CGCCTTCTTG ACGAGTTCTT |
| 9181 | CTGAGGCGCG CC |
| pORE_R2 35S: StAKIP1/2, 9041 bases | |
| SEQ ID No. 15 | |
| 1 | GATCGTTCAA ACATTTGGCA ATAAAGTTTC TTAAGATTGA ATCCTGTTGC CGGTCTTGCG |
| 61 | ATGATTATCA TATAATTTCT GTTGAATTAC GTTAAGCATG TAATAATTAA CATGTAATGC |
| 121 | ATGACGTTAT TTATGAGATG GGTTTTTATG ATTAGAGTCC CGCAATTATA CATTTAATAC |
| 181 | GCGATAGAAA ACAAAATATA GCGCGCAAAC TAGGATAAAT TATCGCGCGC GGTGTCATCT |
| 241 | ATGTTACTAG ATCCCTAGGG AAGTTCCTAT TCCGAAGTTC CTATTCTCTG AAAAGTATAG |
| 301 | GAACTTCTTT GCGTATTGGG CGCTCTTGGC CTTTTTGGCC ACCGGTCGTA CGGTTAAAAC |
| 361 | CACCCCAGTA CATTAAAAAC GTCCGCAATG TGTTATTAAG TTGTCTAAGC GTCAATTTGT |
| 421 | TTACACCACA ATATATCCTG CCACCAGCCA GCCAACAGCT CCCCGACCGG CAGCTCGGCA |
| 481 | CAAAATCACC ACTCGATACA GGCAGCCCAT CAGTCCACTA GACGCTCACC GGGCTGGTTG |
| 541 | CCCTCGCCGC TGGGCTGGCG GCCGTCTATG GCCCTGCAAA CGCGCCAGAA ACGCCGTCGA |
| 601 | AGCCGTGTGC GAGACACCGC AGCCGCCGGC GTTGTGGATA CCTCGCGGAA AACTTGGCCC |
| 661 | TCACTGACAG ATGAGGGGCG GACGTTGACA CTTGAGGGGC CGACTCACCC GGCGCGGCGT |
| 721 | TGACAGATGA GGGGCAGGCT CGATTTCGGC CGGCGACGTG GAGCTGGCCA GCCTCGCAAA |
| 781 | TCGGCGAAAA CGCCTGATTT TACGCGAGTT TCCCACAGAT GATGTGGACA AGCCTGGGGA |
| 841 | TAAGTGCCCT GCGGTATTGA CACTTGAGGG GCGCGACTAC TGACAGATGA GGGGCGCGAT |
| 901 | CCTTGACACT TGAGGGGCAG AGTGCTGACA GATGAGGGGC GCACCTATTG ACATTTGAGG |
| 961 | GGCTGTCCAC AGGCAGAAAA TCCAGCATTT GCAAGGGTTT CCGCCCGTTT TTCGGCCACC |
| 1021 | GCTAACCTGT CTTTTAACCT GCTTTTAAAC CAATATTTAT AAACCTTGTT TTTAACCAGG |
| 1081 | GCTGCGCCCT GTGCGCGTGA CCGCGCACGC CGAAGGGGGG TGCCCCCCCT TCTCGAACCC |
| 1141 | TCCCGGCCCG CTCTCGCGTT GGCAGCATCA CCCATAATTG TGGTTTCAAA ATCGGCTCCG |
| 1201 | TCGATACTAT GTTATACGCC AACTTTGAAA ACAACTTTGA AAAAGCTGTT TTCTGGTATT |
| 1261 | TAAGGTTTTA GAATGCAAGG AACAGTGAAT TGGAGTTCGT CTTGTTATAA TTAGCTTCTT |
| 1321 | GGGGTATCTT TAAATACTGT AGAAAAGAGG AAGGAAATAA TAAATGGCTA AAATGAGAAT |
| 1381 | ATCACCGGAA TTGAAAAAAC TGATCGAAAA ATACCGCTGC GTAAAAGATA CGGAAGGAAT |
| 1441 | GTCTCCTGCT AAGGTATATA AGCTGGTGGG AGAAAATGAA AACCTATATT TAAAAATGAC |
| 1501 | GGACAGCCGG TATAAAGGGA CCACCTATGA TGTGGAACGG GAAAAGGACA TGATGCTATG |
| 1561 | GCTGGAAGGA AAGCTGCCTG TTCCAAAGGT CCTGCACTTT GAACGGCATG ATGGCTGGAG |
| 1621 | CAATCTGCTC ATGAGTGAGG CCGATGGCGT CCTTTGCTCG GAAGAGTATG AAGATGAACA |
| 1681 | AAGCCCTGAA AAGATTATCG AGCTGTATGC GGAGTGCATC AGGCTCTTTC ACTCCATCGA |
| 1741 | CATATCGGAT TGTCCCTATA CGAATAGCTT AGACAGCCGC TTAGCCGAAT TGGATTACTT |
| 1801 | ACTGAATAAC GATCTGGCCG ATGTGGATTG CGAAAACTGG GAAGAAGACA CTCCATTTAA |
| 1861 | AGATCCGCGC GAGCTGTATG ATTTTTTAAA GACGGAAAAG CCCGAAGAGG AACTTGTCTT |
| 1921 | TTCCCACGGC GACCTGGGAG ACAGCAACAT CTTTGTGAAA GATGGCAAAG TAAGTGGCTT |
| 1981 | TATTGATCTT GGGAGAAGCG GCAGGGCGGA CAAGTGGTAT GACATTGCCT TCTGCGTCCG |
| 2041 | GTCGATCAGG GAGGATATTG GGGAAGAACA GTATGTCGAG CTATTTTTTG ACTTACTGGG |
| 2101 | GATCAAGCCT GATTGGGAGA AAATAAAATA TTATATTTTA CTGGATGAAT TGTTTTAGTA |
| 2161 | CCTAGATGTG GCGCAACGAT GCCGGCGACA AGCAGGAGCG CACCGACTTC TTCCGCATCA |
| 2221 | AGTGTTTTGG CTCTCAGGCC GAGGCCCACG GCAAGTATTT GGGCAAGGGG TCGCTGGTAT |
| 2281 | TCGTGCAGGG CAAGATTCGG AATACCAAGT ACGAGAAGGA CGGCCAGACG GTCTACGGGA |
| 2341 | CCGACTTCAT TGCCGATAAG GTGGATTATC TGGACACCAA GGCACCAGGC GGGTCAAATC |
| 2401 | AGGAATAAGG GCACATTGCC CCGGCGTGAG TCGGGGCAAT CCCGCAAGGA GGGTGAATGA |
| 2461 | ATCGGACGTT TGACCGGAAG GCATACAGGC AAGAACTGAT CGACGCGGGG TTTTCCGCCG |
| 2521 | AGGATGCCGA AACCATCGCA AGCCGCACCG TCATGCGTGC GCCCCGCGAA ACCTTCCAGT |
| 2581 | CCGTCGGCTC GATGGTCCAG CAAGCTACGG CCAAGATCGA GCGCGACAGC GTGCAACTGG |
| 2641 | CTCCCCCTGC CCTGCCCGCG CCATCGGCCG CCGTGGAGCG TTCGCGTCGT CTCGAACAGG |
| 2701 | AGGCGGCAGG TTTGGCGAAG TCGATGACCA TCGACACGCG AGGAACTATG ACGACCAAGA |
| 2761 | AGCGAAAAAC CGCCGGCGAG GACCTGGCAA AACAGGTCAG CGAGGCCAAG CAAGCCGCGT |
| 2821 | TGCTGAAACA CACGAAGCAG CAGATCAAGG AAATGCAGCT TTCCTTGTTC GATATTGCGC |
| 2881 | CGTGGCCGGA CACGATGCGA GCGATGCCAA ACGACACGGC CCGCTCTGCC CTGTTCACCA |
| 2941 | CGCGCAACAA GAAAATCCCG CGCGAGGCGC TGCAAAACAA GGTCATTTTC CACGTCAACA |
| 3001 | AGGACGTGAA GATCACCTAC ACCGGCGTCG AGCTGCGGGC CGACGATGAC GAACTGGTGT |
| 3061 | GGCAGCAGGT GTTGGAGTAC GCGAAGCGCA CCCCTATCGG CGAGCCGATC ACCTTCACGT |
| 3121 | TCTACGAGCT TTGCCAGGAC CTGGGCTGGT CGATCAATGG CCGGTATTAC ACGAAGGCCG |
| 3181 | AGGAATGCCT GTCGCGCCTA CAGGCGACGG CGATGGGCTT CACGTCCGAC CGCGTTGGGC |
| 3241 | ACCTGGAATC GGTGTCGCTG CTGCACCGCT TCCGCGTCCT GGACCGTGGC AAGAAAACGT |
| 3301 | CCCGTTGCCA GGTCCTGATC GACGAGGAAA TCGTCGTGCT GTTTGCTGGC GACCACTACA |
| 3361 | CGAAATTCAT ATGGGAGAAG TACCGCAAGC TGTCGCCGAC GGCCCGACGG ATGTTCGACT |
| 3421 | ATTTCAGCTC GCACCGGGAG CCGTACCCGC TCAAGCTGGA AACCTTCCGC CTCATGTGCG |
| 3481 | GATCGGATTC CACCCGCGTG AAGAAGTGGC GCGAGCAGGT CGGCGAAGCC TGCGAAGAGT |
| 3541 | TGCGAGGCAG CGGCCTGGTG GAACACGCCT GGGTCAATGA TGACCTGGTG CATTGCAAAC |
| 3601 | GCTAGGGCCT TGTGGGGTCA GTTCCGGCTG GGGGTTCAGC AGCCAGCGCT TTACTGAGAT |
| 3661 | CCTCTTCCGC TTCCTCGCTC ACTGACTCGC TGCGCTCGGT CGTTCGGCTG CGGCGAGCGG |
| 3721 | TATCAGCTCA CTCAAAGGCG GTAATACGGT TATCCACAGA ATCAGGGGAT AACGCAGGAA |
| 3781 | AGAACATGTG AGCAAAAGGC CAGCAAAAGG CCAGGAACCG TAAAAAGGCC GCGTTGCTGG |
| 3841 | CGTTTTTCCA TAGGCTCCGC CCCCCTGACG AGCATCACAA AAATCGACGC TCAAGTCAGA |
| 3901 | GGTGGCGAAA CCCGACAGGA CTATAAAGAT ACCAGGCGTT TCCCCCTGGA AGCTCCCTCG |
| 3961 | TGCGCTCTCC TGTTCCGACC CTGCCGCTTA CCGGATACCT GTCCGCCTTT CTCCCTTCGG |
| 4021 | GAAGCGTGGC GCTTTCTCAT AGCTCACGCT GTAGGTATCT CAGTTCGGTG TAGGTCGTTC |
| 4081 | GCTCCAAGCT GGGCTGTGTG CACGAACCCC CCGTTCAGCC CGACCGCTGC GCCTTATCCG |
| 4141 | GTAACTATCG TCTTGAGTCC AACCCGGTAA GACACGACTT ATCGCCACTG GCAGCAGCCA |
| 4201 | CTGGTAACAG GATTAGCAGA GCGAGGTATG TAGGCGGTGC TACAGAGTTC TTGAAGTGGT |
| 4261 | GGCCTAACTA CGGCTACACT AGAAGAACAG TATTTGGTAT CTGCGCTCTG CTGAAGCCAG |
| 4321 | TTACCTTCGG AAAAAGAGTT GGTAGCTCTT GATCCGGCAA ACAAACCACC GCTGGTAGCG |
| 4381 | GTGGTTTTTT TGTTTGCAAG CAGCAGATTA CGCGCAGAAA AAAAGGATCT CAAGAAGATC |
| 4441 | CTTTGATCTT TTCTACGGGG TCTGACGCTC AGTGGAACGA AAACTCACGT TAAGGGATTT |
| 4501 | TGGTCATGAG ATTATCAAAA AGGATCTTCA CCTAGATCCT TTTGGATCTC CTGTGGTTGG |
| 4561 | CATGCACATA CAAATGGACG AACGGATAAA CCTTTTCACG CCCTTTTAAA TATCCGATTA |
| 4621 | TTCTAATAAA CGCTCTTTTC TCTTAGGTTT ACCCGCCAAT ATATCCTGTC AAACACTGAT |
| 4681 | AGTTTAAACT GAAGGCGGGA AACGACAATC TGCTAGTGGA TCTCCCAGTC ACGACGTTGT |
| 4741 | AAAACGGGCG CCCCGCGGAA AGCTTGCATG CCTGCAGGTC CCCAGATTAG CCTTTTCAAT |
| 4801 | TTCAGAAAGA ATGCTAACCC ACAGATGGTT AGAGAGGCTT ACGCAGCAGG TCTCATCAAG |
| 4861 | ACGATCTACC CGAGCAATAA TCTCCAGGAA ATCAAATACC TTCCCAAGAA GGTTAAAGAT |
| 4921 | GCAGTCAAAA GATTCAGGAC TAACTGCATC AAGAACACAG AGAAAGATAT ATTTCTCAAG |
| 4981 | ATCAGAAGTA CTATTCCAGT ATGGACGATT CAAGGCTTGC TTCACAAACC AAGGCAAGTA |
| 5041 | ATAGAGATTG GAGTCTCTAA AAAGGTAGTT CCCACTGAAT CAAAGGCCAT GGAGTCAAAG |
| 5101 | ATTCAAATAG AGGACCTAAC AGAACTCGCC GTAAAGACTG GCGAACAGTT CATACAGAGT |
| 5161 | CTCTTACGAC TCAATGACAA GAAGAAAATC TTCGTCAACA TGGTGGAGCA CGACACACTT |
| 5221 | GTCTACTCCA AAAATATCAA AGATACAGTC TCAGAAGACC AAAGGGCAAT TGAGACTTTT |
| 5281 | CAACAAAGGG TAATATCCGG AAACCTCCTC GGATTCCATT GCCCAGCTAT CTGTCACTTT |
| 5341 | ATTGTGAAGA TAGTGGAAAA GGAAGGTGGC TCCTACAAAT GCCATCATTG CGATAAAGGA |
| 5401 | AAGGCCATCG TTGAAGATGC CTCTGCCGAC AGTGGTCCCA AAGATGGACC CCCACCCACG |
| 5461 | AGGAGCATCG TGGAAAAAGA AGACGTTCCA ACCACGTCTT CAAAGCAAGT GGATTGATGT |
| 5521 | GATATCTCCA CTGACGTAAG GGATGACGCA CAATCCCACT ATCCTTCGCA AGACCCTTCC |
| 5581 | TCTATATAAG GAAGTTCATT TCATTTGGAG AGAACACGGG GGACTCTAGA AGGCCTTGGA |
| 5641 | TCCACCCGGG AGAATTCGTC GACTTTGCGG CCGCATCGAT ACTGCAGGAG CTCGGATCCA |
| 5701 | CTAGTAACGG CCGCCAGTGT GCTGGAATTC GCCCTTCCAA ATCAGAAAAC CCTAATTTCA |
| 5761 | ACTATTTCTC TCTCTATACC TCAATATTCC CATGGCGAAG AAGCGAAAAA CCAGAGCTTC |
| 5821 | CGAACCTTCA CAACCAGTTG AAGAACCAGT AGAGATTAAG CAAGAACCTG AGGTTGAAAC |
| 5881 | CGCACAAGAA GAAGAATACG AGAACGATGC CGTAGAAGTA GAGGAAGAAG AAGCAGTACA |
| 5941 | TGAGGACGAA GAAGAACAAG ATCCCGAAGA GGAAGACGAA GAAGGCGGTG ACGATGAGGA |
| 6001 | GGAAGAAGAA GAAATGCCCG GATCTGGAAA TGACGCTGTA GAGGATGATA ACGAGAAGTC |
| 6061 | GGTTAATGAA CAGGAACAAG TTAATGGAGG CCCTGAGGTG AAGATGGAGG AAGGTAATGA |
| 6121 | AGAGGATTTG GAAGATGAGC CACTTGAGAA TCTGTTAGAG CCATTTACGA AGGATCAACT |
| 6181 | TACAGCGCTT ATTAAAGAAG CTTTAGCTAA ATACCCTGAT TTCAAAGAAA ATATTCAGAA |
| 6241 | ATTGGCTGAT AAAGATCCGG CACACCGGAA AATCTTTGTC CACGGTTTAG GTTGGGATAC |
| 6301 | GACAGCTGAA ACGCTAACTA GTGTTTTTGC AACTTACGGT GAAATTGAGG ATTGTAAAGC |
| 6361 | TGTTACAGAT AAGGTTTCGG GGAAATCGAA AGGTTATGGA TTTATATTGT TTAAGCATAG |
| 6421 | GAGTGGTGCT AGGAAAGCTT TGAAAGAACC ACAGAAGAAG ATTGGTACAA GGATGACTTC |
| 6481 | TTGCCAGTTG GCTTCTGCTG GGCCGGTTCC GGCTCCTCCA CCTACTACAG CAGCCCCACC |
| 6541 | GGTTTCGGAG TACACACAGA GGAAAATTTT TGTTAGCAAT GTAGCTGCTG ATCTTGAACC |
| 6601 | TCAAAAGTTG TTGGAGTACT TCTCAAAGTT TGGTGAAGTT GAGGAGGGTC CACTTGGATT |
| 6661 | GGATAAGCAA AATGGGAAAC CTAAGGGGTT TTGTTTGTTT GTGTATAAGA CTGTTGAGGG |
| 6721 | TGCTAGGAAG GCATTGGAGG AGCCACATAA GACCTTTGAA GGGCATACCC TTCATTGTCA |
| 6781 | GAAGGCGATT GATGGACCGA AGCATAGTAA GAATTTCCAT CAGCAGCAAC AGCCTCAGTC |
| 6841 | ACAACAGCAT TACCCTCAGC ATCATCATCA TAATCAGCAG CAGCATTATT ATCAGCATCC |
| 6901 | TGCAAAGAAG GGGAAATATT CAGGTAGCAG TGCAGGAGCA GGATCAGCTG GGCATTTAAT |
| 6961 | GGCACCATCG GGGCCTGCAC CTGTGGGATA CAATCCTGCT GTGGCAGCTG TGACCCCAGC |
| 7021 | TCTTGGACAG GCACTGACAG CCCTGTTAGC TACACAAGGA GCTGGTTTGG GGATTGGGAA |
| 7081 | TTTGCTTGGA GGTTTTGGTG GGCCAGTGAA TCATCAGGGT GTGCAACCAG TGGTGAACAA |
| 7141 | TGCAACAGGA TATGGTGCCC AGGGTGGATA TGGAGCTCAG CCTCATATGC AATACCAAAA |
| 7201 | CCCTCAGATG CAGGGTGGTG CAAGACCACA AGGTGGTGGT GCCCCATACT CTGGCTATGG |
| 7261 | AGGTCACTAG GAAAACTTAA GGTATCTGTA ATGCTATGGC TCTACTTCCA AGTAAGGGCG |
| 7321 | AATTCTGCAG ATATCCATCA CACTGGCGGC CGCTCGAGCA TGCATCTAGT GATATCCCTG |
| 7381 | TGTGAAATTG TTATCCGCTA CGCGTGATCG TTCAAACATT TGGCAATAAA GTTTCTTAAG |
| 7441 | ATTGAATCCT GTTGCCGGTC TTGCGATGAT TATCATATAA TTTCTGTTGA ATTACGTTAA |
| 7501 | GCATGTAATA ATTAACATGT AATGCATGAC GTTATTTATG AGATGGGTTT TTATGATTAG |
| 7561 | AGTCCCGCAA TTATACATTT AATACGCGAT AGAAAACAAA ATATAGCGCG CAAACTAGGA |
| 7621 | TAAATTATCG CGCGCGGTGT CATCTATGTT ACTAGATCCC ATGGGAAGTT CCTATTCCGA |
| 7681 | AGTTCCTATT CTCTGAAAAG TATAGGAACT TCAGCGATCG CAGACGTCGG GATCTTCTGC |
| 7741 | AAGCATCTCT ATTTCCTGAA GGTCTAACCT CGAAGATTTA AGATTTAATT ACGTTTATAA |
| 7801 | TTACAAAATT GATTCTAGTA TCTTTAATTT AATGCTTATA CATTATTAAT TAATTTAGTA |
| 7861 | CTTTCAATTT GTTTTCAGAA ATTATTTTAC TATTTTTTAT AAAATAAAAG GGAGAAAATG |
| 7921 | GCTATTTAAA TACTAGCCTA TTTTATTTCA ATTTTAGCTT AAAATCAGCC CCAATTAGCC |
| 7981 | CCAATTTCAA ATTCAAATGG TCCAGCCCAA TTCCTAAATA ACCCACCCCT AACCCGCCCG |
| 8041 | GTTTCCCCTT TTGATCCATG CAGTCAACGC CCAGAATTTC CCTATATAAT TTTTTAATTC |
| 8101 | CCAAACACCC CTAACTCTAT CCCATTTCTC ACCAACCGCC ACATAGATCT ATCCTCTTAT |
| 8161 | CTCTCAAACT CTCTCGAACC TTCCCCTAAC CCTAGCAGCC TCTCATCATC CTCACCTCAA |
| 8221 | AACCCACCGG GGCCGGCCAT GATTGAACAA GATGGATTGC ACGCAGGTTC TCCGGCCGCT |
| 8281 | TGGGTGGAGA GGCTATTCGG CTATGACTGG GCACAACAGA CAATCGGCTG CTCTGATGCC |
| 8341 | GCCGTGTTCC GGCTGTCAGC GCAGGGGAGG CCGGTTCTTT TTGTCAAGAC CGACCTGTCC |
| 8401 | GGTGCCCTGA ATGAACTTCA AGACGAGGCA GCGCGGCTAT CGTGGCTGGC CACGACGGGC |
| 8461 | GTTCCTTGCG CAGCTGTGCT CGACGTTGTC ACTGAAGCGG GAAGGGACTG GCTGCTATTG |
| 8521 | GGCGAAGTGC CGGGGCAGGA TCTCCTGTCA TCTCACCTTG CTCCTGCCGA GAAAGTATCC |
| 8581 | ATCATGGCTG ATGCAATGCG GCGGCTGCAT ACGCTTGATC CGGCTACCTG CCCATTCGAC |
| 8641 | CACCAAGCGA AACATCGCAT CGAGCGAGCA CGTACTCGGA TGGAAGCCGG TCTTGTCGAT |
| 8701 | CAGGATGATC TGGACGAAGA GCATCAGGGG CTCGCGCCAG CCGAACTGTT CGCCAGGCTC |
| 8761 | AAGGCGCGCA TCCCCGACGG CGAGGATCTC GTCGTGACTC ATGGCGATGC CTGCTTGCCG |
| 8821 | AATATCATGG TGGAAAATGG CCGCTTTTCT GGATTCATCG ACTGTGGCCG GCTGGGTGTG |
| 8881 | GCGGACCGCT ATCAGGACAT AGCGTTGGCT ACCCGTGATA TTGCTGAAGA GCTTGGCGGC |
| 8941 | GAATGGGCTG ACCGCTTCCT CGTGCTTTAC GGTATCGCCG CTCCCGATTC GCAGCGCATC |
| 9001 | GCCTTCTATC GCCTTCTTGA CGAGTTCTTC TGAGGCGCGC C |
| pORE_R2 35S-StAKIP3, 8813 bases | |
| SEQ ID No. 16 | |
| 1 | GATCGTTCAA ACATTTGGCA ATAAAGTTTC TTAAGATTGA ATCCTGTTGC CGGTCTTGCG |
| 61 | ATGATTATCA TATAATTTCT GTTGAATTAC GTTAAGCATG TAATAATTAA CATGTAATGC |
| 121 | ATGACGTTAT TTATGAGATG GGTTTTTATG ATTAGAGTCC CGCAATTATA CATTTAATAC |
| 181 | GCGATAGAAA ACAAAATATA GCGCGCAAAC TAGGATAAAT TATCGCGCGC GGTGTCATCT |
| 241 | ATGTTACTAG ATCCCTAGGG AAGTTCCTAT TCCGAAGTTC CTATTCTCTG AAAAGTATAG |
| 301 | GAACTTCTTT GCGTATTGGG CGCTCTTGGC CTTTTTGGCC ACCGGTCGTA CGGTTAAAAC |
| 361 | CACCCCAGTA CATTAAAAAC GTCCGCAATG TGTTATTAAG TTGTCTAAGC GTCAATTTGT |
| 421 | TTACACCACA ATATATCCTG CCACCAGCCA GCCAACAGCT CCCCGACCGG CAGCTCGGCA |
| 481 | CAAAATCACC ACTCGATACA GGCAGCCCAT CAGTCCACTA GACGCTCACC GGGCTGGTTG |
| 541 | CCCTCGCCGC TGGGCTGGCG GCCGTCTATG GCCCTGCAAA CGCGCCAGAA ACGCCGTCGA |
| 601 | AGCCGTGTGC GAGACACCGC AGCCGCCGGC GTTGTGGATA CCTCGCGGAA AACTTGGCCC |
| 661 | TCACTGACAG ATGAGGGGCG GACGTTGACA CTTGAGGGGC CGACTCACCC GGCGCGGCGT |
| 721 | TGACAGATGA GGGGCAGGCT CGATTTCGGC CGGCGACGTG GAGCTGGCCA GCCTCGCAAA |
| 781 | TCGGCGAAAA CGCCTGATTT TACGCGAGTT TCCCACAGAT GATGTGGACA AGCCTGGGGA |
| 841 | TAAGTGCCCT GCGGTATTGA CACTTGAGGG GCGCGACTAC TGACAGATGA GGGGCGCGAT |
| 901 | CCTTGACACT TGAGGGGCAG AGTGCTGACA GATGAGGGGC GCACCTATTG ACATTTGAGG |
| 961 | GGCTGTCCAC AGGCAGAAAA TCCAGCATTT GCAAGGGTTT CCGCCCGTTT TTCGGCCACC |
| 1021 | GCTAACCTGT CTTTTAACCT GCTTTTAAAC CAATATTTAT AAACCTTGTT TTTAACCAGG |
| 1081 | GCTGCGCCCT GTGCGCGTGA CCGCGCACGC CGAAGGGGGG TGCCCCCCCT TCTCGAACCC |
| 1141 | TCCCGGCCCG CTCTCGCGTT GGCAGCATCA CCCATAATTG TGGTTTCAAA ATCGGCTCCG |
| 1201 | TCGATACTAT GTTATACGCC AACTTTGAAA ACAACTTTGA AAAAGCTGTT TTCTGGTATT |
| 1261 | TAAGGTTTTA GAATGCAAGG AACAGTGAAT TGGAGTTCGT CTTGTTATAA TTAGCTTCTT |
| 1321 | GGGGTATCTT TAAATACTGT AGAAAAGAGG AAGGAAATAA TAAATGGCTA AAATGAGAAT |
| 1381 | ATCACCGGAA TTGAAAAAAC TGATCGAAAA ATACCGCTGC GTAAAAGATA CGGAAGGAAT |
| 1441 | GTCTCCTGCT AAGGTATATA AGCTGGTGGG AGAAAATGAA AACCTATATT TAAAAATGAC |
| 1501 | GGACAGCCGG TATAAAGGGA CCACCTATGA TGTGGAACGG GAAAAGGACA TGATGCTATG |
| 1561 | GCTGGAAGGA AAGCTGCCTG TTCCAAAGGT CCTGCACTTT GAACGGCATG ATGGCTGGAG |
| 1621 | CAATCTGCTC ATGAGTGAGG CCGATGGCGT CCTTTGCTCG GAAGAGTATG AAGATGAACA |
| 1681 | AAGCCCTGAA AAGATTATCG AGCTGTATGC GGAGTGCATC AGGCTCTTTC ACTCCATCGA |
| 1741 | CATATCGGAT TGTCCCTATA CGAATAGCTT AGACAGCCGC TTAGCCGAAT TGGATTACTT |
| 1801 | ACTGAATAAC GATCTGGCCG ATGTGGATTG CGAAAACTGG GAAGAAGACA CTCCATTTAA |
| 1861 | AGATCCGCGC GAGCTGTATG ATTTTTTAAA GACGGAAAAG CCCGAAGAGG AACTTGTCTT |
| 1921 | TTCCCACGGC GACCTGGGAG ACAGCAACAT CTTTGTGAAA GATGGCAAAG TAAGTGGCTT |
| 1981 | TATTGATCTT GGGAGAAGCG GCAGGGCGGA CAAGTGGTAT GACATTGCCT TCTGCGTCCG |
| 2041 | GTCGATCAGG GAGGATATTG GGGAAGAACA GTATGTCGAG CTATTTTTTG ACTTACTGGG |
| 2101 | GATCAAGCCT GATTGGGAGA AAATAAAATA TTATATTTTA CTGGATGAAT TGTTTTAGTA |
| 2161 | CCTAGATGTG GCGCAACGAT GCCGGCGACA AGCAGGAGCG CACCGACTTC TTCCGCATCA |
| 2221 | AGTGTTTTGG CTCTCAGGCC GAGGCCCACG GCAAGTATTT GGGCAAGGGG TCGCTGGTAT |
| 2281 | TCGTGCAGGG CAAGATTCGG AATACCAAGT ACGAGAAGGA CGGCCAGACG GTCTACGGGA |
| 2341 | CCGACTTCAT TGCCGATAAG GTGGATTATC TGGACACCAA GGCACCAGGC GGGTCAAATC |
| 2401 | AGGAATAAGG GCACATTGCC CCGGCGTGAG TCGGGGCAAT CCCGCAAGGA GGGTGAATGA |
| 2461 | ATCGGACGTT TGACCGGAAG GCATACAGGC AAGAACTGAT CGACGCGGGG TTTTCCGCCG |
| 2521 | AGGATGCCGA AACCATCGCA AGCCGCACCG TCATGCGTGC GCCCCGCGAA ACCTTCCAGT |
| 2581 | CCGTCGGCTC GATGGTCCAG CAAGCTACGG CCAAGATCGA GCGCGACAGC GTGCAACTGG |
| 2641 | CTCCCCCTGC CCTGCCCGCG CCATCGGCCG CCGTGGAGCG TTCGCGTCGT CTCGAACAGG |
| 2701 | AGGCGGCAGG TTTGGCGAAG TCGATGACCA TCGACACGCG AGGAACTATG ACGACCAAGA |
| 2761 | AGCGAAAAAC CGCCGGCGAG GACCTGGCAA AACAGGTCAG CGAGGCCAAG CAAGCCGCGT |
| 2821 | TGCTGAAACA CACGAAGCAG CAGATCAAGG AAATGCAGCT TTCCTTGTTC GATATTGCGC |
| 2881 | CGTGGCCGGA CACGATGCGA GCGATGCCAA ACGACACGGC CCGCTCTGCC CTGTTCACCA |
| 2941 | CGCGCAACAA GAAAATCCCG CGCGAGGCGC TGCAAAACAA GGTCATTTTC CACGTCAACA |
| 3001 | AGGACGTGAA GATCACCTAC ACCGGCGTCG AGCTGCGGGC CGACGATGAC GAACTGGTGT |
| 3061 | GGCAGCAGGT GTTGGAGTAC GCGAAGCGCA CCCCTATCGG CGAGCCGATC ACCTTCACGT |
| 3121 | TCTACGAGCT TTGCCAGGAC CTGGGCTGGT CGATCAATGG CCGGTATTAC ACGAAGGCCG |
| 3181 | AGGAATGCCT GTCGCGCCTA CAGGCGACGG CGATGGGCTT CACGTCCGAC CGCGTTGGGC |
| 3241 | ACCTGGAATC GGTGTCGCTG CTGCACCGCT TCCGCGTCCT GGACCGTGGC AAGAAAACGT |
| 3301 | CCCGTTGCCA GGTCCTGATC GACGAGGAAA TCGTCGTGCT GTTTGCTGGC GACCACTACA |
| 3361 | CGAAATTCAT ATGGGAGAAG TACCGCAAGC TGTCGCCGAC GGCCCGACGG ATGTTCGACT |
| 3421 | ATTTCAGCTC GCACCGGGAG CCGTACCCGC TCAAGCTGGA AACCTTCCGC CTCATGTGCG |
| 3481 | GATCGGATTC CACCCGCGTG AAGAAGTGGC GCGAGCAGGT CGGCGAAGCC TGCGAAGAGT |
| 3541 | TGCGAGGCAG CGGCCTGGTG GAACACGCCT GGGTCAATGA TGACCTGGTG CATTGCAAAC |
| 3601 | GCTAGGGCCT TGTGGGGTCA GTTCCGGCTG GGGGTTCAGC AGCCAGCGCT TTACTGAGAT |
| 3661 | CCTCTTCCGC TTCCTCGCTC ACTGACTCGC TGCGCTCGGT CGTTCGGCTG CGGCGAGCGG |
| 3721 | TATCAGCTCA CTCAAAGGCG GTAATACGGT TATCCACAGA ATCAGGGGAT AACGCAGGAA |
| 3781 | AGAACATGTG AGCAAAAGGC CAGCAAAAGG CCAGGAACCG TAAAAAGGCC GCGTTGCTGG |
| 3841 | CGTTTTTCCA TAGGCTCCGC CCCCCTGACG AGCATCACAA AAATCGACGC TCAAGTCAGA |
| 3901 | GGTGGCGAAA CCCGACAGGA CTATAAAGAT ACCAGGCGTT TCCCCCTGGA AGCTCCCTCG |
| 3961 | TGCGCTCTCC TGTTCCGACC CTGCCGCTTA CCGGATACCT GTCCGCCTTT CTCCCTTCGG |
| 4021 | GAAGCGTGGC GCTTTCTCAT AGCTCACGCT GTAGGTATCT CAGTTCGGTG TAGGTCGTTC |
| 4081 | GCTCCAAGCT GGGCTGTGTG CACGAACCCC CCGTTCAGCC CGACCGCTGC GCCTTATCCG |
| 4141 | GTAACTATCG TCTTGAGTCC AACCCGGTAA GACACGACTT ATCGCCACTG GCAGCAGCCA |
| 4201 | CTGGTAACAG GATTAGCAGA GCGAGGTATG TAGGCGGTGC TACAGAGTTC TTGAAGTGGT |
| 4261 | GGCCTAACTA CGGCTACACT AGAAGAACAG TATTTGGTAT CTGCGCTCTG CTGAAGCCAG |
| 4321 | TTACCTTCGG AAAAAGAGTT GGTAGCTCTT GATCCGGCAA ACAAACCACC GCTGGTAGCG |
| 4381 | GTGGTTTTTT TGTTTGCAAG CAGCAGATTA CGCGCAGAAA AAAAGGATCT CAAGAAGATC |
| 4441 | CTTTGATCTT TTCTACGGGG TCTGACGCTC AGTGGAACGA AAACTCACGT TAAGGGATTT |
| 4501 | TGGTCATGAG ATTATCAAAA AGGATCTTCA CCTAGATCCT TTTGGATCTC CTGTGGTTGG |
| 4561 | CATGCACATA CAAATGGACG AACGGATAAA CCTTTTCACG CCCTTTTAAA TATCCGATTA |
| 4621 | TTCTAATAAA CGCTCTTTTC TCTTAGGTTT ACCCGCCAAT ATATCCTGTC AAACACTGAT |
| 4681 | AGTTTAAACT GAAGGCGGGA AACGACAATC TGCTAGTGGA TCTCCCAGTC ACGACGTTGT |
| 4741 | AAAACGGGCG CCCCGCGGAA AGCTTGCATG CCTGCAGGTC CCCAGATTAG CCTTTTCAAT |
| 4801 | TTCAGAAAGA ATGCTAACCC ACAGATGGTT AGAGAGGCTT ACGCAGCAGG TCTCATCAAG |
| 4861 | ACGATCTACC CGAGCAATAA TCTCCAGGAA ATCAAATACC TTCCCAAGAA GGTTAAAGAT |
| 4921 | GCAGTCAAAA GATTCAGGAC TAACTGCATC AAGAACACAG AGAAAGATAT ATTTCTCAAG |
| 4981 | ATCAGAAGTA CTATTCCAGT ATGGACGATT CAAGGCTTGC TTCACAAACC AAGGCAAGTA |
| 5041 | ATAGAGATTG GAGTCTCTAA AAAGGTAGTT CCCACTGAAT CAAAGGCCAT GGAGTCAAAG |
| 5101 | ATTCAAATAG AGGACCTAAC AGAACTCGCC GTAAAGACTG GCGAACAGTT CATACAGAGT |
| 5161 | CTCTTACGAC TCAATGACAA GAAGAAAATC TTCGTCAACA TGGTGGAGCA CGACACACTT |
| 5221 | GTCTACTCCA AAAATATCAA AGATACAGTC TCAGAAGACC AAAGGGCAAT TGAGACTTTT |
| 5281 | CAACAAAGGG TAATATCCGG AAACCTCCTC GGATTCCATT GCCCAGCTAT CTGTCACTTT |
| 5341 | ATTGTGAAGA TAGTGGAAAA GGAAGGTGGC TCCTACAAAT GCCATCATTG CGATAAAGGA |
| 5401 | AAGGCCATCG TTGAAGATGC CTCTGCCGAC AGTGGTCCCA AAGATGGACC CCCACCCACG |
| 5461 | AGGAGCATCG TGGAAAAAGA AGACGTTCCA ACCACGTCTT CAAAGCAAGT GGATTGATGT |
| 5521 | GATATCTCCA CTGACGTAAG GGATGACGCA CAATCCCACT ATCCTTCGCA AGACCCTTCC |
| 5581 | TCTATATAAG GAAGTTCATT TCATTTGGAG AGAACACGGG GGACTCTAGA AGGCCTTGGA |
| 5641 | TCCACCCGGG AGAATTCGTC GACTTTGCGG CCGCATCGAT ACTGCAGGAG CTCGGATCCA |
| 5701 | CTAGTAACGG CCGCCAGTGT GCTGGAATTC GCCCTTTCTT CTCTGAGCGA AAATGGATCT |
| 5761 | AACAAAGAAA CGCAAAGCTG ATGAGAACGG CGGTGCTTAT CCCGTATCCA ACGAACTCGT |
| 5821 | CACAACTGCC GCCGCGCCGC CGGCACTTGC TGTTCTTTCC CCGGAAGACA TCGACAAAAT |
| 5881 | TCTTGAAACA TTCACCAAAG ACCAGTGCGT TTCAATTCTC CGTAACGCCG CCCTACGCTA |
| 5941 | TGCTGATGTT CTCGAAGGTC TACGTGCTGT CGCTGACGCC GACGTATCTA ACCGCAAGCT |
| 6001 | CTTCGTTCGT GGTCTTGGTT GGGAGACCAC CACCGACAAA CTCCGACAGG TTTTTTCTGA |
| 6061 | GTTTGGAGAA CTCGATGAGG CTGTAGTTAT AACTGATAAG GCGTCTTCCA GATCTAAAGG |
| 6121 | GTATGGTTTT GTAACTTTCA AGCACGTTGA TGCTGCTATT CTTTCTTTAA AGGTGCCCAA |
| 6181 | CAAGATAATT GATGGCCGTG TTACCGTCAC ACAGCTTGCT GCTGCGGGTA ATTCTGGGAA |
| 6241 | TTCTCAGTCT TCTGATGTTG CGCTTAGGAA GATCTATGTT GGTAATGTTC CATTTGAAAT |
| 6301 | TTCGTCTGAG AAGCTTTTGA ATCACTTTTC TATGTATGGA GAGATTGAAG AGGGGCCTTT |
| 6361 | GGGATTTGAT AAACAAACTG GAAAAGCTAA AGGTTTTGCT TTTTTTGTCT ACAAGACTGA |
| 6421 | GGACGGAGCT AGGGCATCTT TAGTTGATCC CGTGAAGACA GTAGAAGGAC ATCAGGTCTT |
| 6481 | GTGTAAATTG GCAACCGATA ATAAGAAGGG GAAGCCCCAG AATATGGGGC ATGGGGGTGC |
| 6541 | CCCTGGAATG AATGCTGGAC CTGGAGGAAT GCCTGGTGAT GATAGGGTTG GATCCATGCC |
| 6601 | CGGTTCAAAT TATGGTGTTC CTGTTAGCGG TTTGGGTCCA TATTCAGGGT TTTCAGGTGG |
| 6661 | GCCTGGTCCA GGAATGCAGC AGCCACCACC TCAGCCTGGC ATGGTGGCAC ATCAGAATCC |
| 6721 | ACATCTGAAT TCAGCTATTG GCGGGCCTGG ATATGGAAAC CAAGGACCAG GATCCTTTAC |
| 6781 | AGGTGGTGCT GGTGGATATG CTGGCGCTGG TGGATATGCT GGGGCTGGTG GGTATGGTGG |
| 6841 | TAGTTCTGGG GATTACAGTG GGTACAGGAT GCCTCAAAGC TCTGCTGGAA TGCCTAGTTC |
| 6901 | AGGAGGTTAT CCAGATGGTG GTAATTATGG TTTGCAGTCT TCTTATCCCT CACAGGTACC |
| 6961 | ACAACCAGGA GCTGGGTCAA GGGTTCCACC AGGTGGGATG TACCAGGGCA TGCCTCCATA |
| 7021 | CTACTGAGGT GTCTTGAGAG CTTCTTGGAT GCAACATGCT TTAAATGGTT GTGGTGATTT |
| 7081 | CCTGAAAGGG CGAATTCTGC AGATATCCAT CACACTGGCG GCCGCTCGAG CATGCATCTA |
| 7141 | GTGATATCCC TGTGTGAAAT TGTTATCCGC TACGCGTGAT CGTTCAAACA TTTGGCAATA |
| 7201 | AAGTTTCTTA AGATTGAATC CTGTTGCCGG TCTTGCGATG ATTATCATAT AATTTCTGTT |
| 7261 | GAATTACGTT AAGCATGTAA TAATTAACAT GTAATGCATG ACGTTATTTA TGAGATGGGT |
| 7321 | TTTTATGATT AGAGTCCCGC AATTATACAT TTAATACGCG ATAGAAAACA AAATATAGCG |
| 7381 | CGCAAACTAG GATAAATTAT CGCGCGCGGT GTCATCTATG TTACTAGATC CCATGGGAAG |
| 7441 | TTCCTATTCC GAAGTTCCTA TTCTCTGAAA AGTATAGGAA CTTCAGCGAT CGCAGACGTC |
| 7501 | GGGATCTTCT GCAAGCATCT CTATTTCCTG AAGGTCTAAC CTCGAAGATT TAAGATTTAA |
| 7561 | TTACGTTTAT AATTACAAAA TTGATTCTAG TATCTTTAAT TTAATGCTTA TACATTATTA |
| 7621 | ATTAATTTAG TACTTTCAAT TTGTTTTCAG AAATTATTTT ACTATTTTTT ATAAAATAAA |
| 7681 | AGGGAGAAAA TGGCTATTTA AATACTAGCC TATTTTATTT CAATTTTAGC TTAAAATCAG |
| 7741 | CCCCAATTAG CCCCAATTTC AAATTCAAAT GGTCCAGCCC AATTCCTAAA TAACCCACCC |
| 7801 | CTAACCCGCC CGGTTTCCCC TTTTGATCCA TGCAGTCAAC GCCCAGAATT TCCCTATATA |
| 7861 | ATTTTTTAAT TCCCAAACAC CCCTAACTCT ATCCCATTTC TCACCAACCG CCACATAGAT |
| 7921 | CTATCCTCTT ATCTCTCAAA CTCTCTCGAA CCTTCCCCTA ACCCTAGCAG CCTCTCATCA |
| 7981 | TCCTCACCTC AAAACCCACC GGGGCCGGCC ATGATTGAAC AAGATGGATT GCACGCAGGT |
| 8041 | TCTCCGGCCG CTTGGGTGGA GAGGCTATTC GGCTATGACT GGGCACAACA GACAATCGGC |
| 8101 | TGCTCTGATG CCGCCGTGTT CCGGCTGTCA GCGCAGGGGA GGCCGGTTCT TTTTGTCAAG |
| 8161 | ACCGACCTGT CCGGTGCCCT GAATGAACTT CAAGACGAGG CAGCGCGGCT ATCGTGGCTG |
| 8221 | GCCACGACGG GCGTTCCTTG CGCAGCTGTG CTCGACGTTG TCACTGAAGC GGGAAGGGAC |
| 8281 | TGGCTGCTAT TGGGCGAAGT GCCGGGGCAG GATCTCCTGT CATCTCACCT TGCTCCTGCC |
| 8341 | GAGAAAGTAT CCATCATGGC TGATGCAATG CGGCGGCTGC ATACGCTTGA TCCGGCTACC |
| 8401 | TGCCCATTCG ACCACCAAGC GAAACATCGC ATCGAGCGAG CACGTACTCG GATGGAAGCC |
| 8461 | GGTCTTGTCG ATCAGGATGA TCTGGACGAA GAGCATCAGG GGCTCGCGCC AGCCGAACTG |
| 8521 | TTCGCCAGGC TCAAGGCGCG CATGCCCGAC GGCGAGGATC TCGTCGTGAC TCATGGCGAT |
| 8581 | GCCTGCTTGC CGAATATCAT GGTGGAAAAT GGCCGCTTTT CTGGATTCAT CGACTGTGGC |
| 8641 | CGGCTGGGTG TGGCGGACCG CTATCAGGAC ATAGCGTTGG CTACCCGTGA TATTGCTGAA |
| 8701 | GAGCTTGGCG GCGAATGGGC TGACCGCTTC CTCGTGCTTT ACGGTATCGC CGCTCCCGAT |
| 8761 | TCGCAGCGCA TCGCCTTCTA TCGCCTTCTT GACGAGTTCT TCTGAGGCGC GCC |
| pORE_R2 pSAG12:Flag-Akip2, 10130 bases | |
| SEQ ID No. 17 | |
| 1 | GATCGTTCAA ACATTTGGCA ATAAAGTTTC TTAAGATTGA ATCCTGTTGC CGGTCTTGCG |
| 61 | ATGATTATCA TATAATTTCT GTTGAATTAC GTTAAGCATG TAATAATTAA CATGTAATGC |
| 121 | ATGACGTTAT TTATGAGATG GGTTTTTATG ATTAGAGTCC CGCAATTATA CATTTAATAC |
| 181 | GCGATAGAAA ACAAAATATA GCGCGCAAAC TAGGATAAAT TATCGCGCGC GGTGTCATCT |
| 241 | ATGTTACTAG ATCCCTAGGG AAGTTCCTAT TCCGAAGTTC CTATTCTCTG AAAAGTATAG |
| 301 | GAACTTCTTT GCGTATTGGG CGCTCTTGGC CTTTTTGGCC ACCGGTCGTA CGGTTAAAAC |
| 361 | CACCCCAGTA CATTAAAAAC GTCCGCAATG TGTTATTAAG TTGTCTAAGC GTCAATTTGT |
| 421 | TTACACCACA ATATATCCTG CCACCAGCCA GCCAACAGCT CCCCGACCGG CAGCTCGGCA |
| 481 | CAAAATCACC ACTCGATACA GGCAGCCCAT CAGTCCACTA GACGCTCACC GGGCTGGTTG |
| 541 | CCCTCGCCGC TGGGCTGGCG GCCGTCTATG GCCCTGCAAA CGCGCCAGAA ACGCCGTCGA |
| 601 | AGCCGTGTGC GAGACACCGC AGCCGCCGGC GTTGTGGATA CCTCGCGGAA AACTTGGCCC |
| 661 | TCACTGACAG ATGAGGGGCG GACGTTGACA CTTGAGGGGC CGACTCACCC GGCGCGGCGT |
| 721 | TGACAGATGA GGGGCAGGCT CGATTTCGGC CGGCGACGTG GAGCTGGCCA GCCTCGCAAA |
| 781 | TCGGCGAAAA CGCCTGATTT TACGCGAGTT TCCCACAGAT GATGTGGACA AGCCTGGGGA |
| 841 | TAAGTGCCCT GCGGTATTGA CACTTGAGGG GCGCGACTAC TGACAGATGA GGGGCGCGAT |
| 901 | CCTTGACACT TGAGGGGCAG AGTGCTGACA GATGAGGGGC GCACCTATTG ACATTTGAGG |
| 961 | GGCTGTCCAC AGGCAGAAAA TCCAGCATTT GCAAGGGTTT CCGCCCGTTT TTCGGCCACC |
| 1021 | GCTAACCTGT CTTTTAACCT GCTTTTAAAC CAATATTTAT AAACCTTGTT TTTAACCAGG |
| 1081 | GCTGCGCCCT GTGCGCGTGA CCGCGCACGC CGAAGGGGGG TGCCCCCCCT TCTCGAACCC |
| 1141 | TCCCGGCCCG CTCTCGCGTT GGCAGCATCA CCCATAATTG TGGTTTCAAA ATCGGCTCCG |
| 1201 | TCGATACTAT GTTATACGCC AACTTTGAAA ACAACTTTGA AAAAGCTGTT TTCTGGTATT |
| 1261 | TAAGGTTTTA GAATGCAAGG AACAGTGAAT TGGAGTTCGT CTTGTTATAA TTAGCTTCTT |
| 1321 | GGGGTATCTT TAAATACTGT AGAAAAGAGG AAGGAAATAA TAAATGGCTA AAATGAGAAT |
| 1381 | ATCACCGGAA TTGAAAAAAC TGATCGAAAA ATACCGCTGC GTAAAAGATA CGGAAGGAAT |
| 1441 | GTCTCCTGCT AAGGTATATA AGCTGGTGGG AGAAAATGAA AACCTATATT TAAAAATGAC |
| 1501 | GGACAGCCGG TATAAAGGGA CCACCTATGA TGTGGAACGG GAAAAGGACA TGATGCTATG |
| 1561 | GCTGGAAGGA AAGCTGCCTG TTCCAAAGGT CCTGCACTTT GAACGGCATG ATGGCTGGAG |
| 1621 | CAATCTGCTC ATGAGTGAGG CCGATGGCGT CCTTTGCTCG GAAGAGTATG AAGATGAACA |
| 1681 | AAGCCCTGAA AAGATTATCG AGCTGTATGC GGAGTGCATC AGGCTCTTTC ACTCCATCGA |
| 1741 | CATATCGGAT TGTCCCTATA CGAATAGCTT AGACAGCCGC TTAGCCGAAT TGGATTACTT |
| 1801 | ACTGAATAAC GATCTGGCCG ATGTGGATTG CGAAAACTGG GAAGAAGACA CTCCATTTAA |
| 1861 | AGATCCGCGC GAGCTGTATG ATTTTTTAAA GACGGAAAAG CCCGAAGAGG AACTTGTCTT |
| 1921 | TTCCCACGGC GACCTGGGAG ACAGCAACAT CTTTGTGAAA GATGGCAAAG TAAGTGGCTT |
| 1981 | TATTGATCTT GGGAGAAGCG GCAGGGCGGA CAAGTGGTAT GACATTGCCT TCTGCGTCCG |
| 2041 | GTCGATCAGG GAGGATATTG GGGAAGAACA GTATGTCGAG CTATTTTTTG ACTTACTGGG |
| 2101 | GATCAAGCCT GATTGGGAGA AAATAAAATA TTATATTTTA CTGGATGAAT TGTTTTAGTA |
| 2161 | CCTAGATGTG GCGCAACGAT GCCGGCGACA AGCAGGAGCG CACCGACTTC TTCCGCATCA |
| 2221 | AGTGTTTTGG CTCTCAGGCC GAGGCCCACG GCAAGTATTT GGGCAAGGGG TCGCTGGTAT |
| 2281 | TCGTGCAGGG CAAGATTCGG AATACCAAGT ACGAGAAGGA CGGCCAGACG GTCTACGGGA |
| 2341 | CCGACTTCAT TGCCGATAAG GTGGATTATC TGGACACCAA GGCACCAGGC GGGTCAAATC |
| 2401 | AGGAATAAGG GCACATTGCC CCGGCGTGAG TCGGGGCAAT CCCGCAAGGA GGGTGAATGA |
| 2461 | ATCGGACGTT TGACCGGAAG GCATACAGGC AAGAACTGAT CGACGCGGGG TTTTCCGCCG |
| 2521 | AGGATGCCGA AACCATCGCA AGCCGCACCG TCATGCGTGC GCCCCGCGAA ACCTTCCAGT |
| 2581 | CCGTCGGCTC GATGGTCCAG CAAGCTACGG CCAAGATCGA GCGCGACAGC GTGCAACTGG |
| 2641 | CTCCCCCTGC CCTGCCCGCG CCATCGGCCG CCGTGGAGCG TTCGCGTCGT CTCGAACAGG |
| 2701 | AGGCGGCAGG TTTGGCGAAG TCGATGACCA TCGACACGCG AGGAACTATG ACGACCAAGA |
| 2761 | AGCGAAAAAC CGCCGGCGAG GACCTGGCAA AACAGGTCAG CGAGGCCAAG CAAGCCGCGT |
| 2821 | TGCTGAAACA CACGAAGCAG CAGATCAAGG AAATGCAGCT TTCCTTGTTC GATATTGCGC |
| 2881 | CGTGGCCGGA CACGATGCGA GCGATGCCAA ACGACACGGC CCGCTCTGCC CTGTTCACCA |
| 2941 | CGCGCAACAA GAAAATCCCG CGCGAGGCGC TGCAAAACAA GGTCATTTTC CACGTCAACA |
| 3001 | AGGACGTGAA GATCACCTAC ACCGGCGTCG AGCTGCGGGC CGACGATGAC GAACTGGTGT |
| 3061 | GGCAGCAGGT GTTGGAGTAC GCGAAGCGCA CCCCTATCGG CGAGCCGATC ACCTTCACGT |
| 3121 | TCTACGAGCT TTGCCAGGAC CTGGGCTGGT CGATCAATGG CCGGTATTAC ACGAAGGCCG |
| 3181 | AGGAATGCCT GTCGCGCCTA CAGGCGACGG CGATGGGCTT CACGTCCGAC CGCGTTGGGC |
| 3241 | ACCTGGAATC GGTGTCGCTG CTGCACCGCT TCCGCGTCCT GGACCGTGGC AAGAAAACGT |
| 3301 | CCCGTTGCCA GGTCCTGATC GACGAGGAAA TCGTCGTGCT GTTTGCTGGC GACCACTACA |
| 3361 | CGAAATTCAT ATGGGAGAAG TACCGCAAGC TGTCGCCGAC GGCCCGACGG ATGTTCGACT |
| 3421 | ATTTCAGCTC GCACCGGGAG CCGTACCCGC TCAAGCTGGA AACCTTCCGC CTCATGTGCG |
| 3481 | GATCGGATTC CACCCGCGTG AAGAAGTGGC GCGAGCAGGT CGGCGAAGCC TGCGAAGAGT |
| 3541 | TGCGAGGCAG CGGCCTGGTG GAACACGCCT GGGTCAATGA TGACCTGGTG CATTGCAAAC |
| 3601 | GCTAGGGCCT TGTGGGGTCA GTTCCGGCTG GGGGTTCAGC AGCCAGCGCT TTACTGAGAT |
| 3661 | CCTCTTCCGC TTCCTCGCTC ACTGACTCGC TGCGCTCGGT CGTTCGGCTG CGGCGAGCGG |
| 3721 | TATCAGCTCA CTCAAAGGCG GTAATACGGT TATCCACAGA ATCAGGGGAT AACGCAGGAA |
| 3781 | AGAACATGTG AGCAAAAGGC CAGCAAAAGG CCAGGAACCG TAAAAAGGCC GCGTTGCTGG |
| 3841 | CGTTTTTCCA TAGGCTCCGC CCCCCTGACG AGCATCACAA AAATCGACGC TCAAGTCAGA |
| 3901 | GGTGGCGAAA CCCGACAGGA CTATAAAGAT ACCAGGCGTT TCCCCCTGGA AGCTCCCTCG |
| 3961 | TGCGCTCTCC TGTTCCGACC CTGCCGCTTA CCGGATACCT GTCCGCCTTT CTCCCTTCGG |
| 4021 | GAAGCGTGGC GCTTTCTCAT AGCTCACGCT GTAGGTATCT CAGTTCGGTG TAGGTCGTTC |
| 4081 | GCTCCAAGCT GGGCTGTGTG CACGAACCCC CCGTTCAGCC CGACCGCTGC GCCTTATCCG |
| 4141 | GTAACTATCG TCTTGAGTCC AACCCGGTAA GACACGACTT ATCGCCACTG GCAGCAGCCA |
| 4201 | CTGGTAACAG GATTAGCAGA GCGAGGTATG TAGGCGGTGC TACAGAGTTC TTGAAGTGGT |
| 4261 | GGCCTAACTA CGGCTACACT AGAAGAACAG TATTTGGTAT CTGCGCTCTG CTGAAGCCAG |
| 4321 | TTACCTTCGG AAAAAGAGTT GGTAGCTCTT GATCCGGCAA ACAAACCACC GCTGGTAGCG |
| 4381 | GTGGTTTTTT TGTTTGCAAG CAGCAGATTA CGCGCAGAAA AAAAGGATCT CAAGAAGATC |
| 4441 | CTTTGATCTT TTCTACGGGG TCTGACGCTC AGTGGAACGA AAACTCACGT TAAGGGATTT |
| 4501 | TGGTCATGAG ATTATCAAAA AGGATCTTCA CCTAGATCCT TTTGGATCTC CTGTGGTTGG |
| 4561 | CATGCACATA CAAATGGACG AACGGATAAA CCTTTTCACG CCCTTTTAAA TATCCGATTA |
| 4621 | TTCTAATAAA CGCTCTTTTC TCTTAGGTTT ACCCGCCAAT ATATCCTGTC AAACACTGAT |
| 4681 | AGTTTAAACT GAAGGCGGGA AACGACAATC TGCTAGTGGA TCTCCCAGTC ACGACGTTGT |
| 4741 | AAAACGGGCG CCCCGCGGga tatctctttt tatattcaaa caataagttg agatatgttt |
| 4801 | gagaagagga caactattct cgtggagcac cgagtttgtt ttatattaga aacccgattg |
| 4861 | ttatttttag actgagacaa aaaagtaaaa tcgttgattg ttaaaattta aaattagttt |
| 4921 | cattacgttt cgataaaaaa atgattagtt tatcatagct taattatagc attgatttct |
| 4981 | aaatttgttt tttgaccacc cttttttctc tctttggtgt tttcttaaca ttagaagaac |
| 5041 | ccataacaat gtacgttcaa attaattaaa aacaatattt ccaagtttta tatacgaaac |
| 5101 | ttgttttttt taatgaaaac agttgaatag ttgattatga attagttaga tcaatactca |
| 5161 | atatatgatc aatgatgtat atatatgaac tcagttgtta tacaagaaat gaaaatgcta |
| 5221 | tttaaatacc gatcatgaag tgttaaaaag tgtcagaata tgacatgaag cgttttgtcc |
| 5281 | taccgggtat tcgagttata ggtttggatc tctcaagaat attttgggcc atattagtta |
| 5341 | tatttgggct taagcgtttt gcaaagagac gaggaagaaa gattgggtca agttaacaaa |
| 5401 | acagagacac tcgtattagt tggtactttg gtagcaagtc gatttatttg ccagtaaaaa |
| 5461 | cttggtacac aactgacaac tcgtatcgtt attagtttgt acttggtacc tttggttcaa |
| 5521 | gaaaaagttg atatagttaa atcagttgtg ttcatgaggt gattgtgatt taatttgttg |
| 5581 | actagggcga ttccttcaca tcacaataac aaagttttat agattttttt tttataacat |
| 5641 | ttttgccacg cttcgtaaag tttggtattt acaccgcatt tttccctgta caagaattca |
| 5701 | tatattattt atttatatac tccagttgac aattataagt ttataacgtt tttacaatta |
| 5761 | tttaaatacc atgtgaagat ccaagaatat gtcttacttc ttctttgtgt aagaaaacta |
| 5821 | actatatcac tataataaaa taattctaat cattatattt gtaaatatgc agttatttgt |
| 5881 | caattttgaa tttagtattt tagacgttat cacttcagcc aaatatgatt tggatttaag |
| 5941 | tccaaaatgc aatttcgtac gtatccctct tgtcgtctaa tgattatttc aatatttctt |
| 6001 | atattatccc taactacaga gctacattta tattgtattc taatgacagg gaaactttca |
| 6061 | tagagattca gatagatgaa attggtggga aacatcattg aacaggaaac ttttagcaaa |
| 6121 | tcatatcgat ttatctacaa aagaatactt agcgtaatga agttcacttg ttgtgaatga |
| 6181 | ctatgatttg atcaaattag ttaattttgt cgaatcattt ttctttttga tttgattaag |
| 6241 | cttttaactt gcacgaatgg ttctcttgtg aataaacaga atctttgaat tcaaactatt |
| 6301 | tgattagtga aaagacaaaa gaagattcct tgtttttatg tgattagtga ttttgatgca |
| 6361 | tgaaaggtac ctacgtacta caagaaaaat aaacatgtac gtaactacgt atcagcatgt |
| 6421 | aaaagtattt ttttccaaat aatttatact catgatagat tttttttttt tgaaatgtca |
| 6481 | attaaaaatg ctttcttaaa tattaatttt aattaattaa ataaggaaat atatttatgc |
| 6541 | aaaacatcat caacacatat ccaacttcga aaatctctat agtacacaag tagagaaatt |
| 6601 | aaattttact agatacaaac ttcctaatca tcaaatataa atgtttacaa aactaattaa |
| 6661 | acccaccact aaaattaact aaaaatccga gcaaagtgag tgaacaagac ttgatttcag |
| 6721 | gttgatgtag gactaaaatg gctacgtatc aaacatcaac gatcatttag ttatgtatga |
| 6781 | atgaatgtag tcattacttg taaaacaaaa atgctttgat ttggatcaat cacttcatgt |
| 6841 | gaacattagc aattacatca accttatttt cactataaaa ccccatctca gtacccttct |
| 6901 | gaagtaatca aattaagagc aaaagtcatt taactttcct aaaacaCTCG AGGTATTTTT |
| 6961 | ACAACAATTA CCAACAACAA CAAACAACAA ACAACATTAC AATTACTATT TACAATTACA |
| 7021 | ATTACCATGG ACTACAAGGA CGACGATGAC AAGCATATGA CAAAGAAGAG AAAGCTCGAA |
| 7081 | TCTGAATCCA ACGAAACGTC AGAGCCGACG GAGAAGCAGC AGCAGCAATG TGAAAAAGAG |
| 7141 | GATCCGGAAA TCAGAAATGT TGATAATCAA AGAGACGACG ACGAACAAGT AGTAGAGCAA |
| 7201 | GACACACTAA AGGAGATGCA CGAAGAGGAA GCTAAAGGTG AAGATAACAT AGAAGCGGAG |
| 7261 | ACGTCGTCCG GATCTGGGAA TCAAGGAAAT GAGGATGATG ACGAAGAAGA ACCTATTGAG |
| 7321 | GATCTATTGG AACCGTTTTC AAAGGATCAA CTTTTGATTC TTCTCAAGGA AGCTGCAGAG |
| 7381 | AGACATCGCG ATGTAGCTAA TCGAATCCGG ATTGTGGCGG ATGAAGATCT TGTTCATCGT |
| 7441 | AAGATCTTCG TTCACGGGCT TGGATGGGAT ACTAAAGCTG ATTCACTTAT CGACGCTTTT |
| 7501 | AAACAGTACG GAGAGATTGA AGATTGCAAA TGTGTGGTTG ATAAGGTATC TGGGCAATCT |
| 7561 | AAAGGTTATG GCTTTATCCT CTTTAAGTCA AGGTCTGGTG CTCGTAACGC TCTTAAGCAG |
| 7621 | CCTCAGAAGA AGATTGGAAC TCGTATGACT GCGTGCCAGC TGGCGTCTAT AGGACCTGTT |
| 7681 | CAGGGGAACC CTGTTGTGGC TCCTGCTCAG CATTTCAATC CTGAGAATGT TCAGAGGAAG |
| 7741 | ATTTATGTCA GTAACGTTAG TGCAGACATT GATCCGCAGA AGTTGCTGGA GTTTTTCTCA |
| 7801 | AGGTTTGGGG AGATAGAAGA AGGTCCTTTG GGGCTTGATA AAGCTACTGG GAGACCTAAA |
| 7861 | GGTTTCGCTT TGTTTGTCTA TAGATCCCTA GAAAGTGCCA AGAAGGCATT GGAGGAGCCA |
| 7921 | CACAAGACTT TTGAAGGCCA TGTCTTGCAT TGCCACAAAG CAAATGATGG GCCAAAACAG |
| 7981 | GTTAAGCAAC ATCAACATAA CCATAACTCT CACAATCAAA ATTCCCGTTA CCAAAGGAAC |
| 8041 | GACAACAATG GTTATGGTGC CCCTGGAGGC CATGGACATT TCATAGCTGG TAATAACCAA |
| 8101 | GCTGTGCAGG CGTTTAATCC GGCCATTGGC CAGGCCCTCA CAGCTTTGCT GGCATCTCAG |
| 8161 | GGTGCTGGGT TGGGTTTAAA CCAAGCATTT GGGCAGGCTT TGTTGGGGAC ATTAGGGACA |
| 8221 | GCTAGCCCAG GAGCTGTAGG TGGAATGCCA AGTGGCTATG GTACTCAAGC AAATATCTCA |
| 8281 | CCTGGGGTCT ATCCTGGGTA CGGTGCTCAA GCCGGGTACC AGGGCGGTTA TCAGACTCAG |
| 8341 | CAACCTGGTC AGGGCGGTGC GGGAAGAGGG CAGCATGGTG CTGGGTATGG TGGTCCTTAC |
| 8401 | ATGGGTCGTT AGATTAGCCA CTCAGGAAGG GCGAATTCGT TTAAACCTGC AGGACTAGTG |
| 8461 | ATATCCCTGT GTGAAATTGT TATCCGCTAC GCGTGATCGT TCAAACATTT GGCAATAAAG |
| 8521 | TTTCTTAAGA TTGAATCCTG TTGCCGGTCT TGCGATGATT ATCATATAAT TTCTGTTGAA |
| 8581 | TTACGTTAAG CATGTAATAA TTAACATGTA ATGCATGACG TTATTTATGA GATGGGTTTT |
| 8641 | TATGATTAGA GTCCCGCAAT TATACATTTA ATACGCGATA GAAAACAAAA TATAGCGCGC |
| 8701 | AAACTAGGAT AAATTATCGC GCGCGGTGTC ATCTATGTTA CTAGATCCCA TGGGAAGTTC |
| 8761 | CTATTCCGAA GTTCCTATTC TCTGAAAAGT ATAGGAACTT CAGCGATCGC AGACGTCGGG |
| 8821 | ATCTTCTGCA AGCATCTCTA TTTCCTGAAG GTCTAACCTC GAAGATTTAA GATTTAATTA |
| 8881 | CGTTTATAAT TACAAAATTG ATTCTAGTAT CTTTAATTTA ATGCTTATAC ATTATTAATT |
| 8941 | AATTTAGTAC TTTCAATTTG TTTTCAGAAA TTATTTTACT ATTTTTTATA AAATAAAAGG |
| 9001 | GAGAAAATGG CTATTTAAAT ACTAGCCTAT TTTATTTCAA TTTTAGCTTA AAATCAGCCC |
| 9061 | CAATTAGCCC CAATTTCAAA TTCAAATGGT CCAGCCCAAT TCCTAAATAA CCCACCCCTA |
| 9121 | ACCCGCCCGG TTTCCCCTTT TGATCCATGC AGTCAACGCC CAGAATTTCC CTATATAATT |
| 9181 | TTTTAATTCC CAAACACCCC TAACTCTATC CCATTTCTCA CCAACCGCCA CATAGATCTA |
| 9241 | TCCTCTTATC TCTCAAACTC TCTCGAACCT TCCCCTAACC CTAGCAGCCT CTCATCATCC |
| 9301 | TCACCTCAAA ACCCACCGGG GCCGGCCATG ATTGAACAAG ATGGATTGCA CGCAGGTTCT |
| 9361 | CCGGCCGCTT GGGTGGAGAG GCTATTCGGC TATGACTGGG CACAACAGAC AATCGGCTGC |
| 9421 | TCTGATGCCG CCGTGTTCCG GCTGTCAGCG CAGGGGAGGC CGGTTCTTTT TGTCAAGACC |
| 9481 | GACCTGTCCG GTGCCCTGAA TGAACTTCAA GACGAGGCAG CGCGGCTATC GTGGCTGGCC |
| 9541 | ACGACGGGCG TTCCTTGCGC AGCTGTGCTC GACGTTGTCA CTGAAGCGGG AAGGGACTGG |
| 9601 | CTGCTATTGG GCGAAGTGCC GGGGCAGGAT CTCCTGTCAT CTCACCTTGC TCCTGCCGAG |
| 9661 | AAAGTATCCA TCATGGCTGA TGCAATGCGG CGGCTGCATA CGCTTGATCC GGCTACCTGC |
| 9721 | CCATTCGACC ACCAAGCGAA ACATCGCATC GAGCGAGCAC GTACTCGGAT GGAAGCCGGT |
| 9781 | CTTGTCGATC AGGATGATCT GGACGAAGAG CATCAGGGGC TCGCGCCAGC CGAACTGTTC |
| 9841 | GCCAGGCTCA AGGCGCGCAT GCCCGACGGC GAGGATCTCG TCGTGACTCA TGGCGATGCC |
| 9901 | TGCTTGCCGA ATATCATGGT GGAAAATGGC CGCTTTTCTG GATTCATCGA CTGTGGCCGG |
| 9961 | CTGGGTGTGG CGGACCGCTA TCAGGACATA GCGTTGGCTA CCCGTGATAT TGCTGAAGAG |
| 10021 | CTTGGCGGCG AATGGGCTGA CCGCTTCCTC GTGCTTTACG GTATCGCCGC TCCCGATTCG |
| 10081 | CAGCGCATCG CCTTCTATCG CCTTCTTGAC GAGTTCTTCT GAGGCGCGCC |
| pORE_R2 pSAG12: GUS 10524 bases | |
| SEQ ID No. 18 | |
| 1 | GATCGTTCAA ACATTTGGCA ATAAAGTTTC TTAAGATTGA ATCCTGTTGC CGGTCTTGCG |
| 61 | ATGATTATCA TATAATTTCT GTTGAATTAC GTTAAGCATG TAATAATTAA CATGTAATGC |
| 121 | ATGACGTTAT TTATGAGATG GGTTTTTATG ATTAGAGTCC CGCAATTATA CATTTAATAC |
| 181 | GCGATAGAAA ACAAAATATA GCGCGCAAAC TAGGATAAAT TATCGCGCGC GGTGTCATCT |
| 241 | ATGTTACTAG ATCCCTAGGG AAGTTCCTAT TCCGAAGTTC CTATTCTCTG AAAAGTATAG |
| 301 | GAACTTCTTT GCGTATTGGG CGCTCTTGGC CTTTTTGGCC ACCGGTCGTA CGGTTAAAAC |
| 361 | CACCCCAGTA CATTAAAAAC GTCCGCAATG TGTTATTAAG TTGTCTAAGC GTCAATTTGT |
| 421 | TTACACCACA ATATATCCTG CCACCAGCCA GCCAACAGCT CCCCGACCGG CAGCTCGGCA |
| 481 | CAAAATCACC ACTCGATACA GGCAGCCCAT CAGTCCACTA GACGCTCACC GGGCTGGTTG |
| 541 | CCCTCGCCGC TGGGCTGGCG GCCGTCTATG GCCCTGCAAA CGCGCCAGAA ACGCCGTCGA |
| 601 | AGCCGTGTGC GAGACACCGC AGCCGCCGGC GTTGTGGATA CCTCGCGGAA AACTTGGCCC |
| 661 | TCACTGACAG ATGAGGGGCG GACGTTGACA CTTGAGGGGC CGACTCACCC GGCGCGGCGT |
| 721 | TGACAGATGA GGGGCAGGCT CGATTTCGGC CGGCGACGTG GAGCTGGCCA GCCTCGCAAA |
| 781 | TCGGCGAAAA CGCCTGATTT TACGCGAGTT TCCCACAGAT GATGTGGACA AGCCTGGGGA |
| 841 | TAAGTGCCCT GCGGTATTGA CACTTGAGGG GCGCGACTAC TGACAGATGA GGGGCGCGAT |
| 901 | CCTTGACACT TGAGGGGCAG AGTGCTGACA GATGAGGGGC GCACCTATTG ACATTTGAGG |
| 961 | GGCTGTCCAC AGGCAGAAAA TCCAGCATTT GCAAGGGTTT CCGCCCGTTT TTCGGCCACC |
| 1021 | GCTAACCTGT CTTTTAACCT GCTTTTAAAC CAATATTTAT AAACCTTGTT TTTAACCAGG |
| 1081 | GCTGCGCCCT GTGCGCGTGA CCGCGCACGC CGAAGGGGGG TGCCCCCCCT TCTCGAACCC |
| 1141 | TCCCGGCCCG CTCTCGCGTT GGCAGCATCA CCCATAATTG TGGTTTCAAA ATCGGCTCCG |
| 1201 | TCGATACTAT GTTATACGCC AACTTTGAAA ACAACTTTGA AAAAGCTGTT TTCTGGTATT |
| 1261 | TAAGGTTTTA GAATGCAAGG AACAGTGAAT TGGAGTTCGT CTTGTTATAA TTAGCTTCTT |
| 1321 | GGGGTATCTT TAAATACTGT AGAAAAGAGG AAGGAAATAA TAAATGGCTA AAATGAGAAT |
| 1381 | ATCACCGGAA TTGAAAAAAC TGATCGAAAA ATACCGCTGC GTAAAAGATA CGGAAGGAAT |
| 1441 | GTCTCCTGCT AAGGTATATA AGCTGGTGGG AGAAAATGAA AACCTATATT TAAAAATGAC |
| 1501 | GGACAGCCGG TATAAAGGGA CCACCTATGA TGTGGAACGG GAAAAGGACA TGATGCTATG |
| 1561 | GCTGGAAGGA AAGCTGCCTG TTCCAAAGGT CCTGCACTTT GAACGGCATG ATGGCTGGAG |
| 1621 | CAATCTGCTC ATGAGTGAGG CCGATGGCGT CCTTTGCTCG GAAGAGTATG AAGATGAACA |
| 1681 | AAGCCCTGAA AAGATTATCG AGCTGTATGC GGAGTGCATC AGGCTCTTTC ACTCCATCGA |
| 1741 | CATATCGGAT TGTCCCTATA CGAATAGCTT AGACAGCCGC TTAGCCGAAT TGGATTACTT |
| 1801 | ACTGAATAAC GATCTGGCCG ATGTGGATTG CGAAAACTGG GAAGAAGACA CTCCATTTAA |
| 1861 | AGATCCGCGC GAGCTGTATG ATTTTTTAAA GACGGAAAAG CCCGAAGAGG AACTTGTCTT |
| 1921 | TTCCCACGGC GACCTGGGAG ACAGCAACAT CTTTGTGAAA GATGGCAAAG TAAGTGGCTT |
| 1981 | TATTGATCTT GGGAGAAGCG GCAGGGCGGA CAAGTGGTAT GACATTGCCT TCTGCGTCCG |
| 2041 | GTCGATCAGG GAGGATATTG GGGAAGAACA GTATGTCGAG CTATTTTTTG ACTTACTGGG |
| 2101 | GATCAAGCCT GATTGGGAGA AAATAAAATA TTATATTTTA CTGGATGAAT TGTTTTAGTA |
| 2161 | CCTAGATGTG GCGCAACGAT GCCGGCGACA AGCAGGAGCG CACCGACTTC TTCCGCATCA |
| 2221 | AGTGTTTTGG CTCTCAGGCC GAGGCCCACG GCAAGTATTT GGGCAAGGGG TCGCTGGTAT |
| 2281 | TCGTGCAGGG CAAGATTCGG AATACCAAGT ACGAGAAGGA CGGCCAGACG GTCTACGGGA |
| 2341 | CCGACTTCAT TGCCGATAAG GTGGATTATC TGGACACCAA GGCACCAGGC GGGTCAAATC |
| 2401 | AGGAATAAGG GCACATTGCC CCGGCGTGAG TCGGGGCAAT CCCGCAAGGA GGGTGAATGA |
| 2461 | ATCGGACGTT TGACCGGAAG GCATACAGGC AAGAACTGAT CGACGCGGGG TTTTCCGCCG |
| 2521 | AGGATGCCGA AACCATCGCA AGCCGCACCG TCATGCGTGC GCCCCGCGAA ACCTTCCAGT |
| 2581 | CCGTCGGCTC GATGGTCCAG CAAGCTACGG CCAAGATCGA GCGCGACAGC GTGCAACTGG |
| 2641 | CTCCCCCTGC CCTGCCCGCG CCATCGGCCG CCGTGGAGCG TTCGCGTCGT CTCGAACAGG |
| 2701 | AGGCGGCAGG TTTGGCGAAG TCGATGACCA TCGACACGCG AGGAACTATG ACGACCAAGA |
| 2761 | AGCGAAAAAC CGCCGGCGAG GACCTGGCAA AACAGGTCAG CGAGGCCAAG CAAGCCGCGT |
| 2821 | TGCTGAAACA CACGAAGCAG CAGATCAAGG AAATGCAGCT TTCCTTGTTC GATATTGCGC |
| 2881 | CGTGGCCGGA CACGATGCGA GCGATGCCAA ACGACACGGC CCGCTCTGCC CTGTTCACCA |
| 2941 | CGCGCAACAA GAAAATCCCG CGCGAGGCGC TGCAAAACAA GGTCATTTTC CACGTCAACA |
| 3001 | AGGACGTGAA GATCACCTAC ACCGGCGTCG AGCTGCGGGC CGACGATGAC GAACTGGTGT |
| 3061 | GGCAGCAGGT GTTGGAGTAC GCGAAGCGCA CCCCTATCGG CGAGCCGATC ACCTTCACGT |
| 3121 | TCTACGAGCT TTGCCAGGAC CTGGGCTGGT CGATCAATGG CCGGTATTAC ACGAAGGCCG |
| 3181 | AGGAATGCCT GTCGCGCCTA CAGGCGACGG CGATGGGCTT CACGTCCGAC CGCGTTGGGC |
| 3241 | ACCTGGAATC GGTGTCGCTG CTGCACCGCT TCCGCGTCCT GGACCGTGGC AAGAAAACGT |
| 3301 | CCCGTTGCCA GGTCCTGATC GACGAGGAAA TCGTCGTGCT GTTTGCTGGC GACCACTACA |
| 3361 | CGAAATTCAT ATGGGAGAAG TACCGCAAGC TGTCGCCGAC GGCCCGACGG ATGTTCGACT |
| 3421 | ATTTCAGCTC GCACCGGGAG CCGTACCCGC TCAAGCTGGA AACCTTCCGC CTCATGTGCG |
| 3481 | GATCGGATTC CACCCGCGTG AAGAAGTGGC GCGAGCAGGT CGGCGAAGCC TGCGAAGAGT |
| 3541 | TGCGAGGCAG CGGCCTGGTG GAACACGCCT GGGTCAATGA TGACCTGGTG CATTGCAAAC |
| 3601 | GCTAGGGCCT TGTGGGGTCA GTTCCGGCTG GGGGTTCAGC AGCCAGCGCT TTACTGAGAT |
| 3661 | CCTCTTCCGC TTCCTCGCTC ACTGACTCGC TGCGCTCGGT CGTTCGGCTG CGGCGAGCGG |
| 3721 | TATCAGCTCA CTCAAAGGCG GTAATACGGT TATCCACAGA ATCAGGGGAT AACGCAGGAA |
| 3781 | AGAACATGTG AGCAAAAGGC CAGCAAAAGG CCAGGAACCG TAAAAAGGCC GCGTTGCTGG |
| 3841 | CGTTTTTCCA TAGGCTCCGC CCCCCTGACG AGCATCACAA AAATCGACGC TCAAGTCAGA |
| 3901 | GGTGGCGAAA CCCGACAGGA CTATAAAGAT ACCAGGCGTT TCCCCCTGGA AGCTCCCTCG |
| 3961 | TGCGCTCTCC TGTTCCGACC CTGCCGCTTA CCGGATACCT GTCCGCCTTT CTCCCTTCGG |
| 4021 | GAAGCGTGGC GCTTTCTCAT AGCTCACGCT GTAGGTATCT CAGTTCGGTG TAGGTCGTTC |
| 4081 | GCTCCAAGCT GGGCTGTGTG CACGAACCCC CCGTTCAGCC CGACCGCTGC GCCTTATCCG |
| 4141 | GTAACTATCG TCTTGAGTCC AACCCGGTAA GACACGACTT ATCGCCACTG GCAGCAGCCA |
| 4201 | CTGGTAACAG GATTAGCAGA GCGAGGTATG TAGGCGGTGC TACAGAGTTC TTGAAGTGGT |
| 4261 | GGCCTAACTA CGGCTACACT AGAAGAACAG TATTTGGTAT CTGCGCTCTG CTGAAGCCAG |
| 4321 | TTACCTTCGG AAAAAGAGTT GGTAGCTCTT GATCCGGCAA ACAAACCACC GCTGGTAGCG |
| 4381 | GTGGTTTTTT TGTTTGCAAG CAGCAGATTA CGCGCAGAAA AAAAGGATCT CAAGAAGATC |
| 4441 | CTTTGATCTT TTCTACGGGG TCTGACGCTC AGTGGAACGA AAACTCACGT TAAGGGATTT |
| 4501 | TGGTCATGAG ATTATCAAAA AGGATCTTCA CCTAGATCCT TTTGGATCTC CTGTGGTTGG |
| 4561 | CATGCACATA CAAATGGACG AACGGATAAA CCTTTTCACG CCCTTTTAAA TATCCGATTA |
| 4621 | TTCTAATAAA CGCTCTTTTC TCTTAGGTTT ACCCGCCAAT ATATCCTGTC AAACACTGAT |
| 4681 | AGTTTAAACT GAAGGCGGGA AACGACAATC TGCTAGTGGA TCTCCCAGTC ACGACGTTGT |
| 4741 | AAAACGGGCG CCCCGCGGga tatctctttt tatattcaaa caataagttg agatatgttt |
| 4801 | gagaagagga caactattct cgtggagcac cgagtttgtt ttatattaga aacccgattg |
| 4861 | ttatttttag actgagacaa aaaagtaaaa tcgttgattg ttaaaattta aaattagttt |
| 4921 | cattacgttt cgataaaaaa atgattagtt tatcatagct taattatagc attgatttct |
| 4981 | aaattagttt tttgaccacc cttttttctc tctttggtgt tttcttaaca ttagaagaac |
| 5041 | ccataacaat gtacgttcaa attaattaaa aacaatattt ccaagtttta tatacgaaac |
| 5101 | ttgttttttt taatgaaaac agttgaatag ttgattatga attagttaga tcaatactca |
| 5161 | atatatgatc aatgatgtat atatatgaac tcagttgtta tacaagaaat gaaaatgcta |
| 5221 | tttaaatacc gatcatgaag tgttaaaaag tgtcagaata tgacatgaag cgttttgtcc |
| 5281 | taccgggtat tcgagttata ggtttggatc tctcaagaat attttgggcc atattagtta |
| 5341 | tatttgggct taagcgtttt gcaaagagac gaggaagaaa gattgggtca agttaacaaa |
| 5401 | acagagacac tcgtattagt tggtactttg gtagcaagtc gatttatttg ccagtaaaaa |
| 5461 | cttggtacac aactgacaac tcgtatcgtt attagtttgt acttggtacc tttggttcaa |
| 5521 | gaaaaagttg atatagttaa atcagttgtg ttcatgaggt gattgtgatt taatttgttg |
| 5581 | actagggcga ttccttcaca tcacaataac aaagttttat agattttttt tttataacat |
| 5641 | ttttgccacg cttcgtaaag tttggtattt acaccgcatt tttccctgta caagaattca |
| 5701 | tatattattt atttatatac tccagttgac aattataagt ttataacgtt tttacaatta |
| 5761 | tttaaatacc atgtgaagat ccaagaatat gtcttacttc ttctttgtgt aagaaaacta |
| 5821 | actatatcac tataataaaa taattctaat cattatattt gtaaatatgc attagtttgt |
| 5881 | caattttgaa tttagtattt tagacgttat cacttcagcc aaatatgatt tggatttaag |
| 5941 | tccaaaatgc aatttcgtac gtatccctct tgtcgtctaa tgattatttc aatatttctt |
| 6001 | atattatccc taactacaga gctacattta tattgtattc taatgacagg gaaactttca |
| 6061 | tagagattca gatagatgaa attggtggga aacatcattg aacaggaaac ttttagcaaa |
| 6121 | tcatatcgat ttatctacaa aagaatactt agcgtaatga agttcacttg ttgtgaatga |
| 6181 | ctatgatttg atcaaattag ttaattttgt cgaatcattt ttctttttga tttgattaag |
| 6241 | cttttaactt gcacgaatgg ttctcttgtg aataaacaga atctttgaat tcaaactatt |
| 6301 | tgattagtga aaagacaaaa gaagattcct tgtttttatg tgattagtga ttttgatgca |
| 6361 | tgaaaggtac ctacgtacta caagaaaaat aaacatgtac gtaactacgt atcagcatgt |
| 6421 | aaaagtattt ttttccaaat aatttatact catgatagat tttttttttt tgaaatgtca |
| 6481 | attaaaaatg ctttcttaaa tattaatttt aattaattaa ataaggaaat atatttatgc |
| 6541 | aaaacatcat caacacatat ccaacttcga aaatctctat agtacacaag tagagaaatt |
| 6601 | aaattttact agatacaaac ttcctaatca tcaaatataa atgtttacaa aactaattaa |
| 6661 | acccaccact aaaattaact aaaaatccga gcaaagtgag tgaacaagac ttgatttcag |
| 6721 | gttgatgtag gactaaaatg gctacgtatc aaacatcaac gatcatttag ttatgtatga |
| 6781 | atgaatgtag tcattacttg taaaacaaaa atgctttgat ttggatcaat cacttcatgt |
| 6841 | gaacattagc aattacatca accttatttt cactataaaa ccccatctca gtacccttct |
| 6901 | gaagtaatca aattaagagc aaaagtcatt taactttcct aaaacaCTCG AGTTTTTCTA |
| 6961 | GAAGGCCTTG GATCCACCCG GGAGAATTCG TCGACTTTGC GGCCGCATCG ATACTGCAGG |
| 7021 | AGCTCATGTT ACGTCCTGTA GAAACCCCAA CCCGTGAAAT CAAAAAACTC GACGGCCTGT |
| 7081 | GGGCATTCAG TCTGGATCGC GAAAACTGTG GAATTGATCA GCGTTGGTGG GAAAGCGCGT |
| 7141 | TACAAGAAAG CCGGGCTATT GCTGTGCCAG GCAGTTTTAA CGATCAGTTC GCCGATGCAG |
| 7201 | ATATTCGTAA TTATGCGGGC AACGTCTGGT ATCAGCGCGA AGTCTTTATA CCGAAAGGTT |
| 7261 | GGGCAGGCCA GCGTATCGTG CTGCGTTTCG ATGCGGTCAC TCATTACGGC AAAGTGTGGG |
| 7321 | TCAATAATCA GGAAGTGATG GAGCATCAGG GCGGCTATAC GCCATTTGAA GCCGATGTCA |
| 7381 | CGCCGTATGT TATTGCCGGG AAAAGTGTAC GTATCACCGT TTGTGTGAAC AACGAACTGA |
| 7441 | ACTGGCAGAC TATCCCGCCG GGAATGGTGA TTACCGACGA AAACGGCAAG AAAAAGCAGT |
| 7501 | CTTACTTCCA TGATTTCTTT AACTATGCCG GAATCCATCG CAGCGTAATG CTCTACACCA |
| 7561 | CGCCGAACAC CTGGGTGGAC GATATTACCG TGGTGACGCA TGTCGCGCAA GACTGTAACC |
| 7621 | ACGCTTCTGT TGACTGGCAG GTGGTGGCCA ATGGTGATGT CAGCGTTGAA CTGCGTGATG |
| 7681 | CGGATCAACA GGTGGTTGCA ACTGGACAAG GCACTAGCGG GACTTTGCAA GTGGTGAATC |
| 7741 | CGCACCTCTG GCAACCGGGT GAAGGTTATC TCTATGAACT GTGCGTCACA GCCAAAAGCC |
| 7801 | AGACAGAGTG TGATATTTAC CCGCTTCGCG TCGGCATCCG GTCAGTGGCA GTGAAGGGCG |
| 7861 | AACAGTTCCT GATTAACCAC AAACCGTTCT ACTTTACTGG CTTTGGTCGT CATGAAGATG |
| 7921 | CGGACTTGCG TGGCAAAGGA TTCGATAACG TGCTGATGGT GCACGACCAC GCATTAATGG |
| 7981 | ACTGGATTGG GGCCAACTCC TACCGTACCT CGCATTACCC TTACGCTGAA GAGATGCTCG |
| 8041 | ACTGGGCAGA TGAACATGGC ATCGTGGTGA TTGATGAAAC TGCTGCTGTC GGCTTTAACC |
| 8101 | TCTCTTTAGG CATTGGTTTC GAAGCGGGCA ACAAGCCGAA AGAACTGTAC AGCGAAGAGG |
| 8161 | CAGTCAACGG GGAAACTCAG CAAGCGCACT TACAGGCGAT TAAAGAGCTG ATAGCGCGTG |
| 8221 | ACAAAAACCA CCCAAGCGTG GTGATGTGGA GTATTGCCAA CGAACCGGAT ACCCGTCCGC |
| 8281 | AAGGTGCACG GGAATATTTC GCGCCACTGG CGGAAGCAAC CCGTAAACTC GACCCGACCC |
| 8341 | GTCCGATCAC CTGCGTCAAT GTAATGTTCT GCGACGCTCA CACCGATACC ATCAGCGATC |
| 8401 | TCTTTGATGT GCTGTGCCTG AACCGTTATT ACGGATGGTA TGTCCAAAGC GGCGATTTGG |
| 8461 | AAACGGCAGA GAAGGTACTG GAAAAAGAAC TTCTGGCCTG GCAGGAGAAA CTGCATCAGC |
| 8521 | CGATTATCAT CACCGAATAC GGCGTGGATA CGTTAGCCGG GCTGCACTCA ATGTACACCG |
| 8581 | ACATGTGGAG TGAAGAGTAT CAGTGTGCAT GGCTGGATAT GTATCACCGC GTCTTTGATC |
| 8641 | GCGTCAGCGC CGTCGTCGGT GAACAGGTAT GGAATTTCGC CGATTTTGCG ACCTCGCAAG |
| 8701 | GCATATTGCG CGTTGGCGGT AACAAGAAAG GGATCTTCAC TCGCGACCGC AAACCGAAGT |
| 8761 | CGGCGGCTTT TCTGCTGCAA AAACGCTGGA CTGGCATGAA CTTCGGTGAA AAACCGCAGC |
| 8821 | AGGGAGGCAA ACAATGAGGT ACCTTTTACT AGTGATATCC CTGTGTGAAA TTGTTATCCG |
| 8881 | CTACGCGTGA TCGTTCAAAC ATTTGGCAAT AAAGTTTCTT AAGATTGAAT CCTGTTGCCG |
| 8941 | GTCTTGCGAT GATTATCATA TAATTTCTGT TGAATTACGT TAAGCATGTA ATAATTAACA |
| 9001 | TGTAATGCAT GACGTTATTT ATGAGATGGG TTTTTATGAT TAGAGTCCCG CAATTATACA |
| 9061 | TTTAATACGC GATAGAAAAC AAAATATAGC GCGCAAACTA GGATAAATTA TCGCGCGCGG |
| 9121 | TGTCATCTAT GTTACTAGAT CCCATGGGAA GTTCCTATTC CGAAGTTCCT ATTCTCTGAA |
| 9181 | AAGTATAGGA ACTTCAGCGA TCGCAGACGT CGGGATCTTC TGCAAGCATC TCTATTTCCT |
| 9241 | GAAGGTCTAA CCTCGAAGAT TTAAGATTTA ATTACGTTTA TAATTACAAA ATTGATTCTA |
| 9301 | GTATCTTTAA TTTAATGCTT ATACATTATT AATTAATTTA GTACTTTCAA TTTGTTTTCA |
| 9361 | GAAATTATTT TACTATTTTT TATAAAATAA AAGGGAGAAA ATGGCTATTT AAATACTAGC |
| 9421 | CTATTTTATT TCAATTTTAG CTTAAAATCA GCCCCAATTA GCCCCAATTT CAAATTCAAA |
| 9481 | TGGTCCAGCC CAATTCCTAA ATAACCCACC CCTAACCCGC CCGGTTTCCC CTTTTGATCC |
| 9541 | ATGCAGTCAA CGCCCAGAAT TTCCCTATAT AATTTTTTAA TTCCCAAACA CCCCTAACTC |
| 9601 | TATCCCATTT CTCACCAACC GCCACATAGA TCTATCCTCT TATCTCTCAA ACTCTCTCGA |
| 9661 | ACCTTCCCCT AACCCTAGCA GCCTCTCATC ATCCTCACCT CAAAACCCAC CGGGGCCGGC |
| 9721 | CATGATTGAA CAAGATGGAT TGCACGCAGG TTCTCCGGCC GCTTGGGTGG AGAGGCTATT |
| 9781 | CGGCTATGAC TGGGCACAAC AGACAATCGG CTGCTCTGAT GCCGCCGTGT TCCGGCTGTC |
| 9841 | AGCGCAGGGG AGGCCGGTTC TTTTTGTCAA GACCGACCTG TCCGGTGCCC TGAATGAACT |
| 9901 | TCAAGACGAG GCAGCGCGGC TATCGTGGCT GGCCACGACG GGCGTTCCTT GCGCAGCTGT |
| 9961 | GCTCGACGTT GTCACTGAAG CGGGAAGGGA CTGGCTGCTA TTGGGCGAAG TGCCGGGGCA |
| 10021 | GGATCTCCTG TCATCTCACC TTGCTCCTGC CGAGAAAGTA TCCATCATGG CTGATGCAAT |
| 10081 | GCGGCGGCTG CATACGCTTG ATCCGGCTAC CTGCCCATTC GACCACCAAG CGAAACATCG |
| 10141 | CATCGAGCGA GCACGTACTC GGATGGAAGC CGGTCTTGTC GATCAGGATG ATCTGGACGA |
| 10201 | AGAGCATCAG GGGCTCGCGC CAGCCGAACT GTTCGCCAGG CTCAAGGCGC GCATGCCCGA |
| 10261 | CGGCGAGGAT CTCGTCGTGA CTCATGGCGA TGCCTGCTTG CCGAATATCA TGGTGGAAAA |
| 10321 | TGGCCGCTTT TCTGGATTCA TCGACTGTGG CCGGCTGGGT GTGGCGGACC GCTATCAGGA |
| 10381 | CATAGCGTTG GCTACCCGTG ATATTGCTGA AGAGCTTGGC GGCGAATGGG CTGACCGCTT |
| 10441 | CCTCGTGCTT TACGGTATCG CCGCTCCCGA TTCGCAGCGC ATCGCCTTCT ATCGCCTTCT |
| 10501 | TGACGAGTTC TTCTGAGGCG CGCC |
| pSAG2 promoter, 1066 bp (AT5G60360) | |
| SEQ ID No. 19 | |
| 1 | tcctaccatt ctcccatctt tttaatttgt atgccttata cttaatttta attggttcaa |
| 61 | tcgtcatact tcatgaataa gatgtttcct tcaaatatat aaacgatgca gtgtgtaatt |
| 121 | tgcagtcggt gtcggatttc gcacacattc gactcaacga aattaatgtt ttgaaaattt |
| 181 | aaattggaca tcgaaaattt atattcattg gatatatgtg tgtcccaaat aaaagtaaac |
| 241 | aagaatgtag aacacatcaa aataattaat atcacttatc ctgcaacaaa atgagaaaac |
| 301 | aacaaaatta gatcataaaa tatattattc aaaattatac ttagtaaatt taatggtaaa |
| 361 | agatcaaata tgacttgctt ttcaaaatgt taaaacagtc tcgaatacgt aaaataattt |
| 421 | gatcacaaaa taaagtatat agataacaag attttctaat ctatatcaaa ttggaaaata |
| 481 | ttttttcata acagtgaaaa gatttttttt taaaagtttg tacattgatt tttgattcaa |
| 541 | aaaccttagt taattaaaag atctcttacc aataaaagtt tctgaatggg atttggtatt |
| 601 | gaactgtgtc cttaaatccg ggttttagtt caaccacgag tttaactaca taaacgattt |
| 661 | ccggtcagcc tggtcctgac gggccaccaa ttttcaaaac attgacgaaa gcaagcccaa |
| 721 | taacatcaaa tataattaac aaattttagt ttgaatacgg ttactgtttt ggaaattgat |
| 781 | acattccgat cttggactaa tctataatcc atgggctggc tattttgcag tttcccgtaa |
| 841 | ataaaattat gaaaacatga ttgatttctc gttggtagac catgacatca caggacacgt |
| 901 | caacattcca agtataaagg acaaaactgt ccatttagat tccaccacgt gtcccgataa |
| 961 | cgtttccgtc tgtatctctc tcttccgtct tctactcttc ttcttcttct tcttcttcca |
| 1021 | ctcacaatct atcgagctaa aacactgaag cagacattag cgttga |
| Sequence amplified by PCR from screening transgenic potato plants | |
| using primer sequences Forward: 5′-AATCAGTTGTGTTCATGAGGTG-3′ | |
| SEQ ID No. 91; and Reverse: 5′-AGCGGATAACAATTTCACACAGG-3′ | |
| SEQ ID No. 92, 2949 bp. | |
| SEQ ID No. 20 | |
| 1 | aatcagttgt gttcatgagg tgattgtgat ttaatttgtt gactagggcg attccttcac |
| 61 | atcacaataa caaagtttta tagatttttt ttttataaca tttttgccac gcttcgtaaa |
| 121 | gtttggtatt tacaccgcat ttttccctgt acaagaattc atatattatt tatttatata |
| 181 | ctccagttga caattataag tttataacgt ttttacaatt atttaaatac catgtgaaga |
| 241 | tccaagaata tgtcttactt cttctttgtg taagaaaact aactatatca ctataataaa |
| 301 | ataattctaa tcattatatt tgtaaatatg cagttatttg tcaattttga atttagtatt |
| 361 | ttagacgtta tcacttcagc caaatatgat ttggatttaa gtccaaaatg caatttcgta |
| 421 | cgtatccctc ttgtcgtcta atgattattt caatatttct tatattatcc ctaactacag |
| 481 | agctacattt atattgtatt ctaatgacag ggaaactttc atagagattc agatagatga |
| 541 | aattggtggg aaacatcatt gaacaggaaa cttttagcaa atcatatcga tttatctaca |
| 601 | aaagaatact tagcgtaatg aagttcactt gttgtgaatg actatgattt gatcaaatta |
| 661 | gttaattttg tcgaatcatt tttctttttg atttgattaa gcttttaact tgcacgaatg |
| 721 | gttctcttgt gaataaacag aatctttgaa ttcaaactat ttgattagtg aaaagacaaa |
| 781 | agaagattcc ttgtttttat gtgattagtg attttgatgc atgaaaggta cctacgtact |
| 841 | acaagaaaaa taaacatgta cgtaactacg tatcagcatg taaaagtatt tttttccaaa |
| 901 | taatttatac tcatgataga tttttttttt ttgaaatgtc aattaaaaat gctttcttaa |
| 961 | atattaattt taattaatta aataaggaaa tatatttatg caaaacatca tcaacacata |
| 1021 | tccaacttcg aaaatctcta tagtacacaa gtagagaaat taaattttac tagatacaaa |
| 1081 | cttcctaatc atcaaatata aatgtttaca aaactaatta aacccaccac taaaattaac |
| 1141 | taaaaatccg agcaaagtga gtgaacaaga cttgatttca ggttgatgta ggactaaaat |
| 1201 | ggctacgtat caaacatcaa cgatcattta gttatgtatg aatgaatgta gtcattactt |
| 1261 | gtaaaacaaa aatgctttga tttggatcaa tcacttcatg tgaacattag caattacatc |
| 1321 | aaccttattt tcactataaa accccatctc agtacccttc tgaagtaatc aaattaagag |
| 1381 | caaaagtcat ttaactttcc taaaacaCTC GAGGTATTTT TACAACAATT ACCAACAACA |
| 1441 | ACCAACAACA AACAACATTA CAATTACTAT TTACAATTAC AATTACCATG GACTACAAGG |
| 1501 | ACGACGATGA CAAGCATATG ACAAAGAAGA GAAAGCTCGA ATCTGAATCC AACGAAACGT |
| 1561 | CAGAGCCGAC GGAGAAGCAG CAGCAGCAAT GTGAAAAAGA GGATCCGGAA ATCAGAAATG |
| 1621 | TTGATAATCA AAGAGACGAC GACGAACAAG TAGTAGAGCA AGACACACTA AAGGAGATGC |
| 1681 | ACGAAGAGGA AGCTAAAGGT GAAGATAACA TAGAAGCGGA GACGTCGTCC GGATCCGGAA |
| 1741 | ATCAAGGAAA TGAGGATGAT GACGAAGAAG AACCTATTGA GGATCTATTG GAACCGTTTT |
| 1801 | CAAAGGATCA ACTTTTGATT CTTCTCAAGG AAGCTGCAGA GAGACATCGC GATGTAGCTA |
| 1861 | ATCGAATCCG GATTGTGGCG GATGAAGATC TTGTTCATCG TAAGATCTTC GTTCACGGGC |
| 1921 | TTGGATGGGA TACTAAAGCT GATTCACTTA TCGACGCTTT TAAACAGTAC GGAGAGATTG |
| 1981 | AAGATTGCAA ATGTGTGGTT GATAAGGTAT CTGGGCAATC TAAAGGTTAT GGCTTTATCC |
| 2041 | TCTTTAAGTC AAGGTCTGGT GCTCGTAACG CTCTTAAGCA GCCTCAGAAG AAGATTGGAA |
| 2101 | CTCGTATGAC TGCGTGCCAG CTGGCGTCTA TAGGACCTGT TCAGGGGAAC CCTGTTGTGG |
| 2161 | CTCCTGCTCA GCATTTCAAT CCTGAGAATG TTCAGAGGAA GATTTATGTC AGTAACGTTA |
| 2221 | GTGCAGACAT TGATCCGCAG AAGTTGCTGG AGTTTTTCTC AAGGTTTGGG GAGATAGAAG |
| 2281 | AAGGTCCTTT GGGGCTTGAT AAAGCTACTG GGAGACCTAA AGGTTTCGCT TTGTTTGTCT |
| 2341 | ATAGATCCCT AGAAAGTGCC AAGAAGGCAT TGGAGGAGCC ACACAAGACT TTTGAAGGCC |
| 2401 | ATGTCTTGCA TTGCCACAAA GCAAATGATG GGCCAAAACA GGTTAAGCAA CATCAACATA |
| 2461 | ACCATAACTC TCACAATCAA AATTCCCGTT ACCAAAGGAA CGACAACAAT GGTTATGGTG |
| 2521 | CCCCTGGAGG CCATGGACAT TTCATAGCTG GTAATAACCA AGCTGTGCAG GCGTTTAATC |
| 2581 | CGGCCATTGG CCAGGCCCTC ACAGCTTTGC TGGCATCTCA GGGTGCTGGG TTGGGTTTAA |
| 2641 | ACCAAGCATT TGGGCAGGCT TTGTTGGGGA CATTAGGGAC AGCTAGCCCA GGAGCTGTAG |
| 2701 | GTGGAATGCC AAGTGGCTAT GGTACTCAAG CAAATATCTC ACCTGGGGTC TATCCTGGGT |
| 2761 | ACGGTGCTCA AGCCGGGTAC CAGGGCGGTT ATCAGACTCA GCAACCTGGT CAGGGCGGTG |
| 2821 | CGGGAAGAGG GCAGCATGGT GCTGGGTATG GTGGTCCTTA CATGGGTCGT TAGATTAGCC |
| 2881 | ACTCAGGAAG GGCGAATTCG TTTAAACCTG CAGGACTAGT GATATCCCTG TGTGAAATTG |
| 2941 | TTATCCGCT |
| RNA-recognition motif 1 (RRM1) of Arabidopsis AKIP1 | |
| SEQ ID No. 21 | |
| 1 | KIFVHGLGWDTKTETLIEAFKQYGEIEDCKAVFDKISGKSKGYGFILYKSRSGARNALKQ |
| RNA-recognition motif 1 (RRM1) of Arabidopsis AKIP2 | |
| SEQ ID No. 22 | |
| 1 | KIFVHGLGWDTKADSLIDAFKQYGEIEDCKCVVDKVSGQSKGYGFILFKSRSGARNALKQ |
| RNA-recognition motif 1 (RRM1) of Arabidopsis AKIP3 | |
| SEQ ID No. 23 | |
| 1 | KLFIRGLAADTTTEGLRSLFSSYGDLEEAIVILDKVTGKSKGYGFVTFMHVDGALLALKE |
| RNA-recognition motif 1 (RRM1) of Potato AKIP1/2 (StAKIP1/2) | |
| SEQ ID No. 24 | |
| 1 | KIFVHGLGWDTTAETLTSVFATYGEIEDCKAVTDKVSGKSKGYGFILFKHRSGARKALKE |
| RNA-recognition motif 1 (RRM1) of Potato AKIP3 (StAKIP3) | |
| SEQ ID No. 25 | |
| 1 | KLFVRGLGWETTTDKLRQVFSEFGELDEAVVITDKASSRSKGYGFVTFKHVDAAILSLKV |
| RNA-recognition motif 2 (RRM2) of Arabidopsis AKIP1 | |
| SEQ ID No. 26 | |
| 1 | KIYVSNVGAELDPQKLLMFFSKFGEIEEGPLGLDKYTGRPKGFCLFVYKSSESAKRALEE |
| 61 | PHKTFEGHILHCQK |
| RNA-recognition motif 2 (RRM2) of Arabidopsis AKIP2 | |
| SEQ ID No. 27 | |
| 1 | KIYVSNVSADIDPQKLLEFFSRFGEIEEGPLGLDKATGRPKGFALFVYRSLESAKKALEE |
| 61 | PHKTFEGHVLHCHK |
| RNA-recognition motif 2 (RRM2) of Arabidopsis AKIP3 | |
| SEQ ID No. 28 | |
| 1 | KIYVANVPFDMPADRLLNHFMAYGDVEEGPLGFDKVTGKSRGFALFVYKTAEGAQAALAD |
| 61 | PVKVIDGKHLNCKL |
| RNA-recognition motif 2 (RRM2) of Potato AKIP1/2 (StAKIP1/2) | |
| SEQ ID No. 29 | |
| 1 | KIFVSNVAADLEPQKLLEYFSKFGEVEEGPLGLDKQSGKPKGFCLFVYKTVEGARKALEE |
| 61 | PHKTFEGHTLHCQK |
| RNA-recognition motif 2 (RRM2) of Potato AKIP3 (StAKIP3) | |
| SEQ ID No. 30 | |
| 1 | KIYVGNVPFEISSEKLLNHFSMYGEIEEGPLGFDKQTGKAKGFAFFVYKTEDGARASLVD |
| 61 | PVKTVEGHQVLCKL |

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. An expression cassette comprising a nucleic acid encoding an abscisic-acid-activated protein kinase-interacting protein. AKIP, operably linked to a heterologous plant non-constitutive promoter.

2. The expression cassette of claim 1 wherein the heterologous plant non-constitutive promoter is a senescence-activated gene promoter.

3. The expression cassette of claim 1 wherein the heterologous plant non-constitutive promoter is plant cell type-specific promoter.

4. The expression cassette of claim 1 wherein the heterologous plant non-constitutive promoter is guard cell-specific promoter.

5. The expression cassette of claim 1 wherein the abscisic-acid-activated protein kinase-interacting protein is a potato abscisic-acid-activated protein kinase-interacting protein.

6. The expression cassette of claim 1 wherein the abscisic-acid-activated protein kinase-interacting protein is a potato abscisic-acid-activated protein kinase-interacting protein having at least 95% identity to SEQ ID No. 2.

7. The expression cassette of claim 1 wherein the abscisic-acid-activated protein kinase-interacting protein is selected from: a protein comprising the amino acid sequence of SEQ ID No. 2; a protein comprising the amino acid sequence of SEQ ID No. 4; a protein encoded by the complement of a nucleic acid that hybridizes under highly stringent conditions with the nucleotide sequence of SEQ ID No. 1; a protein encoded by the complement of a nucleic acid that hybridizes under highly stringent conditions with the nucleotide sequence of SEQ ID No. 3; wherein the highly stringent conditions are: hybridization in a solution containing 6×SSC, 5× Denhardt's solution, 30% formamide, and 100 micrograms/ml denatured salmon sperm DNA at 37° C. overnight followed by washing in a solution of 0.1×SSC and 0.1% SDS at 60° C. for 15 minutes; a protein comprising an amino acid sequence that is at least 95% identical to SEQ ID No. 2; and a protein comprising an amino acid sequence that is at least 95% identical to SEQ ID No. 4.

8. The expression cassette of claim 1 wherein the abscisic-acid-activated protein kinase-interacting protein is selected from: a protein comprising the amino acid sequence of SEQ ID No. 6; a protein comprising the amino acid sequence of SEQ ID No. 8; a protein comprising the amino acid sequence of SEQ ID No. 10; a protein encoded by the complement of a nucleic acid that hybridizes under highly stringent conditions with the nucleotide sequence of SEQ ID No. 5; a protein encoded by the complement of a nucleic acid that hybridizes under highly stringent conditions with the nucleotide sequence of SEQ ID No. 7; a protein encoded by the complement of a nucleic acid that hybridizes under highly stringent conditions with the nucleotide sequence of SEQ ID No. 9; wherein the highly stringent conditions are: hybridization in a solution containing 6×SSC, 5× Denhardt's solution, 30% formamide, and 100 micrograms/nil denatured salmon sperm DNA at 37° C. overnight followed by washing in a solution of 0.1×SSC and 0.1% SDS at 60° C. for 15 minutes; a protein comprising an amino acid sequence that is at least 95% identical to SEQ ID No. 6; a protein comprising an amino acid sequence that is at least 95% identical to SEQ ID No. 8; and a protein comprising an amino acid sequence that is at least 95% identical to SEQ ID No. 10.

9. The expression cassette of claim 1 wherein the abscisic-acid-activated protein kinase-interacting protein is selected from: a protein comprising the amino acid sequence of SEQ ID No. 24 and the amino acid sequence of SEQ ID No. 29; a protein comprising the amino acid sequence of SEQ ID No. 25 and the amino acid sequence of SEQ ID No. 30; a protein comprising an amino acid sequence that is at least 95% identical to the amino acid sequence of SEQ ID No. 24 and comprising an amino acid sequence that is at least 95% identical to the amino acid sequence of SEQ ID No. 29; and a protein comprising an amino acid sequence that is at least 95% identical to the amino acid sequence of SEQ ID No. 25 and comprising an amino acid sequence that is at least 95% identical to the amino acid sequence of SEQ ID No. 30.

10. The expression cassette of claim 1 wherein the abscisic-acid-activated protein kinase-interacting protein is selected from: a protein comprising the amino acid sequence of SEQ ID No. 21 and the amino acid sequence of SEQ ID No. 26; a protein comprising the amino acid sequence of SEQ ID No. 22 and the amino acid sequence of SEQ ID No. 27; a protein comprising the amino acid sequence of SEQ ID No. 23 and the amino acid sequence of SEQ ID No. 28; a protein comprising an amino acid sequence that is at least 95% identical to the amino acid sequence of SEQ ID No. 21 and comprising an amino acid sequence that is at least 95% identical to the amino acid sequence of SEQ ID No. 26; a protein comprising an amino acid sequence that is at least 95% identical to the amino acid sequence of SEQ ID No. 22 and comprising an amino acid sequence that is at least 95% identical to the amino acid sequence of SEQ ID No. 27; and a protein comprising an amino acid sequence that is at least 95% identical to the amino acid sequence of SEQ ID No. 23 and comprising an amino acid sequence that is at least 95% identical to the amino acid sequence of SEQ ID No. 28.

11. The expression cassette of claim 2 wherein the senescence-activated gene promoter is a 5′ non-coding region of a gene selected from the group consisting of SPG31; SAG2 (At5g60360); SAG12 (At5g45890), SAG13 (At2g29350); SAG14 (At5g20230); SAG15 (At5g51070); SAG101 (At5g14930); SIRK (At2g19190); WRKY6 (At1g62300); WRKY53 (At4g23810); and WRKY70 (At3g56400).

12. The expression cassette of claim 2 wherein the senescence-activated gene promoter comprises a nucleic acid sequence selected from the group consisting of: SEQ ID No. 11, SEQ ID No. 12 and SEQ ID No. 19.

13. The expression cassette of claim 4 wherein the senescence-activated gene promoter comprises the nucleic acid sequence SEQ ID No. 13.

14. A transgenic plant transformed with an expression vector comprising the expression cassette of claim 1.

15. The transgenic plant of claim 14, wherein the plant is a potato plant.

16. The transgenic plant of claim 14, wherein the plant is a cotton plant.

17. The transgenic plant of claim 14, wherein the plant is selected from the group consisting of: alfalfa, apple, apricot, asparagus, avocado, banana, barley, bean, blackberry, broccoli, cabbage, carrot, cauliflower, celery, cherry, chicory, clover, cucumber, eggplant, garlic, grape, hemp, lettuce, maize, mango, melon, miscanthus, nectarine, onion, papaya, pea, peach, pear, pepper, pineapple, plum, pumpkin, quince, radish, rapeseed, rice, raspberry, rye, soybean, sorghum, spinach, squash, strawberry, sugarcane, sugarbeet, sunflower, sweet potato, switchgrass, tobacco, tomato, turnip, wheat, zucchini, aster, basil, bay leaf, begonia, chives, chrysanthemum, cilantro, clover, delphinium, dill, eucalyptus, lavender, lemon grass, mint, oregano, parsley, rosemary, savory, sunflower, tarragon, thyme, and zinnia.

18. A host cell comprising the expression cassette of claim 1.

19. A plant part obtained from the transgenic plant of claim 14.

20. Progeny of the transgenic plant of claim 14.

21. A method of making a transgenic plant having enhanced senescence, comprising introducing the expression cassette of claim 1 into a cell of a plant or a portion of the plant and generating a whole plant from the cell or the portion of the plant.

22. A transgenic plant comprising a plant expression vector comprising a nucleic acid encoding an AKIP, the nucleic acid encoding an AKIP operably linked to a heterologous plant developmental stage-specific and/or cell type-specific promoter, wherein the transgenic plant is characterized by enhanced senescence.

23. A method of harvesting a plant or a useful portion of a plant, comprising increasing expression of an AKIP in the plant, wherein increasing expression of an AKIP in the plant comprises expression of an AKIP by an expression cassette in the plant, the expression cassette comprising a nucleic acid encoding the AKIP, the nucleic acid operably linked to a heterologous plant developmental stage-specific and/or cell type-specific promoter, wherein expression of the AKIP promotes senescence.

24. The method of claim 23 wherein the plant is a potato plant.

25. The method of claim 23 wherein the plant is a cotton plant.

26. The method of claim 23 wherein the plant is a tobacco plant.

27. The method of claim 23 wherein the plant is selected from the group consisting of alfalfa, apple, apricot, asparagus, avocado, banana, barley, bean, blackberry, broccoli, cabbage, carrot, cauliflower, celery, cherry, chicory, clover, cucumber, eggplant, garlic, grape, hemp, lettuce, maize, mango, melon, miscanthus, nectarine, onion, papaya, pea, peach, pear, pepper, pineapple, plum, pumpkin, quince, radish, rapeseed, rice, raspberry, rye, soybean, sorghum, spinach, squash, strawberry, sugarcane, sugarbeet, sunflower, sweet potato, switchgrass, tomato, turnip, wheat, zucchini, aster, basil, bay leaf, begonia, chives, chrysanthemum, cilantro, clover, delphinium, dill, eucalyptus, lavender, lemon grass, mint, oregano, parsley, rosemary, savory, sunflower, tarragon, thyme, and zinnia.

28. The method of claim 23 wherein expression of the AKIP promotes senescence manifested by drying of the plant or portion of the plant.

29. The method of claim 23 wherein expression of the AKIP promotes senescence manifested by defoliation of the plant.


SEQUENCE LISTING (96 sequences)

SEQ ID NO: 1 - DNA, 1577 bp, Solanum tuberosum
ccaaatcagaaaaccctaatttcaactatttctctctctatacctcaatattcccatggcgaagaagcgaaaaaccagagcttccgaaccttcacaaccagttgaagaaccagtagagattaagcaagaacctgaggttgaaaccgcacaagaagaagaatacgagaacgatgccgtagaagtagaggaagaagaagcagtacatgaggacgaagaagaacaagatcccgaagaggaagacgaagaaggcggtgacgatgaggaggaagaagaagaaatgcccggatctggaaatgacgctgtagaggatgataacgagaagtcggttaatgaacaggaacaagttaatggaggccctgaggtgaagatggaggaaggtaatgaagaggatttggaagatgagccacttgagaatctgttagagccatttacgaaggatcaacttacagcgcttattaaagaagctttagctaaataccctgatttcaaagaaaatattcagaaattggctgataaagatccggcacaccggaaaatctttgtccacggtttaggttgggatacgacagctgaaacgctaactagtgtttttgcaacttacggtgaaattgaggattgtaaagctgttacagataaggtttcggggaaatcgaaaggttatggatttatattgtttaagcataggagtggtgctaggaaagctttgaaagaaccacagaagaagattggtacaaggatgacttcttgccagttggcttctgctgggccggttccggctcctccacctactacagcagccccaccggtttcggagtacacacagaggaaaatttttgttagcaatgtagctgctgatcttgaacctcaaaagttgttggagtacttctcaaagtttggtgaagttgaggagggtccacttggattggataagcaaaatgggaaacctaaggggttttgtttgtttgtgtataagactgttgagggtgctaggaaggcattggaggagccacataagacctttgaagggcatacccttcattgtcagaaggcgattgatggaccgaagcatagtaagaatttccatcagcagcaacagcctcagtcacaacagcattaccctcagcatcatcatcataatcagcagcagcattattatcagcatcctgcaaagaaggggaaatattcaggtagcagtgcaggagcaggatcagctgggcatttaatggcaccatcggggcctgcacctgtgggatacaatcctgctgtggcagctgtgaccccagctcttggacaggcactgacagccctgttagctacacaaggagctggtttggggattgggaatttgcttggaggttttggtgggccagtgaatcatcagggtgtgcaaccagtggtgaacaatgcaacaggatatggtgcccagggtggatatggagctcagcctcatatgcaataccaaaaccctcagatgcagggtggtgcaagaccacaaggtggtggtgccccatactctggctatggaggtcactaggaaaacttaaggtatctgtaatgctatggctctacttccaagt

SEQ ID NO: 2 - protein, 492 aa, Solanum tuberosum
MAKKRKTRASEPSQPVEEPVEIKQEPEVETAQEEEYENDAVEVEEEEAVHEDEEEQDPEEEDEEGGDDEEEEEEMPGSGNDAVEDDNEKSVNEQEQVNGGPEVKMEEGNEEDLEDEPLENLLEPFTKDQLTALIKEALAKYPDFKENIQKLADKDPAHRKIFVHGLGWDTTAETLTSVFATYGEIEDCKAVTDKVSGKSKGYGFILFKHRSGARKALKEPQKKIGTRMTSCQLASAGPVPAPPPTTAAPPVSEYTQRKIFVSNVAADLEPQKLLEYFSKFGEVEEGPLGLDKQNGKPKGFCLFVYKTVEGARKALEEPHKTFEGHTLHCQKAIDGPKHSKNFHQQQQPQSQQHYPQHHHHNQQQHYYQHPAKKGKYSGSSAGAGSAGHLMAPSGPAPVGYNPAVAAVTPALGQALTALLATQGAGLGIGNLLGGFGGPVNHQGVQPVVNNATGYGAQGGYGAQPHMQYQNPQMQGGARPQGGGAPYSGYGGH

SEQ ID NO: 3 - DNA, 1349 bp, Solanum tuberosum
tcttctctgagcgaaaatggatctaacaaagaaacgcaaagctgatgagaacggcggtgcttatcccgtatccaacgaactcgtcacaactgccgccgcgccgccggcacttgctgttctttccccggaagacatcgacaaaattcttgaaacattcaccaaagaccagtgcgtttcaattctccgtaacgccgccctacgctatgctgatgttctcgaaggtctacgtgctgtcgctgacgccgacgtatctaaccgcaagctcttcgttcgtggtcttggttgggagaccaccaccgacaaactccgacaggttttttctgagtttggagaactcgatgaggctgtagttataactgataaggcgtcttccagatctaaagggtatggttttgtaactttcaagcacgttgatgctgctattctttctttaaaggtgcccaacaagataattgatggccgtgttaccgtcacacagcttgctgctgcgggtaattctgggaattctcagtcttctgatgttgcgcttaggaagatctatgttggtaatgttccatttgaaatttcgtctgagaagcttttgaatcacttttctatgtatggagagattgaagaggggcctttgggatttgataaacaaactggaaaagctaaaggttttgctttttttgtctacaagactgaggacggagctagggcatctttagttgatcccgtgaagacagtagaaggacatcaggtcttgtgtaaattggcaaccgataataagaaggggaagccccagaatatggggcatgggggtgcccctggaatgaatgctggacctggaggaatgcctggtgatgatagggttggatccatgcccggttcaaattatggtgttcctgttagcggtttgggtccatattcagggttttcaggtgggcctggtccaggaatgcagcagccaccacctcagcctggcatggtggcacatcagaatccacatctgaattcagctattggcgggcctggatatggaaaccaaggaccaggatcctttacaggtggtgctggtggatatgctggcgctggtggatatgctggggctggtgggtatggtggtagttctggggattacagtgggtacaggatgcctcaaagctctgctggaatgcctagttcaggaggttatccagatggtggtaattatggtttgcagtcttcttatccctcacaggtaccacaaccaggagctgggtcaagggttccaccaggtgggatgtaccagggcatgcctccatactactgaggtgtcttgagagcttcttggatgcaacatgctttaaatggttgtggtgatttcctga

SEQ ID NO: 4 - protein, 424 aa, Solanum tuberosum
MDLTKKRKADENGGAYPVSNELVTTAAAPPALAVLSPEDIDKILETFTKDQCVSILRNAALRYADVLEGLRAVADADVSNRKLFVRGLGWETTTDKLRQVFSEFGELDEAVVITDKASSRSKGYGFVTFKHVDAAILSLKVPNKIIDGRVTVTQLAAAGNSGNSQSSDVALRKIYVGNVPFEISSEKLLNHFSMYGEIEEGPLGFDKQTGKAKGFAFFVYKTEDGARASLVDPVKTVEGHQVLCKLATDNKKGKPQNMGHGGAPGMNAGPGGMPGDDRVGSMPGSNYGVPVSGLGPYSGFSGGPGPGMQQPPPQPGMVAHQNPHLNSAIGGPGYGNQGPGSFTGGAGGYAGAGGYAGAGGYGGSSGDYSGYRMPQSSAGMPSSGGYPDGGNYGLQSSYPSQVPQPGAGSRVPPGGMYQGMPPYY

SEQ ID NO: 5 - DNA, 1437 bp, Arabidopsis thaliana
atgacaaagaagagaaagctcgaaggagaagaatctaacgaagctgaagaaccgtcgcagaagctgaagcagacgccggaggaggagcagcagcttgtaattaaaaatcaggataatcaaggagacgttgaagaagttgaatacgaggaagtagaggaagagcaagaagaagaagtagaagatgatgacgatgaagatgatggtgatgagaatgaggatcagacggatgggaatcgtatagaggcggcggcgacgtctggatctggcaatcaagaagatgatgacgatgaaccaattcaggatctgttggaaccattttcaaaggagcaagttttgagtcttcttaaggaagcagcggagaagcatgttgatgtagccaatcgaattagggaagtagctgatgaggaccctgttcatcggaagatctttgtgcacggtcttggttgggatacgaaaacagagacgcttattgaagcttttaagcagtacggagagatcgaagattgcaaggctgtgtttgataagatctcagggaaatctaaaggttatggctttattctgtacaagtctcgttcaggtgctcgcaacgcactcaagcagcctcagaagaagattggtagtcgtatgacggcatgccaattggcttctaaaggacctgtctttggtggagctcctatcgctgctgcagctgtttcagcgcctgctcagcattcgaactcggagcatactcagaaaaagatttatgttagtaatgtcggagcagagcttgatccacagaagttgctgatgtttttctcaaagtttggagaaattgaagaaggtcctttgggtcttgataagtatactgggagacctaaaggtttctgtctctttgtttataagtcgtctgaaagcgccaagagggctttggaggagccacacaagacttttgaaggccatatcttgcattgccagaaagctatcgatggtcccaaaccgggcaagcagcagcagcatcaccataacccacacgcctataacaatcctcgttaccaaaggaatgataacaatggttatggtccgcctggaggtcatggacatctcatggctggtaacccagctgggatgggtggtccaacggcacaggtgataaacccggccattggacaggccttgacagctttattggcatctcagggagctggtttggcttttaatccggcaattggacaggctttgttgggttccttggggacagctgctggtgtaaacccagggaatggagttggaatgccaactggttacggtactcaagctatggcaccggggacaatgcctgggtacggtacacaacctgggttgcaaggcggttatcagactccgcaacctggtcaaggcggtacaagtagagggcaacatggtgtcgggccatacggtactccttacatgggtcactaa

SEQ ID NO: 6 - protein, 478 aa, Arabidopsis thaliana
MTKKRKLEGEESNEAEEPSQKLKQTPEEEQQLVIKNQDNQGDVEEVEYEEVEEEQEEEVEDDDDEDDGDENEDQTDGNRIEAAATSGSGNQEDDDDEPIQDLLEPFSKEQVLSLLKEAAEKHVDVANRIREVADEDPVHRKIFVHGLGWDTKTETLIEAFKQYGEIEDCKAVFDKISGKSKGYGFILYKSRSGARNALKQPQKKIGSRMTACQLASKGPVFGGAPIAAAAVSAPAQHSNSEHTQKKIYVSNVGAELDPQKLLMFFSKFGEIEEGPLGLDKYTGRPKGFCLFVYKSSESAKRALEEPHKTFEGHILHCQKAIDGPKPGKQQQHHHNPHAYNNPRYQRNDNNGYGPPGGHGHLMAGNPAGMGGPTAQVINPAIGQALTALLASQGAGLAFNPAIGQALLGSLGTAAGVNPGNGVGMPTGYGTQAMAPGTMPGYGTQPGLQGGYQTPQPGQGGTSRGQHGVGPYGTPYMGH

SEQ ID NO: 7 - DNA, 1356 bp, Arabidopsis thaliana
atgacaaagaagagaaagctcgaatctgaatccaacgaaacgtcagagccgacggagaagcagcagcagcaatgtgaaaaagaggatccggaaatcagaaatgttgataatcaaagagacgacgacgaacaagtagtagagcaagacacactaaaggagatgcacgaagaggaagctaaaggtgaagataacatagaagcggagacgtcgtccggatctgggaatcaaggaaatgaggatgatgacgaagaagaacctattgaggatctattggaaccgttttcaaaggatcaacttttgattcttctcaaggaagctgcagagagacatcgcgatgtagctaatcgaatccggattgtggcggatgaagatcttgttcatcgtaagatcttcgttcacgggcttggatgggatactaaagctgattcacttatcgacgcttttaaacagtacggagagattgaagattgcaaatgtgtggttgataaggtatctgggcaatctaaaggttatggctttatcctctttaagtcaaggtctggtgctcgtaacgctcttaagcagcctcagaagaagattggaactcgtatgactgcgtgccagctggcgtctataggacctgttcaggggaaccctgttgtggctcctgctcagcatttcaatcctgagaatgttcagaggaagatttatgtcagtaacgttagtgcagacattgatccgcagaagttgctggagtttttctcaaggtttggggagatagaagaaggtcctttggggcttgataaagctactgggagacctaaaggtttcgctttgtttgtctatagatccctagaaagtgccaagaaggcattggaggagccacacaagacttttgaaggccatgtcttgcattgccacaaagcaaatgatgggccaaaacaggttaagcaacatcaacataaccataactctcacaatcaaaattcccgttaccaaaggaacgacaacaatggttatggtgcccctggaggccatggacatttcatagctggtaataaccaagctgtgcaggcgtttaatccggccattggccaggccctcacagctttgctggcatctcagggtgctgggttgggtttaaaccaagcatttgggcaggctttgttggggacattagggacagctagcccaggagctgtaggtggaatgccaagtggctatggtactcaagcaaatatctcacctggggtctatcctgggtacggtgctcaagccgggtaccagggcggttatcagactcagcaacctggtcagggcggtgcgggaagagggcagcatggtgctgggtatggtggtccttacatgggtcgttag

SEQ ID NO: 8 - protein, 451 aa, Arabidopsis thaliana
MTKKRKLESESNETSEPTEKQQQQCEKEDPEIRNVDNQRDDDEQVVEQDTLKEMHEEEAKGEDNIEAETSSGSGNQGNEDDDEEEPIEDLLEPFSKDQLLILLKEAAERHRDVANRIRIVADEDLVHRKIFVHGLGWDTKADSLIDAFKQYGEIEDCKCVVDKVSGQSKGYGFILFKSRSGARNALKQPQKKIGTRMTACQLASIGPVQGNPVVAPAQHFNPENVQRKIYVSNVSADIDPQKLLEFFSRFGEIEEGPLGLDKATGRPKGFALFVYRSLESAKKALEEPHKTFEGHVLHCHKANDGPKQVKQHQHNHNSHNQNSRYQRNDNNGYGAPGGHGHFIAGNNQAVQAFNPAIGQALTALLASQGAGLGLNQAFGQALLGTLGTASPGAVGGMPSGYGTQANISPGVYPGYGAQAGYQGGYQTQQPGQGGAGRGQHGAGYGGPYMGR

SEQ ID NO: 9 - DNA, 1215 bp, Arabidopsis thaliana
atggatatgatgaagaagcgtaagctcgatgagaacggtaacggactcaataccaacggtggcggaaccattggtccaactcgattgtcgccgcaagacgcgaggaagattatcgagcgattcaccactgatcagctgctcgacttattgcaagaggctatcgtacgccatcccgacgtgcttgaatctgtccgattaaccgccgactctgatatctcgcagcggaagctattcatccgtggtctcgcggcggatactactacggaaggtctccgttcgcttttttccagttacggagatctagaagaagctattgtgatccttgacaaggtcactgggaaatcgaaagggtatggtttcgtcacttttatgcacgttgatggagctttactagctctgaaagaaccatctaagaaaattgatgggcgtgttacagtaacgcagctcgcggcatctggaaatcaaggtacgggttcccagattgcagatatatcaatgaggaagatctatgtggcgaatgttccttttgatatgccggcggataggctattgaatcactttatggcttatggagatgttgaggaaggtcctttgggttttgataaggttactggtaaatctagaggctttgcattgtttgtctacaagacagcggaaggtgcgcaggctgctcttgctgatccggtgaaggtgattgatggaaaacatttgaattgtaagttggctgttgatggtaagaaaggagggaagcctggaatgccacaagctcaagatggtggttctggtcatggacacgttcatggtgagggaatgggaatggtgcgtcctgcaggaccttatggtgcagctggtggtatcagtgcttatggtgggtactctggaggaccaccagcgcatcacatgaattccacacactcctcaatgggtgttggttctgctgggtatggtggacactatggtggatatggaggtccaggtggtactggcgtctacggtggacttggcggtgggtatggtgggcctggtactggaagtgggcagtatagaatgccaccaagttcgatgcctggtggtggtggttatcccgagagtgggcattacggtctttcatcatctgctgggtatcctggacagcatcatcaggctgttggaacgtcccctgtgccaagagttcctcatggcggaatgtaccccaacggtccaccaaactactga

SEQ ID NO: 10 - protein, 404 aa, Arabidopsis thaliana
MDMMKKRKLDENGNGLNTNGGGTIGPTRLSPQDARKIIERFTTDQLLDLLQEAIVRHPDVLESVRLTADSDISQRKLFIRGLAADTTTEGLRSLFSSYGDLEEAIVILDKVTGKSKGYGFVTFMHVDGALLALKEPSKKIDGRVTVTQLAASGNQGTGSQIADISMRKIYVANVPFDMPADRLLNHFMAYGDVEEGPLGFDKVTGKSRGFALFVYKTAEGAQAALADPVKVIDGKHLNCKLAVDGKKGGKPGMPQAQDGGSGHGHVHGEGMGMVRPAGPYGAAGGISAYGGYSGGPPAHHMNSTHSSMGVGSAGYGGHYGGYGGPGGTGVYGGLGGGYGGPGTGSGQYRMPPSSMPGGGGYPESGHYGLSSSAGYPGQHHQAVGTSPVPRVPHGGMYPNGPPNY

SEQ ID NO: 11 - DNA, 2188 bp, Arabidopsis thaliana
gatatctctttttatattcaaacaataagttgagatatgtttgagaagaggacaactattctcgtggagcaccgagtttgttttatattagaaacccgattgttatttttagactgagacaaaaaagtaaaatcgttgattgttaaaatttaaaattagtttcattacgtttcgataaaaaaatgattagtttatcatagcttaattatagcattgatttctaaatttgttttttgaccacccttttttctctctttggtgttttcttaacattagaagaacccataacaatgtacgttcaaattaattaaaaacaatatttccaagttttatatacgaaacttgttttttttaatgaaaacagttgaatagttgattatgaattagttagatcaatactcaatatatgatcaatgatgtatatatatgaactcagttgttatacaagaaatgaaaatgctatttaaataccgatcatgaagtgttaaaaagtgtcagaatatgacatgaagcgttttgtcctaccgggtattcgagttataggtttggatctctcaagaatattttgggccatattagttatatttgggcttaagcgttttgcaaagagacgaggaagaaagattgggtcaagttaacaaaacagagacactcgtattagttggtactttggtagcaagtcgatttatttgccagtaaaaacttggtacacaactgacaactcgtatcgttattagtttgtacttggtacctttggttcaagaaaaagttgatatagttaaatcagttgtgttcatgaggtgattgtgatttaatttgttgactagggcgattccttcacatcacaataacaaagttttatagattttttttttataacatttttgccacgcttcgtaaagtttggtatttacaccgcatttttccctgtacaagaattcatatattatttatttatatactccagttgacaattataagtttataacgtttttacaattatttaaataccatgtgaagatccaagaatatgtcttacttcttctttgtgtaagaaaactaactatatcactataataaaataattctaatcattatatttgtaaatatgcagttatttgtcaattttgaatttagtattttagacgttatcacttcagccaaatatgatttggatttaagtccaaaatgcaatttcgtacgtatccctcttgtcgtctaatgattatttcaatatttcttatattatccctaactacagagctacatttatattgtattctaatgacagggaaactttcatagagattcagatagatgaaattggtgggaaacatcattgaacaggaaacttttagcaaatcatatcgatttatctacaaaagaatacttagcgtaatgaagttcacttgttgtgaatgactatgatttgatcaaattagttaattttgtcgaatcatttttctttttgatttgattaagcttttaacttgcacgaatggttctcttgtgaataaacagaatctttgaattcaaactatttgattagtgaaaagacaaaagaagattccttgtttttatgtgattagtgattttgatgcatgaaaggtacctacgtactacaagaaaaataaacatgtacgtaactacgtatcagcatgtaaaagtatttttttccaaataatttatactcatgatagattttttttttttgaaatgtcaattaaaaatgctttcttaaatattaattttaattaattaaataaggaaatatatttatgcaaaacatcatcaacacatatccaacttcgaaaatctctatagtacacaagtagagaaattaaattttactagatacaaacttcctaatcatcaaatataaatgtttacaaaactaattaaacccaccactaaaattaactaaaaatccgagcaaagtgagtgaacaagacttgatttcaggttgatgtaggactaaaatggctacgtatcaaacatcaacgatcatttagttatgtatgaatgaatgtagtcattacttgtaaaacaaaaatgctttgatttggatcaatcacttcatgtgaacattagcaattacatcaaccttattttcactataaaaccccatctcagtacccttctgaagtaatcaaattaagagcaaaagtcatttaactttcctaaaaca

SEQ ID NO: 12 - DNA, 975 bp, Ipomoea batatas
atcactttacaaaccaacaaatggtgcatcatctcaaatgtcattgtcaaacctgtcaggacaaattataaagtacttgatcacaaatacaattacacatggattttaaatagtcggaccgctgtccaaacaaatgacaatgatgacacgccatttgttccatgtaagtggcttatttgtttctacatactttaattgtagtttagttatttagaatttcgttatgttttattaaaatatataagtatcgagattatttctttgatttttattaatttaatattttttccctctcattattatatggttattttaatcaattaaggaacactaatttaaaaatacgcattagacattgatttaatctcgcaacgcgcgtgtgataattagtagttactacaaatgcaatagcattagcagtttggcaccattaattaacggctaatgtacaaaattagagtcctcccctaccaattcaaagcaacgtctacggaattcgaacttcattgtcgctatcatttgtaagataaattaggagaaacaatattttgtcgacatagagaataactgaatatgattcatttatttaattcaattaatataactatttaaataaaactatttttttttcaaatttatctcactgtttattaatataaaataagtttaggaggaaaatttaggattactctaatacggagtattaattattgattggccgttaattaacggataacgcacaacattagagcctagctttatcaaagcagcgtctacggaattcaaaacttcatcgtcgctatcatttgtaacttgccggcttttccattgaatctacctccgtgataagattctgtgacaatccagccctataaataccgttgcatctttaatctaatgcattcactcacaacactttacagttgtaaacattttacaaccatttttaattaaggtttcaggttgtcaattact

SEQ ID NO: 13 - DNA, 1163 bp, Arabidopsis thaliana
atggttgcaacagagaggatgaatttataagttttcaacaccgcttttcttattagacggacaacaatctatagtggagtaaatttttatttttggtaaaatggttagtgaattcaaatatctaaattttgtgactcactaacattaacaaatatgcataagacataaaaaaaagaaagaataattcttatgaaacaagaaaaaaaacctatacaatcaatctttaggaattgacgatgtagaattgtagatgataaattttctcaaatatagatgggcctaatgaagggtgccgcttattggatctgacccattttgaggacattaatattttcattggttataagccttttaatcaaaattgtcattaaattgatgtctccctctcgggtcattttcctttctccctcacaattaatgtagactttagcaatttgcacgctgtgctttgtctttatatttagtaacacaaacattttgacttgtcttgtagagtttttctcttttatttttctatccaatatgaaaactaaaagtgttctcgtatacatatattaaaattaaagaaacctatgaaaacaccaatacaaatgcgatattgttttcagttcgacgtttcatgtttgttagaaaatttctaatgacgtttgtataaaatagacaattaaacgccaaacactacatctgtgttttcgaacaatattgcgtctgcgtttccttcatctatctctctcagtgtcacaatgtctgaactaagagacagctgtaaactatcattaagacataaactaccaaagtatcaagctaatgtaaaaattactctcatttccacgtaacaaattgagttagcttaagatattagtgaaactaggtttgaattttcttcttcttcttccatgcatcctccgaaaaaagggaaccaatcaaaactgtttgcatatcaaactccaacactttacagcaaatgcaatctataatctgtgatttatccaataaaaacctgtgatttatgtttggctccagcgatgaaagtctatgcatgtgatctctatccaacatgagtaattgttcagaaaataaaaagtagctgaaatgtatctatataaagaatcatccacaagtactattttcacacactacttcaaaatcactactcaagaaat

SEQ ID NO: 14 - DNA, 9192 bp, artificial
  pORE-R2 plasmid encoding E. coli beta- gluronidase operably linked to a cauliflower mosaic virus 35S ribosomal promoter
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaaggccttggatccacccgggagaattcgtcgactttgcggccgcatcgatactgcaggagctcatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggctattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatattaccgtggtgacgcatgtcgcgcaagactgtaaccacgcttctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatttacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacccgtaaactcgacccgacccgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaggtaccttttactagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcgggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 15 - DNA, 9041 bp, artificial
  pORE plasmid encoding potato AKIP1/2 operably linked to a cauliflower mosaic virus 35S ribosomal promoter
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaaggccttggatccacccgggagaattcgtcgactttgcggccgcatcgatactgcaggagctcggatccactagtaacggccgccagtgtgctggaattcgcccttccaaatcagaaaaccctaatttcaactatttctctctctatacctcaatattcccatggcgaagaagcgaaaaaccagagcttccgaaccttcacaaccagttgaagaaccagtagagattaagcaagaacctgaggttgaaaccgcacaagaagaagaatacgagaacgatgccgtagaagtagaggaagaagaagcagtacatgaggacgaagaagaacaagatcccgaagaggaagacgaagaaggcggtgacgatgaggaggaagaagaagaaatgcccggatctggaaatgacgctgtagaggatgataacgagaagtcggttaatgaacaggaacaagttaatggaggccctgaggtgaagatggaggaaggtaatgaagaggatttggaagatgagccacttgagaatctgttagagccatttacgaaggatcaacttacagcgcttattaaagaagctttagctaaataccctgatttcaaagaaaatattcagaaattggctgataaagatccggcacaccggaaaatctttgtccacggtttaggttgggatacgacagctgaaacgctaactagtgtttttgcaacttacggtgaaattgaggattgtaaagctgttacagataaggtttcggggaaatcgaaaggttatggatttatattgtttaagcataggagtggtgctaggaaagctttgaaagaaccacagaagaagattggtacaaggatgacttcttgccagttggcttctgctgggccggttccggctcctccacctactacagcagccccaccggtttcggagtacacacagaggaaaatttttgttagcaatgtagctgctgatcttgaacctcaaaagttgttggagtacttctcaaagtttggtgaagttgaggagggtccacttggattggataagcaaaatgggaaacctaaggggttttgtttgtttgtgtataagactgttgagggtgctaggaaggcattggaggagccacataagacctttgaagggcatacccttcattgtcagaaggcgattgatggaccgaagcatagtaagaatttccatcagcagcaacagcctcagtcacaacagcattaccctcagcatcatcatcataatcagcagcagcattattatcagcatcctgcaaagaaggggaaatattcaggtagcagtgcaggagcaggatcagctgggcatttaatggcaccatcggggcctgcacctgtgggatacaatcctgctgtggcagctgtgaccccagctcttggacaggcactgacagccctgttagctacacaaggagctggtttggggattgggaatttgcttggaggttttggtgggccagtgaatcatcagggtgtgcaaccagtggtgaacaatgcaacaggatatggtgcccagggtggatatggagctcagcctcatatgcaataccaaaaccctcagatgcagggtggtgcaagaccacaaggtggtggtgccccatactctggctatggaggtcactaggaaaacttaaggtatctgtaatgctatggctctacttccaagtaagggcgaattctgcagatatccatcacactggcggccgctcgagcatgcatctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcgggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 16 - DNA, 8813 bp, artificial
  pORE plasmid encoding potato AKIP3 operably linked to a 35S ribosomal promoter
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcggaaagcttgcatgcctgcaggtccccagattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaaggccttggatccacccgggagaattcgtcgactttgcggccgcatcgatactgcaggagctcggatccactagtaacggccgccagtgtgctggaattcgccctttcttctctgagcgaaaatggatctaacaaagaaacgcaaagctgatgagaacggcggtgcttatcccgtatccaacgaactcgtcacaactgccgccgcgccgccggcacttgctgttctttccccggaagacatcgacaaaattcttgaaacattcaccaaagaccagtgcgtttcaattctccgtaacgccgccctacgctatgctgatgttctcgaaggtctacgtgctgtcgctgacgccgacgtatctaaccgcaagctcttcgttcgtggtcttggttgggagaccaccaccgacaaactccgacaggttttttctgagtttggagaactcgatgaggctgtagttataactgataaggcgtcttccagatctaaagggtatggttttgtaactttcaagcacgttgatgctgctattctttctttaaaggtgcccaacaagataattgatggccgtgttaccgtcacacagcttgctgctgcgggtaattctgggaattctcagtcttctgatgttgcgcttaggaagatctatgttggtaatgttccatttgaaatttcgtctgagaagcttttgaatcacttttctatgtatggagagattgaagaggggcctttgggatttgataaacaaactggaaaagctaaaggttttgctttttttgtctacaagactgaggacggagctagggcatctttagttgatcccgtgaagacagtagaaggacatcaggtcttgtgtaaattggcaaccgataataagaaggggaagccccagaatatggggcatgggggtgcccctggaatgaatgctggacctggaggaatgcctggtgatgatagggttggatccatgcccggttcaaattatggtgttcctgttagcggtttgggtccatattcagggttttcaggtgggcctggtccaggaatgcagcagccaccacctcagcctggcatggtggcacatcagaatccacatctgaattcagctattggcgggcctggatatggaaaccaaggaccaggatcctttacaggtggtgctggtggatatgctggcgctggtggatatgctggggctggtgggtatggtggtagttctggggattacagtgggtacaggatgcctcaaagctctgctggaatgcctagttcaggaggttatccagatggtggtaattatggtttgcagtcttcttatccctcacaggtaccacaaccaggagctgggtcaagggttccaccaggtgggatgtaccagggcatgcctccatactactgaggtgtcttgagagcttcttggatgcaacatgctttaaatggttgtggtgatttcctgaaagggcgaattctgcagatatccatcacactggcggccgctcgagcatgcatctagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcgggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 17 - DNA, 10130 bp, artificial
  pORE_R2 plasmid encoding Arabidopsis thaliana AKIP2 operably linked to a senescence-activated promoter, pSAG12
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggatatctctttttatattcaaacaataagttgagatatgtttgagaagaggacaactattctcgtggagcaccgagtttgttttatattagaaacccgattgttatttttagactgagacaaaaaagtaaaatcgttgattgttaaaatttaaaattagtttcattacgtttcgataaaaaaatgattagtttatcatagcttaattatagcattgatttctaaatttgttttttgaccacccttttttctctctttggtgttttcttaacattagaagaacccataacaatgtacgttcaaattaattaaaaacaatatttccaagttttatatacgaaacttgttttttttaatgaaaacagttgaatagttgattatgaattagttagatcaatactcaatatatgatcaatgatgtatatatatgaactcagttgttatacaagaaatgaaaatgctatttaaataccgatcatgaagtgttaaaaagtgtcagaatatgacatgaagcgttttgtcctaccgggtattcgagttataggtttggatctctcaagaatattttgggccatattagttatatttgggcttaagcgttttgcaaagagacgaggaagaaagattgggtcaagttaacaaaacagagacactcgtattagttggtactttggtagcaagtcgatttatttgccagtaaaaacttggtacacaactgacaactcgtatcgttattagtttgtacttggtacctttggttcaagaaaaagttgatatagttaaatcagttgtgttcatgaggtgattgtgatttaatttgttgactagggcgattccttcacatcacaataacaaagttttatagattttttttttataacatttttgccacgcttcgtaaagtttggtatttacaccgcatttttccctgtacaagaattcatatattatttatttatatactccagttgacaattataagtttataacgtttttacaattatttaaataccatgtgaagatccaagaatatgtcttacttcttctttgtgtaagaaaactaactatatcactataataaaataattctaatcattatatttgtaaatatgcagttatttgtcaattttgaatttagtattttagacgttatcacttcagccaaatatgatttggatttaagtccaaaatgcaatttcgtacgtatccctcttgtcgtctaatgattatttcaatatttcttatattatccctaactacagagctacatttatattgtattctaatgacagggaaactttcatagagattcagatagatgaaattggtgggaaacatcattgaacaggaaacttttagcaaatcatatcgatttatctacaaaagaatacttagcgtaatgaagttcacttgttgtgaatgactatgatttgatcaaattagttaattttgtcgaatcatttttctttttgatttgattaagcttttaacttgcacgaatggttctcttgtgaataaacagaatctttgaattcaaactatttgattagtgaaaagacaaaagaagattccttgtttttatgtgattagtgattttgatgcatgaaaggtacctacgtactacaagaaaaataaacatgtacgtaactacgtatcagcatgtaaaagtatttttttccaaataatttatactcatgatagattttttttttttgaaatgtcaattaaaaatgctttcttaaatattaattttaattaattaaataaggaaatatatttatgcaaaacatcatcaacacatatccaacttcgaaaatctctatagtacacaagtagagaaattaaattttactagatacaaacttcctaatcatcaaatataaatgtttacaaaactaattaaacccaccactaaaattaactaaaaatccgagcaaagtgagtgaacaagacttgatttcaggttgatgtaggactaaaatggctacgtatcaaacatcaacgatcatttagttatgtatgaatgaatgtagtcattacttgtaaaacaaaaatgctttgatttggatcaatcacttcatgtgaacattagcaattacatcaaccttattttcactataaaaccccatctcagtacccttctgaagtaatcaaattaagagcaaaagtcatttaactttcctaaaacactcgaggtatttttacaacaattaccaacaacaacaaacaacaaacaacattacaattactatttacaattacaattaccatggactacaaggacgacgatgacaagcatatgacaaagaagagaaagctcgaatctgaatccaacgaaacgtcagagccgacggagaagcagcagcagcaatgtgaaaaagaggatccggaaatcagaaatgttgataatcaaagagacgacgacgaacaagtagtagagcaagacacactaaaggagatgcacgaagaggaagctaaaggtgaagataacatagaagcggagacgtcgtccggatctgggaatcaaggaaatgaggatgatgacgaagaagaacctattgaggatctattggaaccgttttcaaaggatcaacttttgattcttctcaaggaagctgcagagagacatcgcgatgtagctaatcgaatccggattgtggcggatgaagatcttgttcatcgtaagatcttcgttcacgggcttggatgggatactaaagctgattcacttatcgacgcttttaaacagtacggagagattgaagattgcaaatgtgtggttgataaggtatctgggcaatctaaaggttatggctttatcctctttaagtcaaggtctggtgctcgtaacgctcttaagcagcctcagaagaagattggaactcgtatgactgcgtgccagctggcgtctataggacctgttcaggggaaccctgttgtggctcctgctcagcatttcaatcctgagaatgttcagaggaagatttatgtcagtaacgttagtgcagacattgatccgcagaagttgctggagtttttctcaaggtttggggagatagaagaaggtcctttggggcttgataaagctactgggagacctaaaggtttcgctttgtttgtctatagatccctagaaagtgccaagaaggcattggaggagccacacaagacttttgaaggccatgtcttgcattgccacaaagcaaatgatgggccaaaacaggttaagcaacatcaacataaccataactctcacaatcaaaattcccgttaccaaaggaacgacaacaatggttatggtgcccctggaggccatggacatttcatagctggtaataaccaagctgtgcaggcgtttaatccggccattggccaggccctcacagctttgctggcatctcagggtgctgggttgggtttaaaccaagcatttgggcaggctttgttggggacattagggacagctagcccaggagctgtaggtggaatgccaagtggctatggtactcaagcaaatatctcacctggggtctatcctgggtacggtgctcaagccgggtaccagggcggttatcagactcagcaacctggtcagggcggtgcgggaagagggcagcatggtgctgggtatggtggtccttacatgggtcgttagattagccactcaggaagggcgaattcgtttaaacctgcaggactagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcgggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 18 - DNA, 10524 bp, artificial
  pORE_R2 plasmid encoding E. coli beta- glucuronidase operably linked to a senescence-activated promoter, pSAG12
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctagggaagttcctattccgaagttcctattctctgaaaagtataggaacttctttgcgtattgggcgctcttggcctttttggccaccggtcgtacggttaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccactagacgctcaccgggctggttgccctcgccgctgggctggcggccgtctatggccctgcaaacgcgccagaaacgccgtcgaagccgtgtgcgagacaccgcagccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctctcgcgttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatattggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaagccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactgagatcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggatctcctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgctagtggatctcccagtcacgacgttgtaaaacgggcgccccgcgggatatctctttttatattcaaacaataagttgagatatgtttgagaagaggacaactattctcgtggagcaccgagtttgttttatattagaaacccgattgttatttttagactgagacaaaaaagtaaaatcgttgattgttaaaatttaaaattagtttcattacgtttcgataaaaaaatgattagtttatcatagcttaattatagcattgatttctaaatttgttttttgaccacccttttttctctctttggtgttttcttaacattagaagaacccataacaatgtacgttcaaattaattaaaaacaatatttccaagttttatatacgaaacttgttttttttaatgaaaacagttgaatagttgattatgaattagttagatcaatactcaatatatgatcaatgatgtatatatatgaactcagttgttatacaagaaatgaaaatgctatttaaataccgatcatgaagtgttaaaaagtgtcagaatatgacatgaagcgttttgtcctaccgggtattcgagttataggtttggatctctcaagaatattttgggccatattagttatatttgggcttaagcgttttgcaaagagacgaggaagaaagattgggtcaagttaacaaaacagagacactcgtattagttggtactttggtagcaagtcgatttatttgccagtaaaaacttggtacacaactgacaactcgtatcgttattagtttgtacttggtacctttggttcaagaaaaagttgatatagttaaatcagttgtgttcatgaggtgattgtgatttaatttgttgactagggcgattccttcacatcacaataacaaagttttatagattttttttttataacatttttgccacgcttcgtaaagtttggtatttacaccgcatttttccctgtacaagaattcatatattatttatttatatactccagttgacaattataagtttataacgtttttacaattatttaaataccatgtgaagatccaagaatatgtcttacttcttctttgtgtaagaaaactaactatatcactataataaaataattctaatcattatatttgtaaatatgcagttatttgtcaattttgaatttagtattttagacgttatcacttcagccaaatatgatttggatttaagtccaaaatgcaatttcgtacgtatccctcttgtcgtctaatgattatttcaatatttcttatattatccctaactacagagctacatttatattgtattctaatgacagggaaactttcatagagattcagatagatgaaattggtgggaaacatcattgaacaggaaacttttagcaaatcatatcgatttatctacaaaagaatacttagcgtaatgaagttcacttgttgtgaatgactatgatttgatcaaattagttaattttgtcgaatcatttttctttttgatttgattaagcttttaacttgcacgaatggttctcttgtgaataaacagaatctttgaattcaaactatttgattagtgaaaagacaaaagaagattccttgtttttatgtgattagtgattttgatgcatgaaaggtacctacgtactacaagaaaaataaacatgtacgtaactacgtatcagcatgtaaaagtatttttttccaaataatttatactcatgatagattttttttttttgaaatgtcaattaaaaatgctttcttaaatattaattttaattaattaaataaggaaatatatttatgcaaaacatcatcaacacatatccaacttcgaaaatctctatagtacacaagtagagaaattaaattttactagatacaaacttcctaatcatcaaatataaatgtttacaaaactaattaaacccaccactaaaattaactaaaaatccgagcaaagtgagtgaacaagacttgatttcaggttgatgtaggactaaaatggctacgtatcaaacatcaacgatcatttagttatgtatgaatgaatgtagtcattacttgtaaaacaaaaatgctttgatttggatcaatcacttcatgtgaacattagcaattacatcaaccttattttcactataaaaccccatctcagtacccttctgaagtaatcaaattaagagcaaaagtcatttaactttcctaaaacactcgagtttttctagaaggccttggatccacccgggagaattcgtcgactttgcggccgcatcgatactgcaggagctcatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggctattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatattaccgtggtgacgcatgtcgcgcaagactgtaaccacgcttctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatttacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacccgtaaactcgacccgacccgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgaggtaccttttactagtgatatccctgtgtgaaattgttatccgctacgcgtgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcccatgggaagttcctattccgaagttcctattctctgaaaagtataggaacttcagcgatcgcagacgtcgggatcttctgcaagcatctctatttcctgaaggtctaacctcgaagatttaagatttaattacgtttataattacaaaattgattctagtatctttaatttaatgcttatacattattaattaatttagtactttcaatttgttttcagaaattattttactattttttataaaataaaagggagaaaatggctatttaaatactagcctattttatttcaattttagcttaaaatcagccccaattagccccaatttcaaattcaaatggtccagcccaattcctaaataacccacccctaacccgcccggtttccccttttgatccatgcagtcaacgcccagaatttccctatataattttttaattcccaaacacccctaactctatcccatttctcaccaaccgccacatagatctatcctcttatctctcaaactctctcgaaccttcccctaaccctagcagcctctcatcatcctcacctcaaaacccaccggggccggccatgattgaacaagatggattgcacgcaggttctccggccgcttgggtggagaggctattcggctatgactgggcacaacagacaatcggctgctctgatgccgccgtgttccggctgtcagcgcaggggaggccggttctttttgtcaagaccgacctgtccggtgccctgaatgaacttcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggcgcgcc

SEQ ID NO: 19 - DNA, 1066 bp, Arabidopsis thaliana
tcctaccattctcccatctttttaatttgtatgccttatacttaattttaattggttcaatcgtcatacttcatgaataagatgtttccttcaaatatataaacgatgcagtgtgtaatttgcagtcggtgtcggatttcgcacacattcgactcaacgaaattaatgttttgaaaatttaaattggacatcgaaaatttatattcattggatatatgtgtgtcccaaataaaagtaaacaagaatgtagaacacatcaaaataattaatatcacttatcctgcaacaaaatgagaaaacaacaaaattagatcataaaatatattattcaaaattatacttagtaaatttaatggtaaaagatcaaatatgacttgcttttcaaaatgttaaaacagtctcgaatacgtaaaataatttgatcacaaaataaagtatatagataacaagattttctaatctatatcaaattggaaaatattttttcataacagtgaaaagatttttttttaaaagtttgtacattgatttttgattcaaaaaccttagttaattaaaagatctcttaccaataaaagtttctgaatgggatttggtattgaactgtgtccttaaatccgggttttagttcaaccacgagtttaactacataaacgatttccggtcagcctggtcctgacgggccaccaattttcaaaacattgacgaaagcaagcccaataacatcaaatataattaacaaattttagtttgaatacggttactgttttggaaattgatacattccgatcttggactaatctataatccatgggctggctattttgcagtttcccgtaaataaaattatgaaaacatgattgatttctcgttggtagaccatgacatcacaggacacgtcaacattccaagtataaaggacaaaactgtccatttagattccaccacgtgtcccgataacgtttccgtctgtatctctctcttccgtcttctactcttcttcttcttcttcttcttccactcacaatctatcgagctaaaacactgaagcagacattagcgttga

SEQ ID NO: 20 - DNA, 2949 bp, artificial
  Sequence amplified by PCR from screening transgenic potato plants using primer sequences Forward 5′-AATCAGTTGTGTTCATGAGGTG-3′ SEQ ID No. 91; and Reverse 5′-AGCGGATAACAATTTCACACAGG-3′ SEQ ID No. 92
aatcagttgtgttcatgaggtgattgtgatttaatttgttgactagggcgattccttcacatcacaataacaaagttttatagattttttttttataacatttttgccacgcttcgtaaagtttggtatttacaccgcatttttccctgtacaagaattcatatattatttatttatatactccagttgacaattataagtttataacgtttttacaattatttaaataccatgtgaagatccaagaatatgtcttacttcttctttgtgtaagaaaactaactatatcactataataaaataattctaatcattatatttgtaaatatgcagttatttgtcaattttgaatttagtattttagacgttatcacttcagccaaatatgatttggatttaagtccaaaatgcaatttcgtacgtatccctcttgtcgtctaatgattatttcaatatttcttatattatccctaactacagagctacatttatattgtattctaatgacagggaaactttcatagagattcagatagatgaaattggtgggaaacatcattgaacaggaaacttttagcaaatcatatcgatttatctacaaaagaatacttagcgtaatgaagttcacttgttgtgaatgactatgatttgatcaaattagttaattttgtcgaatcatttttctttttgatttgattaagcttttaacttgcacgaatggttctcttgtgaataaacagaatctttgaattcaaactatttgattagtgaaaagacaaaagaagattccttgtttttatgtgattagtgattttgatgcatgaaaggtacctacgtactacaagaaaaataaacatgtacgtaactacgtatcagcatgtaaaagtatttttttccaaataatttatactcatgatagattttttttttttgaaatgtcaattaaaaatgctttcttaaatattaattttaattaattaaataaggaaatatatttatgcaaaacatcatcaacacatatccaacttcgaaaatctctatagtacacaagtagagaaattaaattttactagatacaaacttcctaatcatcaaatataaatgtttacaaaactaattaaacccaccactaaaattaactaaaaatccgagcaaagtgagtgaacaagacttgatttcaggttgatgtaggactaaaatggctacgtatcaaacatcaacgatcatttagttatgtatgaatgaatgtagtcattacttgtaaaacaaaaatgctttgatttggatcaatcacttcatgtgaacattagcaattacatcaaccttattttcactataaaaccccatctcagtacccttctgaagtaatcaaattaagagcaaaagtcatttaactttcctaaaacactcgaggtatttttacaacaattaccaacaacaacaaacaacaaacaacattacaattactatttacaattacaattaccatggactacaaggacgacgatgacaagcatatgacaaagaagagaaagctcgaatctgaatccaacgaaacgtcagagccgacggagaagcagcagcagcaatgtgaaaaagaggatccggaaatcagaaatgttgataatcaaagagacgacgacgaacaagtagtagagcaagacacactaaaggagatgcacgaagaggaagctaaaggtgaagataacatagaagcggagacgtcgtccggatctgggaatcaaggaaatgaggatgatgacgaagaagaacctattgaggatctattggaaccgttttcaaaggatcaacttttgattcttctcaaggaagctgcagagagacatcgcgatgtagctaatcgaatccggattgtggcggatgaagatcttgttcatcgtaagatcttcgttcacgggcttggatgggatactaaagctgattcacttatcgacgcttttaaacagtacggagagattgaagattgcaaatgtgtggttgataaggtatctgggcaatctaaaggttatggctttatcctctttaagtcaaggtctggtgctcgtaacgctcttaagcagcctcagaagaagattggaactcgtatgactgcgtgccagctggcgtctataggacctgttcaggggaaccctgttgtggctcctgctcagcatttcaatcctgagaatgttcagaggaagatttatgtcagtaacgttagtgcagacattgatccgcagaagttgctggagtttttctcaaggtttggggagatagaagaaggtcctttggggcttgataaagctactgggagacctaaaggtttcgctttgtttgtctatagatccctagaaagtgccaagaaggcattggaggagccacacaagacttttgaaggccatgtcttgcattgccacaaagcaaatgatgggccaaaacaggttaagcaacatcaacataaccataactctcacaatcaaaattcccgttaccaaaggaacgacaacaatggttatggtgcccctggaggccatggacatttcatagctggtaataaccaagctgtgcaggcgtttaatccggccattggccaggccctcacagctttgctggcatctcagggtgctgggttgggtttaaaccaagcatttgggcaggctttgttggggacattagggacagctagcccaggagctgtaggtggaatgccaagtggctatggtactcaagcaaatatctcacctggggtctatcctgggtacggtgctcaagccgggtaccagggcggttatcagactcagcaacctggtcagggcggtgcgggaagagggcagcatggtgctgggtatggtggtccttacatgggtcgttagattagccactcaggaagggcgaattcgtttaaacctgcaggactagtgatatccctgtgtgaaattgttatccgct

SEQ ID NO: 21 - protein, 60 aa, Arabidopsis thaliana
KIFVHGLGWDTKTETLIEAFKQYGEIEDCKAVFDKISGKSKGYGFILYKSRSGARNALKQ

SEQ ID NO: 22 - protein, 60 aa, Arabidopsis thaliana
KIFVHGLGWDTKADSLIDAFKQYGEIEDCKCVVDKVSGQSKGYGFILFKSRSGARNALKQ

SEQ ID NO: 23 - protein, 60 aa, Arabidopsis thaliana
KLFIRGLAADTTTEGLRSLFSSYGDLEEAIVILDKVTGKSKGYGFVTFMHVDGALLALKE

SEQ ID NO: 24 - protein, 60 aa, Solanum tuberosum
KIFVHGLGWDTTAETLTSVFATYGEIEDCKAVTDKVSGKSKGYGFILFKHRSGARKALKE

SEQ ID NO: 25 - protein, 60 aa, Solanum tuberosum
KLFVRGLGWETTTDKLRQVFSEFGELDEAVVITDKASSRSKGYGFVTFKHVDAAILSLKV

SEQ ID NO: 26 - protein, 74 aa, Arabidopsis thaliana
KIYVSNVGAELDPQKLLMFFSKFGEIEEGPLGLDKYTGRPKGFCLFVYKSSESAKRALEEPHKTFEGHILHCQK

SEQ ID NO: 27 - protein, 74 aa, Arabidopsis thaliana
KIYVSNVSADIDPQKLLEFFSRFGEIEEGPLGLDKATGRPKGFALFVYRSLESAKKALEEPHKTFEGHVLHCHK

SEQ ID NO: 28 - protein, 74 aa, Arabidopsis thaliana
KIYVANVPFDMPADRLLNHFMAYGDVEEGPLGFDKVTGKSRGFALFVYKTAEGAQAALADPVKVIDGKHLNCKL

SEQ ID NO: 29 - protein, 74 aa, Solanum tuberosum
KIFVSNVAADLEPQKLLEYFSKFGEVEEGPLGLDKQSGKPKGFCLFVYKTVEGARKALEEPHKTFEGHTLHCQK

SEQ ID NO: 30 - protein, 74 aa, Solanum tuberosum
KIYVGNVPFEISSEKLLNHFSMYGEIEEGPLGFDKQTGKAKGFAFFVYKTEDGARASLVDPVKTVEGHQVLCKL

SEQ ID NO: 31 - DNA, 27 bp, artificial
  primer for PCR amplification of At AKIP1
catatgacaaagaagagaaagctcgaa

SEQ ID NO: 32 - DNA, 28 bp, artificial
  primer for PCR amplification of At AKIP1
ggatacatgatacatttagtgacccatg

SEQ ID NO: 33 - DNA, 27 bp, artificial
  primer for PCR amplification of At AKIP2
catatgacaaagaagagaaagctcgaa

SEQ ID NO: 34 - DNA, 27 bp, artificial
  primer for PCR amplification of At AKIP2
cctgagtggctaatctaacgacccatg

SEQ ID NO: 35 - DNA, 28 bp, artificial
  primer for PCR amplification of At AKIP3
catatggatatgatgaagaagcgtaagc

SEQ ID NO: 36 - DNA, 27 bp, artificial
  primer for PCR amplification of At AKIP3
ttttcagtagtttggtggaccgttggg

SEQ ID NO: 37 - DNA, 24 bp, artificial
  primer for PCR amplification of At AKIP1
ctatcgctgctgcagctgtttcag

SEQ ID NO: 38 - DNA, 24 bp, artificial
  primer for PCR amplification of At AKIP1
gacttcatttagcatcagctcctt

SEQ ID NO: 39 - DNA, 22 bp, artificial
  primer for PCR amplification of At AKIP2
gggaaccctgttgtggctcctg

SEQ ID NO: 40 - DNA, 23 bp, artificial
  primer for PCR amplification of At AKIP2
ggtaccccatagatttttgttgg

SEQ ID NO: 41 - DNA, 24 bp, artificial
  primer for PCR amplification of At AKIP3
ctggtaaatctagaggctttgcat

SEQ ID NO: 42 - DNA, 24 bp, artificial
  primer for PCR amplification of At AKIP3
caatcaggtaatcaccactaagca

SEQ ID NO: 43 - DNA, 24 bp, artificial
  primer for PCR amplification of SAG12
gaccaatccaaaagcaacttctat

SEQ ID NO: 44 - DNA, 24 bp, artificial
  primer for PCR amplification of SAG12
tttagacatcaatcccacacaaac

SEQ ID NO: 45 - DNA, 24 bp, artificial
  primer for PCR amplification of SAG13
tgtaatggctacaaatctcgagtc

SEQ ID NO: 46 - DNA, 24 bp, artificial
  primer for PCR amplification of SAG13
ctagtctgccgtcaaattggtaat

SEQ ID NO: 47 - DNA, 24 bp, artificial
  primer for PCR amplification of SAG14
aggactacgatgttggtgatgata

SEQ ID NO: 48 - DNA, 24 bp, artificial
  primer for PCR amplification of SAG14
gagtgtgactcaaaagagagcaac

SEQ ID NO: 49 - DNA, 24 bp, artificial
  primer for PCR amplification of SAG15
acgcattgatcataatgacctcta

SEQ ID NO: 50 - DNA, 24 bp, artificial
  primer for PCR amplification of SAG15
atacaatcctccaatggtgaaact

SEQ ID NO: 51 - DNA, 24 bp, artificial
  primer for PCR amplification of SAG101
ggtctcaccactatgttatgcttg

SEQ ID NO: 52 - DNA, 24 bp, artificial
  primer for PCR amplification of SAG101
ttactctcgcaatgacacactttt

SEQ ID NO: 53 - DNA, 24 bp, artificial
  primer for PCR amplification of SIRK
atttatcttgagctgggaagagag

SEQ ID NO: 54 - DNA, 24 bp, artificial
  primer for PCR amplification of SIRK
ggcatacatattattcagcaacca

SEQ ID NO: 55 - DNA, 25 bp, artificial
  primer for PCR amplification of WRKY6
aactgagtccaacaaaattcagaag

SEQ ID NO: 56 - DNA, 24 bp, artificial
  primer for PCR amplification of WRKY6
gtggttcgtaccattgatcataga

SEQ ID NO: 57 - DNA, 24 bp, artificial
  primer for PCR amplification of WRKY53
gaagtgacgtacagaggaacacac

SEQ ID NO: 58 - DNA, 24 bp, artificial
  primer for PCR amplification of WRKY53
tggaagcttacaacaagaagtctg

SEQ ID NO: 59 - DNA, 24 bp, artificial
  primer for PCR amplification of WRKY70
cattttcttggaggaaatatggac

SEQ ID NO: 60 - DNA, 24 bp, artificial
  primer for PCR amplification of WRKY70
gttttccactctacatggcctaat

SEQ ID NO: 61 - DNA, 24 bp, artificial
  primer for PCR amplification of SOD
gatggaagctcctagaggaaatct

SEQ ID NO: 62 - DNA, 24 bp, artificial
  primer for PCR amplification of SOD
ctggaaattacgttctggtttaca

SEQ ID NO: 63 - DNA, 24 bp, artificial
  primer for PCR amplification of CAB
tacaaagagtcagagctcatccac

SEQ ID NO: 64 - DNA, 24 bp, artificial
  primer for PCR amplification of CAB
gtatttgggttcaaaaggttcatc

SEQ ID NO: 65 - DNA, 25 bp, artificial
  primer for PCR amplification of PR-1
atcgtctttgtagctcttgtaggtg

SEQ ID NO: 66 - DNA, 24 bp, artificial
  primer for PCR amplification of PR-1
tgatacatcctgcatatgatgctc

SEQ ID NO: 67 - DNA, 24 bp, artificial
  primer for PCR amplification of PR-2
cttacttcagctacatgggagaca

SEQ ID NO: 68 - DNA, 24 bp, artificial
  primer for PCR amplification of PR-2
caagttcccaatttttaaatacgc

SEQ ID NO: 69 - DNA, 25 bp, artificial
  primer for PCR amplification of PR-5
atggcaaatatctccagtattcaca

SEQ ID NO: 70 - DNA, 24 bp, artificial
  primer for PCR amplification of PR-5
atgtcggggcaagccgcgttgagg

SEQ ID NO: 71 - DNA, 24 bp, artificial
  primer for PCR amplification of EDS1
agatgaatacaagccaaagtgtca

SEQ ID NO: 72 - DNA, 24 bp, artificial
  primer for PCR amplification of EDS1
agcgtaatccaccactttctaaac

SEQ ID NO: 73 - DNA, 24 bp, artificial
  primer for PCR amplification of ACS2
atttcatgggaaaagctagaggtg

SEQ ID NO: 74 - DNA, 24 bp, artificial
  primer for PCR amplification of ACS2
aactttatgccttatccccaacct

SEQ ID NO: 75 - DNA, 25 bp, artificial
  primer for PCR amplification of ACS6
gtaatcgaggagatcgaagattgta

SEQ ID NO: 76 - DNA, 25 bp, artificial
  primer for PCR amplification of ACS6
tactctgccaacacttcttcttctt

SEQ ID NO: 77 - DNA, 24 bp, artificial
  primer for PCR amplification of MPK3
gctataagagatgttgttccacca

SEQ ID NO: 78 - DNA, 25 bp, artificial
  primer for PCR amplification of MPK3
ggcaaagatactaagtagccattcg

SEQ ID NO: 79 - DNA, 24 bp, artificial
  primer for PCR amplification of Calmodulin 38
ccagaagagcttcaaaagagtttc

SEQ ID NO: 80 - DNA, 24 bp, artificial
  primer for PCR amplification of Calmodulin 38
caatcgtatttattgggtcacaaa

SEQ ID NO: 81 - DNA, 24 bp, artificial
  primer for PCR amplification of Choline Kinase1
cgttcttctctgggaaagacttag

SEQ ID NO: 82 - DNA, 24 bp, artificial
  primer for PCR amplification of Choline Kinase1
agaagggaagaaacacacaaactg

SEQ ID NO: 83 - DNA, 24 bp, artificial
  primer for PCR amplification of Jamonate- Responsive 1
acaaggtgactctggtgttgttta

SEQ ID NO: 84 - DNA, 24 bp, artificial
  primer for PCR amplification of Jamonate- Responsive 1
gggatatccagattggtttcacat

SEQ ID NO: 85 - DNA, 24 bp, artificial
  primer for PCR amplification of Wound- Responsive 3
acttctcatatgctcactgatcca

SEQ ID NO: 86 - DNA, 24 bp, artificial
  primer for PCR amplification of Wound- Responsive 3
gaaagaagaagtgtgcaacaagac

SEQ ID NO: 87 - DNA, 24 bp, artificial
  primer for PCR amplification of Actin 2
gacctttaactctcccgctatgta

SEQ ID NO: 88 - DNA, 24 bp, artificial
  primer for PCR amplification of Actin 2
gctttttaagcctttgatcttgag

SEQ ID NO: 89 - DNA, 40 bp, artificial
  primer for PCR amplification of SEQ ID No. 11
acacaccgcgggatatctctttttatattcaaacaataag

SEQ ID NO: 90 - DNA, 38 bp, artificial
  primer for PCR amplification of SEQ ID No. 11
acacactcgagtgttttaggaaagttaaatgacttttg

SEQ ID NO: 91 - DNA, 22 bp, artificial
  primer for PCR amplification of SEQ ID No. 20
aatcagttgtgttcatgaggtg

SEQ ID NO: 92 - DNA, 23 bp, artificial
  primer for PCR amplification of SEQ ID No. 20
agcggataacaatttcacacagg

SEQ ID NO: 93 - DNA, 24 bp, artificial
  primer for PCR amplification of Potato AKIP1/2
ccaaatcagaaaaccctaatttca

SEQ ID NO: 94 - DNA, 24 bp, artificial
  primer for PCR amplification of Potato AKIP1/2
acttggaagtagagccatagcatt

SEQ ID NO: 95 - DNA, 21 bp, artificial
  primer for PCR amplification of Potato AKIP3
tcttctctgagcgaaaatgga

SEQ ID NO: 96 - DNA, 20 bp, artificial
  primer for PCR amplification of Potato AKIP3
tcaggaaatcaccacaacca